(19) United States
(12) Patent Application Publication
(10) Pub. No.: US 2018/0118831 A1
(43) Pub. Date: May 3, 2018

(54) CAR T-CELLS FOR THE TREATMENT OF B7-H4 EXPRESSING SOLID TUMORS

(71) Applicant: University of Southern California, Los Angeles, CA (US)

(72) Inventors: Alan L. Epstein, Pasadena, CA (US); Peisheng Hu, Covina, CA (US)

(73) Assignee: University of Southern California, Los Angeles, CA (US)

(21) Appl. No.: 15/561,968

(22) PCT Filed: Mar. 25, 2016
(86) PCT No.: PCT/US2016/024357

§ 371 Date: Sep. 26, 2017

Related U.S. Application Data
(60) Provisional application No. 62/139,592, filed on Mar. 27, 2015.

(51) Int. Cl. C07K 16/28; A61K 35/17; A61P 35/00; C07K 14/705; C07K 14/725; G01N 33/574

(52) U.S. Cl. CPC C07K 16/2827; A61K 35/17; A61P 35/00; C07K 14/70517; A61K 2039/505; C07K 14/7051; G01N 33/57492; C07K 2319/30; C07K 14/70521

(57) ABSTRACT

CAR cells and antibodies targeting human B7-H4 expressed on many human cancers including but not limited to breast ovarian, and renal cancers are described as a new method of cancer treatment. It is proposed that B7-H4 CAR cells are safe and effective in patients and can be used to treat human tumors expressing the B7-H4 surface protein.

### CROSS-REFERENCE TO RELATED APPLICATIONS

This application claims priority under 35 U.S.C. § 119(e) to U.S. Provisional Application No. 62/139,592, filed Mar. 27, 2015, the content of which is hereby incorporated by reference in its entirety.

### TECHNICAL FIELD

This disclosure relates to novel B7-H4 chimeric antigen receptor (CAR), cells or compositions comprising the same, and methods for using the same for therapy including solid tumors. Also provided herein are isolated peptides and fusion proteins containing immunogenic determinants for the B7-H4 receptor.

### BACKGROUND

The following discussion of the background of the invention is merely provided to aid the reader in the understanding the invention and is not admitted to describe or constitute prior art to the present invention.

Ovarian carcinoma is the most common cause of cancer death from gynecologic tumors and is responsible for approximately 25,000 new cases and 14,000 deaths each year in the United States. Although the overall survival of ovarian carcinoma has improved in the last 30 years to its current rate of 38 months, its 5-year survival for stage III disease has not changed significantly and remains around 25%. Because of the high recurrence rate of these patients, attempts to decrease distant metastases, prolong time to recurrence, and improve overall survival are at the forefront of ovarian cancer research.

In 2014, an estimated 232,670 new cases of invasive breast cancer will be diagnosed in US women and an estimated 40,000 US women will die from metastatic disease. The risk of contracting breast cancer increases with age so that 77% of cases are over the age of 50 at the time of diagnosis. In general, the mortality rate for patients with breast cancer has decreased since 1989 due to earlier detection, improved treatments, and possibly a decreased incidence because of the declining use of postmenopausal hormone therapy. When detected early, the 5-year survival for localized breast cancer is 99%. By contrast, the 5-year survival for regional disease is 84% and importantly, for metastatic disease, it drops precipitously to 24%.

B7-H4 is a B7-like molecule that appears to negatively regulate T cell immunity. Overexpression of B7-H4 reported in 95-100% of breast cancer specimens. Not only is it up-regulated in this tumor type, but its expression is inversely correlated with HER-2 and progesterone receptor status (Tringler, S. et al. (2005) Clin. Cancer Res. 11:1842-1848). Because current therapies employed in breast cancer take advantage of HER-2 (trastuzumab and lapatinib) and progesterone receptor expression (hormone therapy), triple negative breast cancer (negative for estrogen receptor, progesterone receptor, and HER-2) are highly aggressive and are refractory to conventional treatment regimens. B7-H4 is an excellent antigen for targeted therapy, especially since higher over-expression is found in more aggressive and difficult to treat cases.

This year, an estimated 63,920 adults (39,140 men and 24,780 women) in the United States will be diagnosed with renal cancer. It is estimated that 13,860 deaths (8,900 men and 4,960 women) from this disease will occur this year. Renal cancer is the sixth most common cancer and the tenth most common cause of cancer death for men, and it is the eighth most common cause of cancer for women. The five-year survival rate for renal cancer patients is 72%. Approximately 63% of cases do not have metastatic disease at the time of diagnosis. For this group, the five-year survival rate improves to 92%. By contrast, the five-year survival for renal cancer in the pelvis (metastatic disease) is 51%.

### SUMMARY OF THE DISCLOSURE

Provided are novel anti-B7-H4 antibodies and methods of their use diagnostically and therapeutically. In one aspect, In this regard, provide herein is an isolated antibody comprising a heavy chain (HC) immunoglobulin variable domain sequence and a light chain (LC) immunoglobulin variable domain sequence, wherein the antibody binds to an epitope of human B7-H4 comprising the amino acid sequence: IGEDGILSCTFEPDIKLSDIVIQWLKEGVLGLVHEFKEGKDELSEQDEMFRGRTAVFA DQVIVGNASLRLKNVQLTDAGTYKCYIITSKGKGNANLEYKTGAFSMPEVNVDYNA SSETLRCEAPRWFPQPTVVWASQVDQGANFSEVSNTSFELNSENVTMKVVSVLYNV TINNTYSCMIENDIAKATGDIKVTESEIKRRSHLQLLNSKA (SEQ ID NO: 43) or an equivalent thereof.

In certain embodiments disclosed herein, the antibody comprises a heavy chain (HC) immunoglobulin variable domain sequence and a light chain (LC) immunoglobulin variable domain sequence, wherein the antibody binds to an epitope of human B7-H4 comprising, or alternatively consisting essentially of, or yet further consisting of, an amino acid sequence wherein the HC comprises any one of the following a HC CDRH1 comprising the amino acid sequence GFTFSSFG (SEQ ID NO: 2), GFTFSSYG (SEQ ID NO: 3), or GYTFTDY (SEQ ID NO: 4); and/or a HC CDRH2 comprising the amino acid sequence ISSGSSTL (SEQ ID NO: 6), ISSSNSTI (SEQ ID NO: 7), or INPNNGGT (SEQ ID NO: 8); and/or a HC CDRH3 comprising the amino acid sequence ARPLYYYGSVMDY (SEQ ID NO: 10) or RPYYYGSSYDY (SEQ ID NO: 11).

In certain embodiments disclosed herein, the antibody comprises a heavy chain (HC) immunoglobulin variable domain sequence and a light chain (LC) immunoglobulin variable domain sequence, wherein the antibody binds to an epitope of human B7-H4 comprising, or alternatively consisting essentially of, or yet further consisting of, an amino acid sequence wherein the LC comprises a LC CDRL1 comprising the amino acid QSIVHRNGNTY (SEQ ID NO: 19), QSIVHSNGNTY (SEQ ID NO: 20), or ENIGSY (SEQ ID NO: 21); and/or a LC CDRL2 comprising the amino acid sequence KVS (SEQ ID NO: 22) or AAT (SEQ ID NO: 23); and/or a LC CDRL3 comprising the amino acid sequence FQGSYVPPT (SEQ ID NO: 25), FQGSHVPLT (SEQ ID NO: 26), QHYYSTLVT (SEQ ID NO: 27).

Some aspects of the disclosure relate to a chimeric antigen receptor (CAR) comprising an antigen binding domain specific to B7-H4—for example, the antigen binding domain of an anti-B7-H4 antibody, nucleic acids encoding them as well as method for the production and use of them.

Aspects of the disclosure relate to a chimeric antigen receptor (CAR) comprising: (a) an antigen binding domain of an B7-H4 antibody; (b) a hinge domain; (c) a transmembrane domain; and (d) an intracellular domain. Further aspects of the disclosure relate to a chimeric antigen receptor (CAR) comprising: (a) an antigen binding domain of a B7-H4 antibody; (b) a hinge domain; (c) a CD28 transmembrane domain; (d) one or more costimulatory regions selected from a CD28 costimulatory signaling region, a 4-1BB costimulatory signaling region, an ICOS costimulatory signaling region, and an OX40 costimulatory region; and (e) a CD3 zeta signaling domain or an equivalent or alternative thereof.

In a further aspect, the present disclosure provides a chimeric antigen receptor (CAR) comprising: (a) an antigen binding domain of an anti-B7-H4 antibody, (b) a CD8 α hinge domain; (c) a CD8 α transmembrane domain; (d) a 4-1BB costimulatory signaling region; and (e) a CD3 zeta signaling domain or an equivalent or alternative thereof.

Further aspects of the disclosure relate to an isolated nucleic acid sequence encoding the antibodies, vectors, and host cells containing them.

Other aspects of the disclosure relate to an isolated cell comprising a B7-H4 CAR and methods of producing such cells. Still other method aspects of the disclosure relate to methods for inhibiting the growth of a tumor, e.g., a solid tumor, and treating a cancer patient comprising administering an effective amount of the isolated cell.

Further method aspects of the disclosure relate to methods and kits for determining if a patient is likely to respond or is not likely to B7-H4 CAR therapy through use of either or both the B7-H4 antibody and the B7-H4 CAR cells.

Additional aspects of the disclosure relate to compositions comprising a carrier and one or more of the products described in the embodiments disclosed herein. In some aspects, the present disclosure provides a composition comprising a carrier and one or more of: the B7-H4 antibody; and/or the B7-H4 CAR; and/or the isolated nucleic acid encoding the B7-H4 antibody or the B7-H4 CAR; and/or the vector comprising the isolated nucleic acid sequence encoding the B7-H4 antibody, or the B7-H4 CAR; and/or an isolated cell comprising the B7-H4 CAR.

### BRIEF DESCRIPTION OF THE DRAWINGS

FIGS. 1A-1C show a schematic diagram and HPLC Analysis of Human B7-H4-Fc Fusion Protein Used as Antigen. (FIG. 1A) The vector used to construct the gene; (FIG. 1B) the completed B7-H4-Fc fusion protein in which the B7-H4 was fused to the N-terminus of the immunoglobulin Fc region of human IgG1 producing a dimeric protein used as antigen. (FIG. 1C) HPLC analysis of purified B7-H4-Fc showing the expected retention time indicative of its molecular weight.

FIG. 2 shows representative flow cytometry data for mouse monoclonal anti-human B7-H4 on SKBR-3, HT-29, JAR, and T47D cell lines derived from breast adenocarcinoma, colorectal adenocarcinoma, choriocarcinoma, and breast ductal carcinoma, respectively. Darker line represents cells stained for B7-H4, and lighter line represents cells stained with isotype control. A sheep anti-mouse IgG conjugated to FITC was used as the secondary antibody. Cell surface expression of B7-H4 matches q-PCR data for b7-h4 expression in these cell lines (data not shown).

FIG. 3 shows flow cytometry screening data of newly generated and purified monoclonal antibodies to human B7-H4. Subclones of positive hybridomas (35-8 and 5F6-6) were selected for the generation of CAR T-cells based upon these results. Clone 35-8 was then sequenced and used to produce B7-H4 CAR T-cells for immunotherapy.

FIGS. 4A-4B show representative images of B7-H4 antibody (clone #35-8) staining on normal and cancer tissue microarrays. (FIG. 4A) B7-H4 staining on normal tissues. (FIG. 4B) B7-H4 staining on normal and cancer tissue of the breast. Other normal tissues found negative for B7-H4 positivity (not shown) include the following: adrenal gland, bone marrow, cerebellum, esophagus, hypophysis, intestine, lymph node, ovary, prostate, stomach, testis, thyroid, thymus, tongue, uterine, skin, and nerve tissue.

FIG. 5 shows a schematic diagram of the DNA sequence for, and the theoretical structure of third generation anti-B7-H4 CAR in the plasma membrane.

FIG. 6 shows immunohistochemistry staining of B7-H4 on sections of (A) human breast carcinoma biopsy and (B) SKBR3 human breast cancer cell line pellet showing cell surface positivity for antigen (brown staining).

FIG. 7 shows a schematic representation of the gene transfer vector and of the transgene. The backbone of the gene transfer vector is an HIV-based, bicistronic lentiviral vector, pLVX-IRES-ZsGreen containing HIV-1 5′ and 3′ long terminal repeats (LTRs), packaging signal (Ψ), EF1α promoter, internal ribosome entry site (IRES), ZsGreen, a green fluorescent protein, woodchuck hepatitis virus post-transcriptional regulatory element (WPRE), and simian virus 40 origin (SV40). Constitutive expression of the transgene comprising of a scFV specific to B7-H4, a CD8 hinge and transmembrane region and CD28, 4-1BB and CD3ζ signaling domain, is insured by the presence of the EF-1α promoter. Expression of the detection protein, ZsGreen is carried out by the IRES region. Integration of the vector can be assayed by the presence of ZsGreen in the cells, via fluorescent microscopy

FIG. 8 shows cytotoxicity of the B7-H4 CAR T-cells. Cytotoxicity of the B7-H4 CAR expressing T-cells was determined using an LDH cytotoxicity kit as described in the Methods. Prior to the assay, T-cells were activated using αCD3/CD8 beads (Stem Cell Technologies, 30 μl to 2 ml of media). The activated T-cells were transduced with B7-H4 lentiviral particles, following which the T cells were activated for using the αCD3/CD8 beads. Un-transduced, activated T-cells were used as a control. 3000 SKBR3 cells were plated per well. B7-H4 transduced T cells were added in ratios of 20:1, 10:1, 5:1 and 1:1 (60,000-3000 cells) to the wells. Each data point represents the average of triplicate measurements.

### DETAILED DESCRIPTION

It is to be understood that the present disclosure is not limited to particular aspects described, as such may, of course, vary. It is also to be understood that the terminology used herein is for the purpose of describing particular aspects only, and is not intended to be limiting, since the scope of the present disclosure will be limited only by the appended claims.

Unless defined otherwise, all technical and scientific terms used herein have the same meanings as commonly understood by one of ordinary skill in the art to which this technology belongs. Although any methods and materials similar or equivalent to those described herein can be used in the practice or testing of the present technology, the preferred methods, devices and materials are now described. All technical and patent publications cited herein are incorporated herein by reference in their entirety. Nothing herein is to be construed as an admission that the present technology is not entitled to antedate such disclosure by virtue of prior invention.

The practice of the present technology will employ, unless otherwise indicated, conventional techniques of tissue culture, immunology, molecular biology, microbiology, cell biology, and recombinant DNA, which are within the skill of the art. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligomicleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning, Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

It is to be inferred without explicit recitation and unless otherwise intended, that when the present technology relates to a polypeptide, protein, polynucleotide or antibody, an equivalent or a biologically equivalent of such is intended within the scope of the present technology.

### Definitions

As used in the specification and claims, the singular form “a”, “an”, and “the” include plural references unless the context clearly dictates otherwise. For example, the term “a cell” includes a plurality of cells, including mixtures thereof.

As used herein, the term “animal” refers to living multi-cellular vertebrate organisms, a category that includes, for example, mammals and birds. The term “mammal” includes both human and non-human mammals.

The terms “subject,” “host,” “individual,” and “patient” are as used interchangeably herein to refer to human and veterinary subjects, for example, humans, animals, non-human primates, dogs, cats, sheep, mice, horses, and cows. In some embodiments, the subject is a human.

As used herein, the term “antibody” collectively refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits and mice, as well as non-mammalian species, such as shark immunoglobulins. Unless specifically noted otherwise, the term “antibody” includes intact immunoglobulins and “antibody fragments” or “antigen binding fragments” that specifically bind to a molecule of interest (or a group of highly similar molecules of interest) to the substantial exclusion of binding to other molecules (for example, antibodies and antibody fragments that have a binding constant for the molecule of interest that is at least 10³ M⁻¹ greater, at least 10⁴ M⁻¹ greater or at least 10⁵ M⁻¹ greater than a binding constant for other molecules in a biological sample). The term “antibody” also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.), Kuby, J., Immunology, 3^{rd} Ed., W.H. Freeman & Co., New York, 1997.

In terms of antibody structure, an immunoglobulin has heavy (H) chains and light (L) chains interconnected by disulfide bonds. There are two types of light chain, lambda (k) and kappa (κ). There are five main heavy chain classes (or isotypes) which determine the functional activity of an antibody molecule: IgM, IgD, IgG, IgA and IgE. Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species. The framework region of an antibody, that is the combined framework regions of the constituent light and heavy chains, largely adopts a β-sheet conformation and the CDRs form loops which connect, and in some cases form part of, the β-sheet structure. Thus, framework regions act to form a scaffold that provides for positioning the CDRs in correct orientation by inter-chain, non-covalent interactions.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. An antibody that binds B7-H4 will have a specific V_{H} region and the V_{L} region sequence, and thus specific CDR sequences. Antibodies with different specificities (i.e. different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

As used herein, the term “antigen” refers to a compound, composition, or substance that may be specifically bound by the products of specific humoral or cellular immunity, such as an antibody molecule or T-cell receptor. Antigens can be any type of molecule including, for example, haptens, simple intermediary metabolites, sugars (e.g., oligosaccharides), lipids, and hormones as well as macromolecules such as complex carbohydrates (e.g., polysaccharides), phospholipids, and proteins. Common categories of antigens include, but are not limited to, viral antigens, bacterial antigens, fungal antigens, protozoa and other parasitic antigens, tumor antigens, antigens involved in autoimmune disease, allergy and graft rejection, toxins, and other miscellaneous antigens.

As used herein, the term “antigen binding domain” or “antigen binding fragment” refers to any protein or polypeptide domain that can specifically bind to an antigen target.

The term “chimeric antigen receptor” (CAR), as used herein, refers to a fused protein comprising an extracellular domain capable of binding to an antigen, a transmembrane domain derived from a polypeptide different from a polypeptide from which the extracellular domain is derived, and at least one intracellular domain. The “chimeric antigen receptor (CAR)” is sometimes called a “chimeric receptor”, a “T-body”, or a “chimeric immune receptor (CIR).” The “extracellular domain capable of binding to an antigen” means any oligopeptide or polypeptide that can bind to a certain antigen. The “intracellular domain” means any oligopeptide or polypeptide known to function as a domain that transmits a signal to cause activation or inhibition of a biological process in a cell. The “transmembrane domain” means any oligopeptide or polypeptide known to span the cell membrane and that can function to link the extracellular and signaling domains. A chimeric antigen receptor may optionally comprise a “hinge domain” which serves as a linker between the extracellular and transmembrane domains. Non-limiting exemplary polynucleotide sequences that encode for components of each domain are disclosed herein, e.g.:

Hinge domain: IgG1 heavy chain hinge sequence,
SEQ. ID NO: 53:
CTCGAGCCCAAATCTTGTGACAAAACTCACACATGCCCACCGTGCCCG
Transmembrane domain: CD28 transmembran region
SEQ. ID NO: 54:
TTTTGGGTGCTGGTGGTGGTTGGTGGAGTCCTGGCTTGCTATAGCTTGCT
AGTAACAGIGGCCTTTATTATTTTCTGGGTG
Intracellular domain: 4-IBB co-stimulatory
signaling region, SEQ. ID NO: 55:
AAACGGGGCAGAAAGAAACTCCTGTATATATTCAAACAACCATTTATGAG
ACCAGTACAAACTACTCAAGAGGAAGATGGCTGTAGCTGCCGATTTCCAG
AAGAAGAAGAAGGAGGATGTGAACTG
Intracellular domain: CD28 co-stimulatory
signaling region, SEQ. ID NO: 56:
AGGAGTAAGAGGAGCAGGCTCCTGCACAGTGACTACATGAACATGACTCC
CCGCCGCCCCGGGCCCACCCGCAAGCATTACCAGCCCTATGCCCCACCAC
GCGACTTCGCAGCCTATCGCTCC
Intracellular domain: CD3 zeta signaling region,
SEQ. ID NO: 57:
AGAGTGAAGTTCAGCAGGAGCGCAGACGCCCCCGCGTACCAGCAGGGCCA
GAACCAGCTCTATAACGAGCTCAATCTAGGACGAAGAGAGGAGTACGATG
TTTTGGACAAGAGACGTGGCCGGGACCCTGAGATGGGGGGAAAGCCGAGA
AGGAAGAACCCTCAGGAAGGCCTGTACAATGAACTGCAGAAAGATAAGAT
GGCGGAGGCCTACAGTGAGATTGGGATGAAAGGCGAGCGCCGGAGGGGCA
AGGGGCACGATGGCCTTACCAGGGTCTCAGTACAGCCACCAAGGACACCT
ACGACGCCCTTCACATGCAGGCCCTGCCCCCTCGCTAA

Further embodiments of each exemplary domain component include other proteins that have analogous biological function that share at least 70%, or alternatively at least 80% amino acid sequence identity, preferably 90% sequence identity, more preferably at least 95% sequence identity with the proteins encoded by the above disclosed nucleic acid sequences. Further, non limiting examples of such domains are provided herein.

A “composition” typically intends a combination of the active agent, e.g., compound or composition, and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri-, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

The term “consensus sequence” as used herein refers to an amino acid or nucleic acid sequence that is determined by aligning a series of multiple sequences and that defines an idealized sequence that represents the predominant choice of amino acid or base at each corresponding position of the multiple sequences. Depending on the sequences of the series of multiple sequences, the consensus sequence for the series can differ from each of the sequences by zero, one, a few, or more substitutions. Also, depending on the sequences of the series of multiple sequences, more than one consensus sequence may be determined for the series. The generation of consensus sequences has been subjected to intensive mathematical analysis. Various software programs can be used to determine a consensus sequence.

As used herein, the term “B7-H4” (also known as VTCN1, H4, B7h.5, B7S1, B7X, or PRO 129) refers to a specific molecule associated with this name and any other molecules that have analogous biological function that share at least 80% amino acid sequence identity, preferably 90% sequence identity, more preferably at least 95% sequence identity with B7-H4. Examples of the B7-H4 sequence are provided herein. In addition, the protein sequences associated with GenBank Accession Nos. AY280973.1 (Mus musculus) and NP_078902 (Homo sapiens) provide example sequences of B7-H4 in various animals; the referenced genes have 87% homology. The sequences associated with each of the listed GenBank Accession Nos. are herein incorporated by reference. As used herein, the term “anti-B7-H4,” in reference to an antibody or receptor, refers to an antibody or receptor that specifically binds to B7-H4 and includes reference to any antibody which is generated against B7-H4.

As used herein, the term “CD8 α hinge domain” refers to a specific protein fragment associated with this name and any other molecules that have analogous biological function that share at least 70%, or alternatively at least 80% amino acid sequence identity, preferably 90% sequence identity, more preferably at least 95% sequence identity with the CD8 α hinge domain sequence as shown herein. The example sequences of CD8 α hinge domain for human, mouse, and other species are provided in Pinto, R. D. et al. (2006) Vet. Immunol. Immunopathol. 110:169-177. The sequences associated with the CD8 α hinge domain are provided in Pinto, R. D. et al. (2006) Vet. Immunol. Immunopathol. 110:169-17. Non-limiting examples of such include:

Human CD8 alpha hinge domain, SEQ. ID NO: 45:
PAKPTTTPAPRPPTPAPTIASQPLSLRPEACRPAAGGAVHTRGLDFACDIY
Mouse CD8 alpha hinge domain, SEQ. ID NO: 46:
KVNSTTTKPVLRTPSPVHPTGTSQPQRPEDCRPRGSVKGTGLDFACDIY
Cat CD8 alpha hinge domain, SEQ. ID NO: 47:
PVKPTTTPAPRPPTQAPITTSQRVSLRPGTCQPSAGSTVEASGLDLSCDIY

As used herein, the term “CD8 α transmembrane domain” refers to a specific protein fragment associated with this name and any other molecules that have analogous biological function that share at least 70%, or alternatively at least 80% amino acid sequence identity, preferably 90% sequence identity, more preferably at least 95% sequence identity with the CD8 α transmembrane domain sequence as shown herein. The fragment sequences associated with the amino acid positions 183 to 203 of the human T-cell surface glycoprotein CD8 alpha chain (NCBI Reference Sequence: NP_001759.3), or the amino acid positions 197 to 217 of the mouse T-cell surface glycoprotein CD8 alpha chain (NCBI Reference Sequence: NP_001074579.1), and the amino acid positions 190 to 210 of the rat T-cell surface glycoprotein CD8 alpha chain (NCBI Reference Sequence: NP_113726.1) provide additional example sequences of the CD8 α transmembrane domain. The sequences associated with each of the listed NCBI are provided as follows:

Human CD8 alpha transmembrane domain,
SEQ. ID NO: 48:
IYIWAPLAGTCGVLLLSLVIT
Mouse CD8 alpha transmembrane domain,
SEQ. ID NO: 49:
IWAPLAGICVALLLSLIITLI
Rat CD8 alpha transmembrane domain,
SEQ. ID NO: 50:
IWAPLAGICAVLLLSLVITLI

As used herein, the term “CD28 transmembrane domain” refers to a specific protein fragment associated with this name and any other molecules that have analogous biological function that share at least 70%, or alternatively at least 80% amino acid sequence identity, at least 90% sequence identity, or alternatively at least 95% sequence identity with the CD28 transmembrane domain sequence as shown herein. The fragment sequences associated with the GenBank Accession Nos: XM_006712862.2 and XM_009444056.1 provide additional, non-limiting, example sequences of the CD28 transmembrane domain. The sequences associated with each of the listed accession numbers are provided as follows the sequence encoded by SEQ ID NO: 56.

As used herein, the term “4-1BB costimulatory signaling region” refers to a specific protein fragment associated with this name and any other molecules that have analogous biological function that share at least 70%, or alternatively at least 80% amino acid sequence identity, preferably 90% sequence identity, more preferably at least 95% sequence identity with the 4-1BB costimulatory signaling region sequence as shown herein. The example sequences of the 4-1 BB costimulatory signaling region are provided in U.S. Publication No. US20130266551A1 (filed as U.S. App. No. U.S. Ser. No. 13/826,258). The sequence of the 4-1BB costimulatory signaling region associated disclosed in U.S. Publication No. US20130266551A1 is listed as follows:

The 4-1BB costimulatory signaling region,
SEQ. ID NO: 51:
KRGRKKLLYIFKQPFMRPVQTTQEEDGCSCRFPEEEEGGCEL

As used herein, the term “CD28 costimulatory signaling region” refers to a specific protein fragment associated with this name and any other molecules that have analogous biological function that share at least 700, or alternatively at least 80% amino acid sequence identity, preferably 90% sequence identity, more preferably at least 95% sequence identity with the CD28 costimulatory signaling region sequence shown herein. Exemplary CD28 costimulatory signaling domains are provided in U.S. Pat. No. 5,686,281; Geiger, T. L. et al., Blood 98: 2364-2371 (2001); Hombach, A. et al., J Immunol 167: 6123-6131 (2001); Maher, J. et al. Nat Biotechnol 20: 70-75 (2002); Haynes, N. M. et al., J Immunol 169: 5780-5786 (2002); Haynes, N. M. et al., Blood 100: 3155-3163 (2002). Non-limiting examples include residues 114-220 of the below CD28 Sequence: MLRLLLALNL FPSIQVTGNK ILVKQSPMLV AYDNAVNLSC KYSYNLFSRE FRASLHKGLDSAVEVCVVYG NYSQQLQVYS KTGFNCDGKL GNESVTFYLQ NLYVNQTDIY FCKIEVMYPPPYLDNEKSNG TIIHVKGKHL CPSPLFPGPS KPFWVLVVVG GVLACYSLLVTVAFIIFWVR SKRSRLLHSD YMNMTPRRPG PTRKHYQPYA PPRDFAAYRS (SEQ ID NO: 58), and equivalents thereof.

As used herein, the term “ICOS costimulatory signaling region” refers to a specific protein fragment associated with this name and any other molecules that have analogous biological function that share at least 70%, or alternatively at least 80% amino acid sequence identity, preferably 90% sequence identity, more preferably at least 95% sequence identity with the ICOS costimulatory signaling region sequence as shown herein. Non-limiting example sequences of the ICOS costimulatory signaling region are provided in U.S. Publication 2015/0017141A1 the exemplary polynucleotide sequence provided below.

ICOS costimulatory signaling region,
SEQ ID NO: 59:
ACAAAAAAGA AGTATTCATC CAGTGTGCAC GACCCTAACG
GTGAATACAT GTTCATGAGA GCAGTGAACA CAGCCAAAAA
ATCCAGACTC ACAGATGTGA CCCTA

As used herein, the term “OX40 costimulatory signaling region” refers to a specific protein fragment associated with this name and any other molecules that have analogous biological function that share at least 70%, or alternatively at least 80% amino acid sequence identity, or alternatively 90% sequence identity, or alternatively at least 95% sequence identity with the OX40 costimulatory signaling region sequence as shown herein. Non-limiting example sequences of the OX40 costimulatory signaling region are disclosed in U.S. Publication 2012/20148552A1, and include the exemplary sequence provided below.

OX40 costimulatory signaling region,
SEQ ID NO: 60:
AGGGACCAG AGGCTGCCCC CCGATGCCCA CAAGCCCCCT
GGGGGAGGCA GTTTCCGGAC CCCCATCCAA GAGGAGCAGG
CCGACGCCCA CTCCACCCTG GCCAAGATC

As used herein, the term “CD3 zeta signaling domain” refers to a specific protein fragment associated with this name and any other molecules that have analogous biological function that share at least 70%, or alternatively at least 80% amino acid sequence identity, preferably 90% sequence identity, more preferably at least 95% sequence identity with the CD3 zeta signaling domain sequence as shown herein. The example sequences of the CD3 zeta signaling domain are provided in U.S. Publication No. US20130266551A1. The sequence associated with the CD3 zeta signaling domain is listed as follows:

The CD3 zeta signaling domain, SEQ. ID NO: 52:
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR

As used herein, the term “B cell,” refers to a type of lymphocyte in the humoral immunity of the adaptive immune system. B cells principally function to make antibodies, serve as antigen presenting cells, release cytokines, and develop memory B cells after activation by antigen interaction. B cells are distinguished from other lymphocytes, such as T cells, by the presence of a B-cell receptor on the cell surface. B cells may either be isolated or obtained from a commercially available source. Non-limiting examples of commercially available B cell lines include lines AHH-1 (ATCC® CRL-8146™), BC-1 (ATCC® CRL-2230™), BC-2 (ATCC® CRL-2231™), BC-3 (ATCC® CRL-2277™), CA46 (ATCC® CRL-1648™), DG-75 [D.G.-75] (ATCC® CRL-2625™), DS-1 (ATCC® CRL-11102™), EB-3 [EB3] (ATCC® CCL-85™), Z-138 (ATCC #CRL-3001), DB (ATCC CRL-2289), Toledo (ATCC CRL-2631), Pfiffer (ATCC CRL-2632), SR (ATCC CRL-2262), JM-1 (ATCC CRL-10421), NFS-5 C-1 (ATCC CRL-1693); NFS-70 C10 (ATCC CRL-1694), NFS-25 C-3 (ATCC CRL-1695), AND SUP-B15 (ATCC CRL-1929). Further examples include but are not limited to cell lines derived from anaplastic and large cell lymphomas, e.g., DEL, DL-40, FE-PD, JB6, Karpas 299, Ki-JK, Mac-2A Ply1, SR-786, SU-DHL-1, -2, -4, -5, -6, -7, -8, -9, -10, and -16, DOHH-2, NU-DHL-1, U-937, Granda 519, USC-DHL-1, RL; Hodgkin's lymphomas, e.g., DEV, HD-70, HDLM-2, HD-MyZ, HKB-1, KM-H2, L 428, L 540, L1236, SBH-1, SUP-HD1, SU/RH-HD-1. Non-limiting exemplary sources for such commercially available cell lines include the American Type Culture Collection, or ATCC, (www.atcc.org/) and the German Collection of Microorganisms and Cell Cultures (https://www.dsmz.de/).

As used herein, the term “T cell,” refers to a type of lymphocyte that matures in the thymus. T cells play an important role in cell-mediated immunity and are distinguished from other lymphocytes, such as B cells, by the presence of a T-cell receptor on the cell surface. T-cells may either be isolated or obtained from a commercially available source. “T cell” includes all types of immune cells expressing CD3 including T-helper cells (CD4+ cells), cytotoxic T-cells (CD8+ cells), natural killer T-cells, T-regulatory cells (Treg) and gamma-delta T cells. A “cytotoxic cell” includes CD8+ T cells, natural-killer (NK) cells, and neutrophils, which cells are capable of mediating cytotoxicity responses. Non-limiting examples of commercially available T-cell lines include lines BCL2 (AAA) Jurkat (ATCC® CRL-2902™), BCL2 (S70A) Jurkat (ATCC® CRL-2900™), BCL2 (S87A) Jurkat (ATCC® CRL-2901™), BCL2 Jurkat (ATCC® CRL-2899™), Neo Jurkat (ATCC® CRL-2898™), TALL-104 cytotoxic human T cell line (ATCC # CRL-11386). Further examples include but are not limited to mature T-cell lines, e.g., such as Deglis, EBT-8, HPB-MLp-W, HUT 78, HUT 102, Karpas 384, Ki 225, My-La, Se-Ax, SKW-3, SMZ-1 and T34; and immature T-cell lines, e.g., ALL-SIL, Bel3, CCRF-CEM, CML-T1, DND-41, DU.528, EU-9, HD-Mar, HPB-ALL, H-SB2, HT-1, JK-T1, Jurkat, Karpas 45, KE-37, KOPT-K1, K-T1, L-KAW, Loucy, MAT, MOLT-1, MOLT 3, MOLT-4, MOLT 13, MOLT-16, MT-1, MT-ALL, P12/Ichikawa, Peer, PER0117, PER-255, PF-382, PFI-285, RPMI-8402, ST-4, SUP-T1 to T14, TALL-1, TALL-101, TALL-103/2, TALL-104, TALL-105, TALL-106, TALL-107, TALL-197, TK-6, TLBR-1, -2, -3, and -4, CCRF-HSB-2 (CCL-120.1), J.RT3-T3.5 (ATCC TIB-153), J45.01 (ATCC CRL-1990), J.CaM1.6 (ATCC CRL-2063), RS4;11 (ATCC CRL-1873), CCRF-CEM (ATCC CRM-CCL-119); and cutaneous T-cell lymphoma lines, e.g., HuT78 (ATCC CRM-TIB-161), MJ[G11] (ATCC CRL-8294), HuT102 (ATCC TIB-162). Null leukemia cell lines, including but not limited to REH, NALL-1, KM-3, L92-221, are a another commercially available source of immune cells, as are cell lines derived from other leukemias and lymphomas, such as K562 erythroleukemia, THP-1 monocytic leukemia, U937 lymphoma, HEL erythroleukemia, HL60 leukemia, HMC-1 leukemia, KG-1 leukemia, U266 myeloma. Non-limiting exemplary sources for such commercially available cell lines include the American Type Culture Collection, or ATCC, (http://www.atcc.org/) and the German Collection of Microorganisms and Cell Cultures (https://www.dsmz.de/).

As used herein, the term “NK cell,” also known as natural killer cell, refers to a type of lymphocyte that originates in the bone marrow and play a critical role in the innate immune system. NK cells provide rapid immune responses against viral-infected cells, tumor cells or other stressed cell, even in the absence of antibodies and major histocompatibility complex on the cell surfaces. NK cells may either be isolated or obtained from a commercially available source. Non-limiting examples of commercial NK cell lines include lines NK-92 (ATCC® CRL-2407™), NK-92MI (ATCC® CRL-2408™). Further examples include but are not limited to NK lines HANK1, KHYG-1, NKL, NK-YS, NOI-90, and YT. Non-limiting exemplary sources for such commercially available cell lines include the American Type Culture Collection, or ATCC, (http://www.atcc.org/) and the German Collection of Microorganisms and Cell Cultures (https://www.dsmz.de/).

As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

As used herein, the term “vector” refers to a nucleic acid construct deigned for transfer between different hosts, including but not limited to a plasmid, a virus, a cosmid, a phage, a BAC, a YAC, etc. In some embodiments, plasmid vectors may be prepared from commercially available vectors. In other embodiments, viral vectors may be produced from baculoviruses, retroviruses, adenoviruses, AAVs, etc. according to techniques known in the art. In one embodiment, the viral vector is a lentiviral vector.

The term “promoter” as used herein refers to any sequence that regulates the expression of a coding sequence, such as a gene. Promoters may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors.

As used herein, the term “isolated cell” generally refers to a cell that is substantially separated from other cells of a tissue. “Immune cells” includes, e.g., white blood cells (leukocytes) which are derived from hematopoietic stem cells (HSC) produced in the bone marrow, lymphocytes (T cells, B cells, natural killer (NK) cells) and myeloid-derived cells (neutrophil, eosinophil, basophil, monocyte, macrophage, dendritic cells). “T cell” includes all types of immune cells expressing CD3 including T-helper cells (CD4+ cells), cytotoxic T-cells (CD8+ cells), natural killer T-cells, T-regulatory cells (Treg) and gamma-delta T cells. A “cytotoxic cell” includes CD8+ T cells, natural-killer (NK) cells, and neutrophils, which cells are capable of mediating cytotoxicity responses.

The term “transduce” or “transduction” as it is applied to the production of chimeric antigen receptor cells refers to the process whereby a foreign nucleotide sequence is introduced into a cell. In some embodiments, this transduction is done via a vector.

As used herein, the term “autologous,” in reference to cells refers to cells that are isolated and infused back into the same subject (recipient or host). “Allogeneic” refers to non-autologous cells.

An “effective amount” or “efficacious amount” refers to the amount of an agent, or combined amounts of two or more agents, that, when administered for the treatment of a mammal or other subject, is sufficient to effect such treatment for the disease. The “effective amount” will vary depending on the agent(s), the disease and its severity and the age, weight, etc., of the subject to be treated.

A “solid tumor” is an abnormal mass of tissue that usually does not contain cysts or liquid areas. Solid tumors can be benign or malignant. Different types of solid tumors are named for the type of cells that form them. Examples of solid tumors include sarcomas, carcinomas, and lymphomas.

The term “ovarian cancer” refers to a type of cancer that forms in issues of the ovary, and has undergone a malignant transformation that makes the cells within the cancer pathological to the host organism with the ability to invade or spread to other parts of the body. The ovarian cancer herein comprises type I cancers of low histological grade and type II cancer of higher histological grade. Particularly, the ovarian cancer includes but is not limited to epithelial carcinoma, serous carcinoma, clear-cell carcinoma, sex cord stromal tumor, germ cell tumor, dysgerminoma, mixed tumors, secondary ovarian cancer, low malignant potential tumors.

The term “prostate cancer” refers to a type of cancer that develops in the prostate, a gland in the male reproductive system. The prostate cancer herein includes but is not limited to adenocarcinoma, sarcomas, small cell carcinomas, neuroendocrine tumors, transitional cell carcinomas.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. For example, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions disclosed herein. Aspects defined by each of these transition terms are within the scope of the present disclosure.

As used herein, the term “detectable marker” refers to at least one marker capable of directly or indirectly, producing a detectable signal. A non-exhaustive list of this marker includes enzymes which produce a detectable signal, for example by colorimetry, fluorescence, luminescence, such as horseradish peroxidase, alkaline phosphatase, β-galactosidase, glucose-6-phosphate dehydrogenase, chromophores such as fluorescent, luminescent dyes, groups with electron density detected by electron microscopy or by their electrical property such as conductivity, amperometry, voltammetry, impedance, detectable groups, for example whose molecules are of sufficient size to induce detectable modifications in their physical and/or chemical properties, such detection may be accomplished by optical methods such as diffraction, surface plasmon resonance, surface variation, the contact angle change or physical methods such as atomic force spectroscopy, tunnel effect, or radioactive molecules such as ³² P, ³⁵ S or ¹²⁵ I.

As used herein, the term “purification marker” refers to at least one marker useful for purification or identification. A non-exhaustive list of this marker includes His, lacZ, GST, maltose-binding protein, NusA, BCCP, c-myc, CaM, FLAG, GFP, YFP, cherry, thioredoxin, poly(NANP), V5, Snap, HA, chitin-binding protein, Soflag 1, Softag 3, Strep, or S-protein. Suitable direct or indirect fluorescence marker comprise FLAG, GFP, YFP, RFP, dTomato, cherry, Cy3, Cy 5, Cy 5.5, Cy 7, DNP, AMCA, Biotin, Digoxigenin, Tamra, Texas Red, rhodamine, Alexa fluors, FITC, TRITC or any other fluorescent dye or hapten.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample. In one aspect, the expression level of a gene from one sample may be directly compared to the expression level of that gene from a control or reference sample. In another aspect, the expression level of a gene from one sample may be directly compared to the expression level of that gene from the same sample following administration of a compound.

As used herein, “homology” or “identical”, percent “identity” or “similarity”, when used in the context of two or more nucleic acids or polypeptide sequences, refers to two or more sequences or subsequences that are the same or have a specified percentage of nucleotides or amino acid residues that are the same, e.g., at least 60% identity, preferably at least 65%, 70%, 75%, 80%, 85%, 90%, 91%, 92%, 93%, 94%, 95%, 96%, 97%, 98%, 99%, or higher identity over a specified region (e.g., nucleotide sequence encoding an antibody described herein or amino acid sequence of an antibody described herein). Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. The terms “homology” or “identical”, percent “identity” or “similarity” also refer to, or can be applied to, the complement of a test sequence. The terms also include sequences that have deletions and/or additions, as well as those that have substitutions. As described herein, the preferred algorithms can account for gaps and the like. Preferably, identity exists over a region that is at least about 25 amino acids or nucleotides in length, or more preferably over a region that is at least 50-100 amino acids or nucleotides in length. An “unrelated” or “non-homologous” sequence shares less than 40% identity, or alternatively less than 25% identity, with one of the sequences disclosed herein.

The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as “primary therapy and primary treatment.” See National Cancer Institute website at www.cancer.gov, last visited on May 1, 2008. Typically, a patient is given a subsequent chemotherapy regimen because the patient did not show a positive clinical or sub-clinical response to the first line therapy or the first line therapy has stopped.

In one aspect, the term “equivalent” or “biological equivalent” of an antibody means the ability of the antibody to selectively bind its epitope protein or fragment thereof as measured by ELISA or other suitable methods. Biologically equivalent antibodies include, but are not limited to, those antibodies, peptides, antibody fragments, antibody variant, antibody derivative and antibody mimetics that bind to the same epitope as the reference antibody.

It is to be inferred without explicit recitation and unless otherwise intended, that when the present disclosure relates to a polypeptide, protein, polynucleotide or antibody, an equivalent or a biologically equivalent of such is intended within the scope of this disclosure. As used herein, the term “biological equivalent thereof” is intended to be synonymous with “equivalent thereof” when referring to a reference protein, antibody, polypeptide or nucleic acid, intends those having minimal homology while still maintaining desired structure or functionality. Unless specifically recited herein, it is contemplated that any polynucleotide, polypeptide or protein mentioned herein also includes equivalents thereof. For example, an equivalent intends at least about 70% homology or identity, or at least 80% homology or identity and alternatively, or at least about 85%, or alternatively at least about 90%, or alternatively at least about 95%, or alternatively 98% percent homology or identity and exhibits substantially equivalent biological activity to the reference protein, polypeptide or nucleic acid. Alternatively, when referring to polynucleotides, an equivalent thereof is a polynucleotide that hybridizes under stringent conditions to the reference polynucleotide or its complement.

A polynucleotide or polynucleotide region (or a polypeptide or polypeptide region) having a certain percentage (for example, 800, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

A “normal cell corresponding to the tumor tissue type” refers to a normal cell from a same tissue type as the tumor tissue. A non-limiting example is a normal lung cell from a patient having lung tumor, or a normal colon cell from a patient having colon tumor.

The term “isolated” as used herein refers to molecules or biologicals or cellular materials being substantially free from other materials. In one aspect, the term “isolated” refers to nucleic acid, such as DNA or RNA, or protein or polypeptide (e.g., an antibody or derivative thereof), or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

As used herein, the term “monoclonal antibody” refers to an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

The term “protein”, “peptide” and “polypeptide” are used interchangeably and in their broadest sense to refer to a compound of two or more subunit amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any aspect of this technology that is a polynucleotide encompasses both the double-stranded form and each of two complementary single-stranded forms known or predicted to make up the double-stranded form.

As used herein, the term “purified” does not require absolute purity; rather, it is intended as a relative term. Thus, for example, a purified nucleic acid, peptide, protein, biological complexes or other active compound is one that is isolated in whole or in part from proteins or other contaminants. Generally, substantially purified peptides, proteins, biological complexes, or other active compounds for use within the disclosure comprise more than 80% of all macromolecular species present in a preparation prior to admixture or formulation of the peptide, protein, biological complex or other active compound with a pharmaceutical carrier, excipient, buffer, absorption enhancing agent, stabilizer, preservative, adjuvant or other co-ingredient in a complete pharmaceutical formulation for therapeutic administration. More typically, the peptide, protein, biological complex or other active compound is purified to represent greater than 90%, often greater than 95% of all macromolecular species present in a purified preparation prior to admixture with other formulation ingredients. In other cases, the purified preparation may be essentially homogeneous, wherein other macromolecular species are not detectable by conventional techniques.

As used herein, the term “specific binding” means the contact between an antibody and an antigen with a binding affinity of at least 10⁻⁶ M. In certain aspects, antibodies bind with affinities of at least about 10⁻⁷ M, and preferably 10⁻⁸ M, 10⁻⁹ M, 10¹⁰ M, 10⁻¹¹ M, or 10⁻¹² M.

As used herein, the term “recombinant protein” refers to a polypeptide which is produced by recombinant DNA techniques, wherein generally, DNA encoding the polypeptide is inserted into a suitable expression vector which is in turn used to transform a host cell to produce the heterologous protein.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable.

As used herein, the term “overexpress” with respect to a cell, a tissue, or an organ expresses a protein to an amount that is greater than the amount that is produced in a control cell, a control issue, or an organ. A protein that is overexpressed may be endogenous to the host cell or exogenous to the host cell.

As used herein the term “linker sequence” relates to any amino acid sequence comprising from 1 to 10, or alternatively, 8 amino acids, or alternatively 6 amino acids, or alternatively 5 amino acids that may be repeated from 1 to 10, or alternatively to about 8, or alternatively to about 6, or alternatively about 5, or 4 or alternatively 3, or alternatively 2 times. For example, the linker may comprise up to 15 amino acid residues consisting of a pentapeptide repeated three times. In one aspect, the linker sequence is a (Glycine4Serine)3 flexible polypeptide linker comprising three copies of gly-gly-gly-gly-ser.

As used herein, the term “enhancer”, as used herein, denotes sequence elements that augment, improve or ameliorate transcription of a nucleic acid sequence irrespective of its location and orientation in relation to the nucleic acid sequence to be expressed. An enhancer may enhance transcription from a single promoter or simultaneously from more than one promoter. As long as this functionality of improving transcription is retained or substantially retained (e.g., at least 70%, at least 80%, at least 90% or at least 95% of wild-type activity, that is, activity of a full-length sequence), any truncated, mutated or otherwise modified variants of a wild-type enhancer sequence are also within the above definition.

As used herein, the term “WPRE” or “Woodchuck Hepatitis Virus (WHP) Post-transcriptional Regulatory Element” refers to a specific nucleotide fragment associated with this name and any other molecules that have analogous biological function that share at least 70%, or alternatively at least 80% amino acid sequence identity, preferably 90% sequence identity, more preferably at least 95% sequence identity with the WPRE sequence as shown herein. For example, WPRE refers to a region similar to the human hepatitis B virus posttranscriptional regulatory element (HBVPRE) present in the Woodchuck hepatitis virus genomic sequence (GenBank Accession No. J04514), and that the 592 nucleotides from position 1093 to 1684 of this genomic sequence correspond to the post-transcriptional regulatory region (Journal of Virology, Vol. 72, p. 5085-5092, 1998). The analysis using retroviral vectors revealed that WPRE inserted into the 3′-terminal untranslated region of a gene of interest increases the amount of protein produced by 5 to 8 folds. It has also been reported that the introduction of WPRE suppresses mRNA degradation (Journal of Virology, Vol. 73, p. 2886-2892, 1999). In a broad sense, elements such as WPRE that increase the efficiency of amino acid translation by stabilizing mRNAs are also thought to be enhancers.

### List of Abbreviations

CAR: chimeric antigen receptor
HLA: histocompatibility lymphocyte antigen
Ip: intraperitoneal
IRES: internal ribosomal entry site
MFI: mean fluorescence intensity
MOI: multiplicity of infection
PBMC: peripheral blood mononuclear cells
PBS: phosphate buffered saline
scFv: single chain variable fragment
WPRE: woodchuck hepatitis virus post-transcriptional regulatory element

The sequences associated with each of the above listed GenBank Accession Nos., UniProt Reference Nos., and references are herein incorporated by reference.

### MODES FOR CARRYING OUT THE DISCLOSURE

Due to the unprecedented results being recently obtained in B-cell lymphomas and leukemia's using autologous treatment with genetically engineered chimeric antigen receptor (CAR) T-cells (Maude, S. L. et al. (2014) New Engl. J. Med. 371:1507-1517; Porter, D. L. et al. (2011) New Engl. J. Med. 365:725-733), a number of laboratories have begun to apply this approach to solid tumors including ovarian cancer, prostate cancer, and pancreatic tumors. CAR modified T-cells combine the HLA-independent targeting specificity of a monoclonal antibody with the cytolytic activity, proliferation, and homing properties of activated T-cells, but do not respond to checkpoint suppression. Because of their ability to kill antigen expressing targets directly, CAR T-cells are highly toxic to any antigen positive cells or tissues making it a requirement to construct CARs with highly tumor specific antibodies. To date, CAR modified T-cells to human solid tumors have been constructed against the α-folate receptor, mesothelin, and MUC-CD, PSMA, and other targets but most have some off-target expression of antigen in normal tissues. These constructs have not shown the same exceptional results in patients emphasizing the need for additional studies to identify new targets and methods of CAR T-cell construction that can be used against solid tumors.

Thus, this disclosure provides antibodies specific to B7-H4 (or “anti-B7-H4”) and methods and compositions relating to the use and production thereof. In addition, this disclosure provides as a chimeric antigen receptor (CAR) comprising an antigen binding domain specific to B7-H4, that in some aspects, is the antigen binding domain of an anti-B7-H4 antibody and methods and compositions relating to the use and production thereof.

### Antibodies and Uses Thereof

I. Compositions

The general structure of antibodies is known in the art and will only be briefly summarized here. An immunoglobulin monomer comprises two heavy chains and two light chains connected by disulfide bonds. Each heavy chain is paired with one of the light chains to which it is directly bound via a disulfide bond. Each heavy chain comprises a constant region (which varies depending on the isotype of the antibody) and a variable region. The variable region comprises three hypervariable regions (or complementarity determining regions) which are designated CDRH1, CDRH2 and CDRH3 and which are supported within framework regions. Each light chain comprises a constant region and a variable region, with the variable region comprising three hypervariable regions (designated CDRL1, CDRL2 and CDRL3) supported by framework regions in an analogous manner to the variable region of the heavy chain.

The hypervariable regions of each pair of heavy and light chains mutually cooperate to provide an antigen binding site that is capable of binding a target antigen. The binding specificity of a pair of heavy and light chains is defined by the sequence of CDR1, CDR2 and CDR3 of the heavy and light chains. Thus once a set of CDR sequences (i.e., the sequence of CDR1, CDR2 and CDR3 for the heavy and light chains) is determined which gives rise to a particular binding specificity, the set of CDR sequences can, in principle, be inserted into the appropriate positions within any other antibody framework regions linked with any antibody constant regions in order to provide a different antibody with the same antigen binding specificity.

In one aspect, the present disclosure provides an isolated antibody comprising a heavy chain (HC) immunoglobulin variable domain sequence and a light chain (LC) immunoglobulin variable domain sequence, wherein the heavy chain and light chain immunoglobulin variable domain sequences form an antigen binding site that binds to an epitope of human B7-H4. In one aspect, the antibodies possess a binding affinity of at least 10⁻⁶ M. In certain aspects, antibodies bind with affinities of at least about 10⁻⁷ M, and preferably 10⁻⁸ M, 10⁻⁹ M, 10⁻¹⁰ M, 10⁻¹¹ M, or 10⁻¹² M.

In some embodiments, the heavy chain variable region comprises a CDRH1 sequence comprising, or alternatively consisting essentially of, or yet further consisting of, an amino acid sequence beginning with GXTF (SEQ ID NO: 1) followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus. In further embodiments, the CDRH1 sequence comprises, or alternatively consists essentially of, or yet further consisting of, an amino acid sequence beginning with any one of the following sequences: (i) GFTFSSFG (SEQ ID NO: 2), (ii) GFTFSSYG (SEQ ID NO: 3), (iii) GYTFTDY (SEQ ID NO: 4), or equivalents thereof, followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In some embodiments, the heavy chain variable region comprises a CDRH2 sequence comprising, or alternatively consisting essentially of, or yet further consisting of, an amino acid sequence beginning with ISSXXXT (SEQ ID NO: 5) followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus. In further embodiments, the CDRH2 sequence comprises, or alternatively consists essentially of, or yet further consists of, an amino acid sequence beginning with any one of the following sequences: (i) ISSGSSTL (SEQ ID NO: 6), (ii) ISSSNSTI (SEQ ID NO: 7), or equivalents thereof, followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In other embodiments, the heavy chain variable region comprises a CDRH2 sequence comprising, or alternatively consisting essentially of, or yet further consisting of, an amino acid sequence beginning with INPNNGGT (SEQ ID NO: 8) or an equivalent thereof, followed by by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In some embodiments, the heavy chain variable region comprises a CDRH3 sequence comprising, or alternatively consisting essentially of, or yet further consisting of, an amino acid sequence beginning with ARPXYY (SEQ ID NO: 9) followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus. In further embodiments, the CDRH3 sequence comprises, or alternatively consists essentially of, or yet further consisting of, an amino acid sequence beginning with any one of the following sequences: (i) ARPLYYYGSVMDY (SEQ ID NO: 10), (ii) ARPYYYGSSYDY (SEQ ID NO: 11), or equivalents thereof, followed by followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In some embodiments, the heavy chain variable region comprises, or alternatively consists essentially of, or yet further consists of, the polypeptide encoded by the below noted polynucleotide sequences: GAGGTGCAGCTGGAGGAGTCTGGGGGAGGCTTAGTGCAGCCTGGAGGGTCCCGG AAACTCTCCTGTGCAGCCTCTGGATTCACTTTCAGTAGCTTTGGAATGCACTGGG TTCGTCAGGCTCCAGAGAAGGGGCTGGAGTGGGTCGCATACATTAGTAGTGGCA GTAGTACCCTCCACTATGCAGACACAGTGAAGGGCCGATTCACCATCTCCAGAG ACAATCCCAAGAACACCCTGTTCCTGCAAATGAAACTACCCTCACTATGCTATGG ACTACTGGGGTCAAGGAACCTCAGTCACCGTCTCCTC (SEQ ID NO: 12) or an antigen binding fragment thereof or an equivalent of each thereof.

In some embodiments, the heavy chain variable region comprises, or alternatively consists essentially of, or yet further consists of, the amino acid sequence: EVQLEESGGGLVQPGGSRKLSCAASGFTFSSFGMHWVRQAPEKGLEWVAYISSGSST LHYADTVKGRFTISRDNPKNTLFLQMKLPSLCYGLLGSRNLSHRLL (SEQ ID NO: 13) or an antigen binding fragment thereof or an equivalent of each thereof.

In some embodiments, the heavy chain variable region comprises, or alternatively consists essentially of, or yet further consists of, the polypeptide encoded by the below noted polynucleotide sequences: GATGTGCAGCTGGTGGAGTCTGGGGGAGGTTTAGTGCAGCCTGGAGGGTCCCGG AAACTCTCCTGTGCAGCCTCTGGATTCACTTTCAGTAGCTATGGAATTCACTGGG TTCGTCAGGTTCCAGAGAAGGGGCTGGAGTGGGTCGCATTTATTAGTAGTAGCAA TTCTACCATCTACTATGCAGACACAGTGAAGGGCCGATTCACCATCTCCAGAGAC AATGCCGAGAACACCCTGTTCCTGCAAATGACCAGTCTAAGGTCTGAGGACACG GCCATGTATTACTGTGCAAGACCCCTTTACTACTATGGTAGCGTTATGGACTACT GGGGTCAAGGAACCTCTGTCACCGTCTCCTCA (SEQ ID NO: 14) or an antigen binding fragment thereof or an equivalent of each thereof.

In some embodiments, the heavy chain variable region comprises, or alternatively consists essentially of, or yet further consists of, the amino acid sequence: DVQLVESGGGLVQPGGSRKLSCAASGFTFSSYGIHWVRQVPEKGLEWVAFISSSNSTI YYADTVKGRFTISRDNAENTLFLQMTSLRSEDTAMYYCARPLYYYGSVMDYWGQG TSVTVSS (SEQ ID NO: 15) or an antigen binding fragment thereof or an equivalent of each thereof.

In some embodiments, the heavy chain variable region comprises, or alternatively consists essentially of, or yet further consists of, the polypeptide encoded by the below noted polynucleotide sequences: GAGGTCCAGCTGCAACAATCTGGACCTGAGCTGGTGAAGCCTGGGGCTTCAGTG AAGATATCCTGTAAGGCTTCTGGATACACGTTCACTGACTACTACATGAACTGGA TGAAGCAGAGCCATGGAAAGAGTCTTGAGTGGATTGGAGATATTAATCCTAACA ATGGTGGTACTAGCTACAACCAGAAGTTCAAGGGCAAGGCCACATTGACTGTAG ACAAGTCCTCCAGCACAGCCTACATGGAACTCCGCAGCCTGACATCTGAGGACT CTGCAGTCTATTACTGTGCAAGACCTTATTACTACGGTAGTAGCTACGACTACTG GGGCCAAGGCACCACTCTCACAGTCTCCTCA (SEQ ID NO: 16) or an antigen binding fragment thereof or an equivalent of each thereof.

In some embodiments, the heavy chain variable region comprises, or alternatively consists essentially of, or yet further consists of, the amino acid sequence: EVQLQQSGPELVKPGASVKISCKASGYTFTDYYMNWMKQSHGKSLEWIGDINPNNG GTSYNQKFKGKATLTVDKSSSTAYMELRSLTSEDSAVYYCARPYYYGSSYDYWGQ GTTLTVS (SEQ ID NO: 17) or an antigen binding fragment thereof or an equivalent of each thereof.

In some embodiments, the light chain variable region comprises a CDRL1 sequence comprising, or alternatively consisting essentially of, or yet further consisting of, an amino acid sequence beginning with QSIVHXNGTY (SEQ ID NO: 18) followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus. In further embodiments, the CDRL1 sequence comprises, or alternatively consists essentially of, or yet further consists of, an amino acid sequence beginning with any one of the following sequences: (i) QSIVHRNGNTY (SEQ ID NO: 19), (ii) QSIVHSNGNTY (SEQ ID NO: 20), or equivalents thereof, followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In other embodiments, the light chain variable region comprises a CDRL1 sequence comprising, or alternatively consisting essentially of, or yet further consisting of, an amino acid sequence beginning with ENIGSY (SEQ ID NO: 21) or an equivalent thereof, followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In some embodiments, the light chain variable region comprises a CDRL2 sequence comprising, or alternatively consisting essentially of, or yet further consisting of, an amino acid sequence beginning with KVS (SEQ ID NO: 22) followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In other embodiments, the light chain variable region comprises a CDRL2 sequence comprising, or alternatively consisting essentially of, or yet further consisting of, an amino acid sequence beginning with AAT (SEQ ID NO: 23) or an equivalent thereof, followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In some embodiments, the light chain variable region comprises a CDRL3 sequence comprising, or alternatively consisting essentially of, or yet further consisting of, an amino acid sequence beginning with FQGSXVPXT (SEQ ID NO: 24) followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus. In further embodiments, the CDRL1 sequence comprises, or alternatively consists essentially of, or yet further consists of, an amino acid sequence beginning with any one of the following sequences: (i) FQGSYVPPT (SEQ ID NO: 25), (ii) FQGSHVPLT (SEQ ID NO: 26), or equivalents thereof, followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In other embodiments, the light chain variable region comprises a CDRL3 sequence comprising, or alternatively consisting essentially of, or yet further consisting of, an amino acid sequence beginning with QHYYSTLVT (SEQ ID NO: 27) or an equivalent thereof, followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In some embodiments, the light chain variable region comprises, or alternatively consists essentially of, or yet further consists of, the polypeptide encoded by the polynucleotide sequence: GACATTGTGATCACCCAAACTCCACTCTCCCTGCCTGTCAGTCTTGGAGATCAAG CCTCCATCTCTTGCAGATCTAGTCAGAGCATTGTACATAGGAATGGAAACACCTA TTTAGAATGGTACTTGCAGCAACCAGGCCAGTCTCCAAAGCTCCTGATCTACAAA GTTTCCAACCGATTTTCTGGGGTCCCAGACAGGTTCAGTGGCAGTGGATCAGGGA CAGATTTCACACTCAAGATCAGCAGAGTGGAGGCTGAAGATCTGGGAGTTTATT ACTGCTTTCAAGGTTCATATGTTCCTCCGACGTTCGGTGGAGGCACCAAGCTGGA AATCAAA (SEQ ID NO: 28) or an antigen binding fragment thereof or an equivalent of each thereof.

In some embodiments, the light chain variable region comprises, or alternatively consists essentially of, or yet further consists of, the amino acid sequence: DIVITQTPLSLPVSLGDQASISCRSSQSIVHRNGNTYLEWYLQQPGQSPKLLIYKVSNR FSGVPDRFSGSGSGTDFTLKISRVEAEDLGVYYCFQGSYVPPTFGGGTKLEIK (SEQ ID NO: 29) or an antigen binding fragment thereof or an equivalent of each thereof.

In some embodiments, the light chain variable region comprises, or alternatively consists essentially of, or yet further consists of, the polypeptide encoded by the polynucleotide sequence: GATGTTTTGATGACCCAAACTCCACTCTCCCTGCCTGTCAGTCTTGGAGATCAAG CCTCCATCTCTTGCAGATCTAGTCAGAGCATTGTACATAGTAATGGAAACACCTA TTTAGAATGGTACCTGCAGAAACCAGGCCAGTCTCCAAAGCTCCTGATCTACAAA GTTTCCAACCGATTTTCTGGGGTCCCAGACAGGTTCAGTGGCAGTGGATCAGGGA CAGATTTCACACTCAAGATAAGTAGAGTGGAGGCTGAGGATCTGGGAGTTTATT ACTGCTTTCAAGGTTCACATGTTCCTCTCACGTTCGGTGCAGGGACCAAGCTGGA ACTGAAA (SEQ ID NO: 30) or an antigen binding fragment thereof or an equivalent of each thereof.

In some embodiments, the light chain variable region comprises, or alternatively consists essentially of, or yet further consists of, the amino acid sequence: DVLMTQTPLSLPVSLGDQASISCRSSQSIVHSNGNTYLEWYLQKPGQSPKLLIYKVSN RFSGVPDRFSGSGSGTDFTLKISRVEAEDLGVYYCFQGSHVPLTFGAGTKLELK (SEQ ID NO: 31) or an antigen binding fragment thereof or an equivalent of each thereof.

In some embodiments, the light chain variable region comprises, or alternatively consists essentially of, or yet further consists of, the polypeptide encoded by the polynucleotide sequence: GACATCCAGATGACTCAGTCTCCAGCTTCCCTGTCTGCATCTGTGGGAGAAACTG TCACCATCACATGTCGAGCAAGTGAAAATATTGGCAGTTATTTAGCATGGTATCA GCAGAAACAGGGAAAATCTCCTCAGCTCCTGGTCTATGCTGCAACACTCTTAGCA GATGGTGTGCCATCAAGGTTCAGTGGCAGTGGATCAGGCACACAGTTTTCTCTCA AGATCAACAGCCTGCAGTCTGAAGATGTTGCGAGATATTACTGTCAACATTATTA TAGTACTCTGGTCACGTTCGGTGCTGGGACCAAGCTGGAACTGAAA (SEQ ID NO: 32) or an antigen binding fragment thereof or an equivalent of each thereof.

In some embodiments, the light chain variable region comprises, or alternatively consists essentially of, or yet further consists of, the amino acid sequence: DIQMTQSPASLSASVGETVTITCRASENIGSYLAWYQQKQGKSPQLLVYAATLLADG VPSRFSGSGSGTQFSLKINSLQSEDVARYYCQHYYSTLVTFGAGTKLELK (SEQ ID NO: 33) or an antigen binding fragment thereof or an equivalent of each thereof.

In another aspect of the present technology, the isolated antibody includes one or more of the following characteristics:

(a) the light chain immunoglobulin variable domain sequence comprises one or more CDRs that are at least 85% identical to a CDR of a light chain variable domain of any of the disclosed light chain sequences;

(b) the heavy chain immunoglobulin variable domain sequence comprises one or more CDRs that are at least 85% identical to a CDR of a heavy chain variable domain of any of the disclosed heavy chain sequences;

(c) the light chain immunoglobulin variable domain sequence is at least 85% identical to a light chain variable domain of any of the disclosed light chain sequences;

(d) the HC immunoglobulin variable domain sequence is at least 85% identical to a heavy chain variable domain of any of the disclosed light chain sequences; and

(e) the antibody binds an epitope that overlaps with an epitope bound by any of the disclosed sequences.

Exemplary antibodies comprising the disclosed CDR sequences and heavy and light chain variable sequences are disclosed in Table 1 and Table 2, respectively.

TABLE 1

| ANTIBODY | CDRH1 | CDRH2 | CDRH3 | CDRL1 | CDRL2 | CDRL3 |
|---|---|---|---|---|---|---|
| B7H4 5F6 | SEQ ID | SEQ ID | n/a | SEQ ID | SEQ ID | SEQ ID |
| | NO: 2 | NO: 6 | | NO: 19 | NO: 22 | NO: 25 |
| B7H4 # 33- | SEQ ID | SEQ ID | SEQ ID | SEQ ID | SEQ ID | SEQ ID |
| 14 | NO: 3 | NO: 7 | NO: 10 | NO: 20 | NO: 22 | NO: 26 |
| B7H4 #36-1 | SEQ ID | SEQ ID | SEQ ID | SEQ ID | SEQ ID | SEQ ID |
| | NO: 4 | NO: 8 | NO: 11 | NO: 21 | NO: 23 | NO: 27 |

TABLE 2

| | Heavy Chain Variable | Light Chain Variable |
|---|---|---|
| ANTIBODY | Region | Region |
| B7H4 5F6 | SEQ ID NO: 13 | SEQ ID NO: 29 |
| B7H4 # 33-14 | SEQ ID NO: 15 | SEQ ID NO: 31 |
| B7H4 #36-1 | SEQ ID NO: 17 | SEQ ID NO: 33 |

In one aspect, the present disclosure provides an isolated antibody that is at least 85% identical to an antibody selected from the group consisting of B7H4 5F6, B7H4 #33-14, and B7H4 #36-1.

In a further aspect, the antibodies identified above possess a binding affinity of at least 10⁻⁶ M. In certain aspects, antibodies bind with affinities of at least about 10⁻⁷ M, and preferably 10⁻⁸ M, 10⁻⁹ M, 10⁻¹⁰ M, 10⁻¹¹ M. or 10⁻¹² M.

In one aspect, the present disclosure provides an isolated antibody comprising the CDRs of B7H4 5F6. In one aspect, the present disclosure provides an isolated antibody that is at least 85% identical to B7H4 5F6.

In one aspect, the present disclosure provides an isolated antibody comprising the CDRs of B7H4 #33-14. In one aspect, the present disclosure provides an isolated antibody that is at least 85% identical to B7H4 #33-14.

In one aspect, the present disclosure provides an isolated antibody comprising the CDRs of B7H4 #36-1. In one aspect, the present disclosure provides an isolated antibody that is at least 85% identical to B7H4 #36-1.

In some aspects of the antibodies provided herein, the HC variable domain sequence comprises a variable domain sequence of B7H4 5F6 and the LC variable domain sequence comprises a variable domain sequence of B7H4 5F6.

In some aspects of the antibodies provided herein, the HC variable domain sequence comprises a variable domain sequence of B7H4 #33-14 and the LC variable domain sequence comprises a variable domain sequence of B7H4 #33-14.

In some aspects of the antibodies provided herein, the HC variable domain sequence comprises a variable domain sequence of B7H4 #36-1 and the LC variable domain sequence comprises a variable domain sequence of B7H4 #36-1.

In some of the aspects of the antibodies provided herein, the antibody binds human B7-H4 with a dissociation constant (K_{D}) of less than 10⁻⁴ M, 10⁻⁵ M, 10⁻⁶ M, 10⁻⁷ M, 10⁻⁸ M, 10⁻⁹M, 10⁻¹⁰ M, 10⁻¹¹ M, or 10⁻¹²M. In some of the aspects of the antibodies provided herein, the antigen binding site specifically binds to human B7-H4.

In some of the aspects of the antibodies provided herein, the antibody is soluble Fab.

In some of the aspects of the antibodies provided herein, the HC and LC variable domain sequences are components of the same polypeptide chain. In some of the aspects of the antibodies provided herein, the HC and LC variable domain sequences are components of different polypeptide chains.

In some of the aspects of the antibodies provided herein, the antibody is a full-length antibody.

In some of the aspects of the antibodies provided herein, the antibody is a monoclonal antibody.

In some of the aspects of the antibodies provided herein, the antibody is chimeric or humanized.

In some of the aspects of the antibodies provided herein, the antibody is selected from the group consisting of Fab, F(ab)′2, Fab′, scFᵥ, and Fᵥ.

In some of the aspects of the antibodies provided herein, the antibody comprises an Fc domain. In some of the aspects of the antibodies provided herein, the antibody is a rabbit antibody. In some of the aspects of the antibodies provided herein, the antibody is a human or humanized antibody or is non-immunogenic in a human.

In some of the aspects of the antibodies provided herein, the antibody comprises a human antibody framework region.

In other aspects, one or more amino acid residues in a CDR of the antibodies provided herein are substituted with another amino acid. The substitution may be “conservative” in the sense of being a substitution within the same family of amino acids. The naturally occurring amino acids may be divided into the following four families and conservative substitutions will take place within those families.

1) Amino acids with basic side chains: lysine, arginine, histidine.

2) Amino acids with acidic side chains: aspartic acid, glutamic acid

3) Amino acids with uncharged polar side chains: asparagine, glutamine, serine, threonine, tyrosine.

4) Amino acids with nonpolar side chains: glycine, alanine, valine, leucine, isoleucine, proline, phenylalanine, methionine, tryptophan, cysteine.

In another aspect, one or more amino acid residues are added to or deleted from one or more CDRs of an antibody. Such additions or deletions occur at the N or C termini of the CDR or at a position within the CDR.

By varying the amino acid sequence of the CDRs of an antibody by addition, deletion or substitution of amino acids, various effects such as increased binding affinity for the target antigen may be obtained.

It is to be appreciated that antibodies of the present disclosure comprising such varied CDR sequences still bind B7-H4 with similar specificity and sensitivity profiles as the disclosed antibodies. This may be tested by way of the binding assays.

The constant regions of antibodies may also be varied. For example, antibodies may be provided with Fc regions of any isotype: IgA (IgA1, IgA2), IgD, IgE, IgG (IgG1, IgG2, IgG3, IgG4) or IgM. Non-limiting examples of constant region sequences include:

Human IgD constant region, Uniprot: P01880
SEQ ID NO: 34
APTKAPDVFPIISGCRHPKDNSPVVLACLITGYHPTSVTVTWYMGTQSQPQ
RTFPEIQRRDSYYMTSSQLSTPLQQWRQGEYKCVVQHTASKSKKEIFRWPE
SPKAQASSVPTAQPQAEGSLAKATTAPATTRNTGRGGEEKKKEKEKEEQEE
RETKTPECPSHTQPLGVYLLTPAVQDLWLRDKATFTCFVVGSDLKDAHLTW
EVAGKVPTGGVEEGLLERHSNGSQSQHSRLTLPRSLWNAGTSVTCTLNHPS
LPPQRLMALREPAAQAPVKLSLNLLASSDPPEAASWLLCEVSGFSPPNILL
MWLEDQREVNTSGFAPARPPPQPGSTTFWAWSVLRVPAPPSPQPATYTCVV
SHEDSRTLLNASRSLEVSYVTDHGPMK
Human IgG1 constant region, Uniprot: P01857
SEQ ID NO: 35
ASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYKC
KVSNKALPAPIEKTISKAKGQPREPQVYTLPPSRDELTKNQVSLTCLVKGF
YPSDIAVEWESNGQPENNYKTIPPVLDSDGSFFLYSKLTVDKSRWQQGNVF
SCSVMHEALHNHYTQKSLSLSPGK
Human IgG2 constant region, Uniprot: P01859
SEQ ID NO: 36
ASTKGPSVFPLAPCSRSTSESTAALGCLVKDYFPEPVTVSWNSGALTSGVH
TFPAVLQSSGLYSLSSVVTVPSSNFGTQTYTCNVDHKPSNTKVDKPVERKC
CVECPPCPAPPVAGPSVFLFPPKPKDTLMLSRTPEVTCVVVDVSHEDPEVQ
FNWYVDGVEVHNAKTKPREEQFNSTFRVVSVLTVVHQDWLNGKEYKCKVSN
KGLPAPIEKTISKTKGQPREPQVYTLPPSREEMTKNQVSLTCLVKGFYPSD
ISVEWESNGQPENNYKTTPPMLDSDGSFFLYSKLTVDKSRWQQGNVFSCSV
MHEALHNHYTQKSLSLSPGK
Human IgG3 constant region, Uniprot: P01860
SEQ ID NO: 37
ASTKGPSVFPLAPCSRSTSGGTAALGCLVKDYFPEPVTVSWNSGALTSGVH
TFPAVLQSSGLYSLSSVVTVPSSSLGTQTYTCNVNHKPSNTKVDKRVELKT
PLGDTTHTCPRCPEPKSCDTPPPCPRCPEPKSCDTPPPCPRCPEPKSCDTP
PPCPRCPAPELLGGPSVFLFPPKPKDTLMISRTPEVTCVVVDVSHEDPEVQ
FKWYVDGVEVHNAKTKPREEQYNSTFRVVSVLTVLHQDWLNGKEYKCKVSN
KALPAPIEKTISKTKGQPREPQVYTLPPSREEMTKNQVSLTCLVKGFYPSD
IAVEWESSGQPENNYNTTPPMLDSDGSFFLYSKLTVDKSRWQQGNIFSCSV
MHEALHNRFTQKSLSLSPGK
Human IgM constant region, Uniprot: P01871
SEQ ID NO: 38
GSASAPTLFPLVSCENSPSDTSSVAVGCLAQDFLPDSITLSWKYKNNSDIS
STRGFPSVLRGGKYAATSQVLLPSKDVMQGTDEHVVCKVQHPNGNKEKNVP
LPVIAELPPKVSVFVPPRDGFFGNPRKSKLICQATGFSPRQIQVSWLREGK
QVGSGVTTDQVQAEAKESGPTTYKVTSTLTIKESDWLGQSMFTCRVDHRGL
TFQQNASSMCVPDQDTAIRVFAIPPSFASIFLTKSTKLTCLVTDLTTYDSV
TISWTRQNGEAVKTHTNISESHPNATFSAVGEASICEDDWNSGERFTCTVT
HTDLPSPLKQTISRPKGVALHRPDVYLLPPAREQLNLRESATITCLVTGFS
PADVFVQWMQRGQPLSPEKYVTSAPMPEPQAPGRYFAHSILTVSEEEWNTG
ETYTCVAHEALPNRVTERTVDKSTGKPTLNNVSLVMSDTAGTCY
Human IgG4 constant region, Uniprot: P01861
SEQ ID NO: 39
ASTKGPSVFPLAPCSRSTSESTAALGCLVKDYFPEPVTVSWNSGALTSGVH
TFPAVLQSSGLYSLSSVVTVPSSSLGTKTYTCNVDHKPSNTKVDKRVESKY
GPPCPSCPAPEFLGGPSVFLFPPKPKDTLMISRTPEVTCVVVDVSQEDPEV
QFNWYVDGVEVHNAKTKPREEQFNSTYRVVSVLTVLHQDWLNGKEYKCKVS
NKGLPSSIEKTISKAKGQPREPQVYTLPPSQEEMTKNQVSLTCLVKGFYPS
DIAVEWESNGQPENNYKTTPPVLDSDGSFFLYSRLTVDKSRWQEGNVFSCS
VMHEALHNHYTQKSLSLSLGK
Human IgAI constant region, Uniprot: P01876
SEQ ID NO: 40
ASPTSPKVFPLSLCSTQPDGNVVIACLVQGFFPQEPLSVTWSESGQGVTAR
NFPPSQDASGDLYTTSSQLTLPATQCLAGKSVTCHVKHYTNPSQDVTVPCP
VPSTPPTPSPSTPPTPSPSCCHPRLSLHRPALEDLLLGSEANLTCTLTGLR
DASGVTFTWTPSSGKSAVQGPPERDLCGCYSVSSVLPGCAEPWNHGKTFTC
TAAYPESKTPLTATLSKSGNTFRPEVHLLPPPSEELALNELVTLTCLARGF
SPKDVLVRWLQGSQELPREKYLTWASRQEPSQGTTTFAVTSILRVAAEDWK
KGDTFSCMVGHEALPLAFTQKTIDRLAGKPTHVNVSVVMAEVDGTCY
Human IgA2 constant region, Uniprot: P01877
SEQ ID NO: 41
ASPTSPKVFPLSLDSTPQDGNVVVACLVQGFFPQEPLSVTWSESGQNVTAR
NFPPSQDASGDLYTTSSQLTLPATQCPDGKSVTCHVKHYTNPSQDVTVPCP
VPPPPPCCHPRLSLHRPALEDLLLGSEANLTCTLTGLRDASGATFTWTPSS
GKSAVQGPPERDLCGCYSVSSVLPGCAQPWNHGETFTCTAAHPELKTPLTA
NITKSGNTFRPEVHLLPPPSEELALNELVTLTCLARGFSPKDVLVRWLQGS
QELPREKYLTWASRQEPSQGTTTFAVTSILRVAAEDWKKGDTFSCMVGHEA
LPLAFTQKTIDRMAGKFTHVNVSVVMAEVDGTCY
Human Ig kappa constant region, Uniprot: P01834
SEQ ID NO: 42
TVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFN
RGEC

In some aspects, the antibodies comprise a heavy chain constant region that is at least 80% identical to any one of SEQ ID NOs: 12 to 17.

In some aspects, the antibodies comprise a light chain constant region that is at least 80% identical to any one of SEQ ID NOs: 28 to 33.

In some aspects of the antibodies provided herein, the antibody binds to the epitope bound by B7H4 5F6, B7H4 #33-14, and B7H4 #36-1 antibodies.

In some aspects of the antibodies provided herein, the B7-H4-specific antibody competes for binding to human B7-H4 with B7H4 5F6, B7H4 #33-14, and B7H4 #36-1.

In some aspects of the antibodies provided herein, the antibody contains structural modifications to facilitate rapid binding and cell uptake and/or slow release. In some aspects, the B7-H4 antibody contains a deletion in the CH2 constant heavy chain region of the antibody to facilitate rapid binding and cell uptake and/or slow release. In some aspects, a Fab fragment is used to facilitate rapid binding and cell uptake and/or slow release. In some aspects, a F(ab)′2 fragment is used to facilitate rapid binding and cell uptake and/or slow release.

The antibodies, fragments, and equivalents thereof can be combined with a carrier, e.g., a pharmaceutically acceptable carrier or other agents to provide a formulation for use and/or storage.

Further provided is an isolated polypeptide comprising, or alternatively consisting essentially of, or yet further consisting of, the amino acid sequence of B7-H4, an equivalent thereof or a fragment thereof, that are useful to generate antibodies that bind to B7-H4, as well as isolated polynucleotides that encode them. In one aspect, the isolated polypeptides or polynucleotides further comprise a label or a selection marker, and/or contiguous polypeptide sequences (e.g., keyhole limpet haemocyanin (KLH) carrier protein) or in the case of polynucleotides, polynucleotides encoding the sequence, operatively coupled to polypeptide or polynucleotide. The polypeptides or polynucleotides can be combined with various carriers, e.g., phosphate buffered saline. Further provided are host cells, e.g., prokaryotic or eukaryotic cells, e.g., bacteria, yeast, mammalian (rat, simian, hamster, or human), comprising the isolated polypeptides or polynucleotides. The host cells can be combined with a carrier.

II. Processes for Preparing Compositions

Antibodies, their manufacture and uses are well known and disclosed in, for example, Harlow, E. and Lane, D., Antibodies: A Laboratory Manual, Cold Spring Harbor Laboratory Press, Cold Spring Harbor, N.Y., 1999. The antibodies may be generated using standard methods known in the art. Examples of antibodies include (but are not limited to) monoclonal, single chain, and functional fragments of antibodies.

Antibodies may be produced in a range of hosts, for example goats, rabbits, rats, mice, humans, and others. They may be immunized by injection with a target antigen or a fragment or oligopeptide thereof which has immunogenic properties, such as a C-terminal fragment of B7-H4 or an isolated polypeptide. Depending on the host species, various adjuvants may be added and used to increase an immunological response. Such adjuvants include, but are not limited to, Freund's, mineral gels such as aluminum hydroxide, and surface active substances such as lysolecithin, pluronic polyols, polyanions, peptides, oil emulsions, keyhole limpet hemocyanin, and dinitrophenol. Among adjuvants used in humans, BCG (Bacille Calmette-Guerin) and Corynebacterium parvum are particularly useful. This this disclosure also provides the isolated polypeptide and an adjuvant.

In certain aspects, the antibodies of the present disclosure are polyclonal, i.e., a mixture of plural types of anti-B7-H4 antibodies having different amino acid sequences. In one aspect, the polyclonal antibody comprises a mixture of plural types of anti-B7-H4 antibodies having different CDRs. As such, a mixture of cells which produce different antibodies is cultured, and an antibody purified from the resulting culture can be used (see WO 2004/061104).

Monoclonal Antibody Production.

Monoclonal antibodies to B7-H4 may be prepared using any technique which provides for the production of antibody molecules by continuous cell lines in culture. Such techniques include, but are not limited to, the hybridoma technique (see, e.g., Kohler & Milstein, Nature 256: 495-497 (1975)); the trioma technique; the human B-cell hybridoma technique (see, e.g., Kozbor, et al., Immunol. Today 4: 72 (1983)) and the EBV hybridoma technique to produce human monoclonal antibodies (see, e.g., Cole, et al., in: MONOCLONAL ANTIBODIES AND CANCER THERAPY, Alan R. Liss, Inc., pp. 77-96 (1985)). Human monoclonal antibodies can be utilized in the practice of the present technology and can be produced by using human hybridomas (see, e.g., Cote, et al., Proc. Natl. Acad. Sci. 80: 2026-2030 (1983)) or by transforming human B-cells with Epstein Barr Virus in vitro (see, e.g., Cole, et al., in: MONOCLONAL ANTIBODIES AND CANCER THERAPY, Alan R. Liss, Inc., pp. 77-96 (1985)). For example, a population of nucleic acids that encode regions of antibodies can be isolated. PCR utilizing primers derived from sequences encoding conserved regions of antibodies is used to amplify sequences encoding portions of antibodies from the population and then reconstruct DNAs encoding antibodies or fragments thereof, such as variable domains, from the amplified sequences. Such amplified sequences also can be fused to DNAs encoding other proteins—e.g., a bacteriophage coat, or a bacterial cell surface protein—for expression and display of the fusion polypeptides on phage or bacteria. Amplified sequences can then be expressed and further selected or isolated based, e.g., on the affinity of the expressed antibody or fragment thereof for an antigen or epitope present on the B7-H4 polypeptide. Alternatively, hybridomas expressing anti-B7-H4 monoclonal antibodies can be prepared by immunizing a subject, e.g., with an isolated polypeptide comprising, or alternatively consisting essentially of, or yet further consisting of, the amino acid sequence of B7-H4 or a fragment thereof, and then isolating hybridomas from the subject's spleen using routine methods. See, e.g., Milstein et al., (Galfre and Milstein, Methods Enzymol 73: 3-46 (1981)). Screening the hybridomas using standard methods will produce monoclonal antibodies of varying specificity (i.e., for different epitopes) and affinity. A selected monoclonal antibody with the desired properties, e.g., B7-H4 binding, can be (i) used as expressed by the hybridoma, (ii) bound to a molecule such as polyethylene glycol (PEG) to alter its properties, or (iii) a cDNA encoding the monoclonal antibody can be isolated, sequenced and manipulated in various ways. In one aspect, the anti-B7-H4 monoclonal antibody is produced by a hybridoma which includes a B cell obtained from a transgenic non-human animal, e.g., a transgenic mouse, having a genome comprising a human heavy chain transgene and a light chain transgene fused to an immortalized cell. Hybridoma techniques include those known in the art and taught in Harlow et al., Antibodies: A Laboratory Manual Cold Spring Harbor Laboratory, Cold Spring Harbor, N.Y., 349 (1988); Hammerling et al., Monoclonal Antibodies And T-Cell Hybridomas, 563-681 (1981).

Phage Display Technique.

As noted above, the antibodies of the present disclosure can be produced through the application of recombinant DNA and phage display technology. For example, anti-B7-H4 antibodies, can be prepared using various phage display methods known in the art. In phage display methods, functional antibody domains are displayed on the surface of a phage particle which carries polynucleotide sequences encoding them. Phage with a desired binding property is selected from a repertoire or combinatorial antibody library (e.g., human or murine) by selecting directly with an antigen, typically an antigen bound or captured to a solid surface or bead. Phage used in these methods are typically filamentous phage including fd and M13 with Fab, Fᵥ or disulfide stabilized Fᵥ antibody domains are recombinantly fused to either the phage gene III or gene VIII protein. In addition, methods can be adapted for the construction of Fab expression libraries (see, e.g., Huse, et al., Science 246: 1275-1281, 1989) to allow rapid and effective identification of monoclonal Fab fragments with the desired specificity for a B7-H4 polypeptide, e.g., a polypeptide or derivatives, fragments, analogs or homologs thereof. Other examples of phage display methods that can be used to make the isolated antibodies of the present disclosure include those disclosed in Huston et al., Proc. Natl. Acad. Sci. U.S.A., 85: 5879-5883 (1988); Chaudhary et al., Proc. Natl. Acad. Sci. U.S.A., 87: 1066-1070 (1990); Brinkman et al., J. Immunol. Methods 182: 41-50 (1995); Ames et al., J. Immunol. Methods 184: 177-186 (1995); Kettleborough et al., Eur. J. Immunol. 24: 952-958 (1994); Persic et al., Gene 187: 9-18 (1997); Burton et al., Advances in Immunology 57: 191-280 (1994); PCT/GB91/01134; WO 90/02809; WO 91/10737; WO 92/01047; WO 92/18619; WO 93/11236; WO 95/15982; WO 95/20401; WO 96/06213; WO 92/01047 (Medical Research Council et al.); WO 97/08320 (Morphosys); WO 92/01047 (CAT/MRC); WO 91/17271 (Affymax); and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727 and 5,733,743.

Methods useful for displaying polypeptides on the surface of bacteriophage particles by attaching the polypeptides via disulfide bonds have been described by Lohning, U.S. Pat. No. 6,753,136. As described in the above references, after phage selection, the antibody coding regions from the phage can be isolated and used to generate whole antibodies, including human antibodies, or any other desired antigen binding fragment, and expressed in any desired host including mammalian cells, insect cells, plant cells, yeast, and bacteria. For example, techniques to recombinantly produce Fab, Fab′ and F(ab′)₂ fragments can also be employed using methods known in the art such as those disclosed in WO 92/22324; Mullinax et al., BioTechniques 12: 864-869 (1992); Sawai et al., AJRI 34: 26-34 (1995); and Better et al., Science 240: 1041-1043 (1988).

Generally, hybrid antibodies or hybrid antibody fragments that are cloned into a display vector can be selected against the appropriate antigen in order to identify variants that maintained good binding activity, because the antibody or antibody fragment will be present on the surface of the phage or phagemid particle. See e.g. Barbas III et al., Phage Display, A Laboratory Manual (Cold Spring Harbor Laboratory Press, Cold Spring Harbor, N.Y., 2001). However, other vector formats could be used for this process, such as cloning the antibody fragment library into a lytic phage vector (modified T7 or Lambda Zap systems) for selection and/or screening.

Alternate Methods of Antibody Production.

Antibodies may also be produced by inducing in vivo production in the lymphocyte population or by screening recombinant immunoglobulin libraries or panels of highly specific binding reagents (Orlandi et al., PNAS 86: 3833-3837 (1989); Winter, G. et al., Nature, 349: 293-299 (1991)).

Alternatively, techniques for the production of single chain antibodies may be used. Single chain antibodies (scFᵥs) comprise a heavy chain variable region and a light chain variable region connected with a linker peptide (typically around 5 to 25 amino acids in length). In the scFᵥ, the variable regions of the heavy chain and the light chain may be derived from the same antibody or different antibodies. scFᵥs may be synthesized using recombinant techniques, for example by expression of a vector encoding the scFᵥ in a host organism such as E. coli. DNA encoding scFᵥ can be obtained by performing amplification using a partial DNA encoding the entire or a desired amino acid sequence of a DNA selected from a DNA encoding the heavy chain or the variable region of the heavy chain of the above-mentioned antibody and a DNA encoding the light chain or the variable region of the light chain thereof as a template, by PCR using a primer pair that defines both ends thereof, and further performing amplification combining a DNA encoding a polypeptide linker portion and a primer pair that defines both ends thereof, so as to ligate both ends of the linker to the heavy chain and the light chain, respectively. An expression vector containing the DNA encoding scFᵥ and a host transformed by the expression vector can be obtained according to conventional methods known in the art.

Antigen binding fragments may also be generated, for example the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed to allow rapid and easy identification of monoclonal Fab fragments with the desired specificity (Huse et al., Science, 256: 1275-1281 (1989)).

Antibody Modifications.

The antibodies of the present disclosure may be multimerized to increase the affinity for an antigen. The antibody to be multimerized may be one type of antibody or a plurality of antibodies which recognize a plurality of epitopes of the same antigen. As a method of multimerization of the antibody, binding of the IgG CH3 domain to two scFᵥ molecules, binding to streptavidin, introduction of a helix-turn-helix motif and the like can be exemplified.

The antibody compositions disclosed herein may be in the form of a conjugate formed between any of these antibodies and another agent (immunoconjugate). In one aspect, the antibodies disclosed herein are conjugated to radioactive material. In another aspect, the antibodies disclosed herein can be bound to various types of molecules such as polyethylene glycol (PEG).

Antibody Screening.

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between B7-H4, or any fragment or oligopeptide thereof and its specific antibody. A two-site, monoclonal-based immunoassay utilizing monoclonal antibodies specific to two non-interfering B7-H4 epitopes may be used, but a competitive binding assay may also be employed (Maddox et al., J. Erp. Med., 158: 1211-1216 (1983)).

Antibody Purification.

The antibodies disclosed herein can be purified to homogeneity. The separation and purification of the antibodies can be performed by employing conventional protein separation and purification methods.

By way of example only, the antibody can be separated and purified by appropriately selecting and combining use of chromatography columns, filters, ultrafiltration. salt precipitation, dialysis, preparative polyacrylamide gel electrophoresis, isoelectric focusing electrophoresis, and the like. Strategies for Protein Purification and Characterization: A Laboratory Course Manual, Daniel R. Marshak et al. eds., Cold Spring Harbor Laboratory Press (1996); Antibodies: A Laboratory Manual. Ed Harlow and David Lane, Cold Spring Harbor Laboratory (1988).

Examples of chromatography include affinity chromatography, ion exchange chromatography, hydrophobic chromatography, gel filtration chromatography, reverse phase chromatography, and adsorption chromatography. In one aspect, chromatography can be performed by employing liquid chromatography such as HPLC or FPLC.

In one aspect, a Protein A column or a Protein G column may be used in affinity chromatography. Other exemplary columns include a Protein A column, Hyper D, POROS, Sepharose F. F. (Pharmacia) and the like.

III. Methods of Use

General.

The antibodies disclosed herein are useful in methods known in the art relating to the localization and/or quantitation of a B7-H4 polypeptide (e.g., for use in measuring levels of the B7-H4 polypeptide within appropriate physiological samples, for use in diagnostic methods, for use in imaging the polypeptide, and the like). The antibodies disclosed herein are useful in isolating a B7-H4 polypeptide by standard techniques, such as affinity chromatography or immunoprecipitation. A B7-H4 antibody disclosed herein can facilitate the purification of natural B7-H4 polypeptides from biological samples, e.g., mammalian sera or cells as well as recombinantly-produced B7-H4 polypeptides expressed in a host system. Moreover, B7-H4 antibody can be used to detect a B7-H4 polypeptide (e.g., in plasma, a cellular lysate or cell supernatant) in order to evaluate the abundance and pattern of expression of the polypeptide. The B7-H4 antibodies disclosed herein can be used diagnostically to monitor B7-H4 levels in tissue as part of a clinical testing procedure, e.g., to determine the efficacy of a given treatment regimen. The detection can be facilitated by coupling (i.e., physically linking) the B7-H4 antibodies disclosed herein to a detectable substance.

In another aspect, provided herein is a composition comprising an antibody or antigen binding fragment as disclosed herein bound to a peptide comprising, for example, a human B7-H4 protein or a fragment thereof. In one aspect, the peptide is associated with a cell. For example, the composition may comprise a disaggregated cell sample labeled with an antibody or antibody fragment as disclosed herein, which composition is useful in, for example, affinity chromatography methods for isolating cells or for flow cytometry-based cellular analysis or cell sorting. As another example, the composition may comprise a fixed tissue sample or cell smear labeled with an antibody or antibody fragment as disclosed herein, which composition is useful in, for example, immunohistochemistry or cytology analysis. In another aspect, the antibody or the antibody fragment is bound to a solid support, which is useful in, for example: ELISAs; affinity chromatography or immunoprecipitation methods for isolating B7-H4 proteins or fragments thereof, B7-H4-positive cells, or complexes containing B7-H4 and other cellular components. In another aspect, the peptide is bound to a solid support. For example, the peptide may be bound to the solid support via a secondary antibody specific for the peptide, which is useful in, for example, sandwich ELISAs. As another example, the peptide may be bound to a chromatography column, which is useful in, for example, isolation or purification of antibodies according to the present technology. In another aspect, the peptide is disposed in a solution, such as a lysis solution or a solution containing a sub-cellular fraction of a fractionated cell, which is useful in, for example, ELISAs and affinity chromatography or immunoprecipitation methods of isolating B7-H4 proteins or fragments thereof or complexes containing B7-H4 and other cellular components. In another aspect, the peptide is associated with a matrix, such as, for example, a gel electrophoresis gel or a matrix commonly used for western blotting (such as membranes made of nitrocellulose or polyvinylidene difluoride), which compositions are useful for electrophoretic and/or immunoblotting techniques, such as Western blotting.

Detection of B7-H4 Polypeptide.

An exemplary method for detecting the level of B7-H4 polypeptides in a biological sample involves obtaining a biological sample from a subject and contacting the biological sample with a B7-H4 antibody disclosed herein which is capable of detecting the B7-H4 polypeptides.

In one aspect, the B7-H4 antibodies B7H4 5F6, B7H4 #33-14, or B7H4 #36-1, or fragments thereof are detectably labeled. The term “labeled”, with regard to the antibody is intended to encompass direct labeling of the antibody by coupling (i.e., physically linking) a detectable substance to the antibody, as well as indirect labeling of the antibody by reactivity with another compound that is directly labeled. Non-limiting examples of indirect labeling include detection of a primary antibody using a fluorescently-labeled secondary antibody and end-labeling of a DNA probe with biotin such that it can be detected with fluorescently-labeled streptavidin.

The detection method of the present disclosure can be used to detect expression levels of B7-H4 polypeptides in a biological sample in vitro as well as in vivo. In vitro techniques for detection of B7-H4 polypeptides include enzyme linked immunosorbent assays (ELISAs), Western blots, flow cytometry, immunoprecipitations, radioimmunoassay, and immunofluorescence (e.g., IHC). Furthermore, in vivo techniques for detection of B7-H4 polypeptides include introducing into a subject a labeled anti-B7-H4 antibody. By way of example only, the antibody can be labeled with a radioactive marker whose presence and location in a subject can be detected by standard imaging techniques. In one aspect, the biological sample contains polypeptide molecules from the test subject.

Immunoassay and Imaging.

A B7-H4 antibody disclosed herein can be used to assay B7-H4 polypeptide levels in a biological sample (e.g. human tumor sample) using antibody-based techniques. For example, protein expression in tissues can be studied with classical immunohistochemical (IHC) staining methods. Jalkanen, M. et al., J. Cell. Biol. 101: 976-985 (1985); Jalkanen, M. et al., J. Cell. Biol. 105: 3087-3096 (1987). Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase, and radioisotopes or other radioactive agents, such as iodine (¹²⁵I, ¹²¹I, ¹³¹I), carbon (¹⁴C), sulfur (³⁵S), tritium (³H), indium (¹¹²In), and technetium (⁹⁹mTc), and fluorescent labels, such as fluorescein and rhodamine, and biotin.

In addition to assaying B7-H4 polypeptide levels in a biological sample, B7-H4 polypeptide levels can also be detected in vivo by imaging. Labels that can be incorporated with anti-B7-H4 antibodies for in vivo imaging of B7-H4 polypeptide levels include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which can be incorporated into the B7-H4 antibody by labeling of nutrients for the relevant scFᵥ clone.

A B7-H4 antibody which has been labeled with an appropriate detectable imaging moiety, such as a radioisotope (e.g., ¹³¹I, ¹¹²In, ⁹⁹mTc), a radio-opaque substance, or a material detectable by nuclear magnetic resonance, is introduced (e.g., parenterally, subcutaneously, or intraperitoneally) into the subject. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ⁹⁹mTc. The labeled B7-H4 antibody will then preferentially accumulate at the location of cells which contain the specific target polypeptide. For example, in vivo tumor imaging is described in S. W. Burchiel et al., Tumor Imaging: The Radiochemical Detection of Cancer 13 (1982).

In some aspects, B7-H4 antibodies containing structural modifications that facilitate rapid binding and cell uptake and/or slow release are useful in in vivo imaging detection methods. In some aspects, the B7-H4 antibody contains a deletion in the CH2 constant heavy chain region of the antibody to facilitate rapid binding and cell uptake and/or slow release. In some aspects, a Fab fragment is used to facilitate rapid binding and cell uptake and/or slow release. In some aspects, a F(ab)′2 fragment is used to facilitate rapid binding and cell uptake and/or slow release.

Diagnostic Uses of B7-H4 Antibodies.

The B7-H4 antibody compositions disclosed herein are useful in diagnostic and prognostic methods. As such, the present disclosure provides methods for using the antibodies disclosed herein in the diagnosis of B7-H4-related medical conditions in a subject. Antibodies disclosed herein may be selected such that they have a high level of epitope binding specificity and high binding affinity to the B7-H4 polypeptide. In general, the higher the binding affinity of an antibody, the more stringent wash conditions can be performed in an immunoassay to remove nonspecifically bound material without removing the target polypeptide. Accordingly, B7-H4 antibodies of the present technology useful in diagnostic assays usually have binding affinities of at least 10⁻⁶, 10⁻⁷, 10⁻⁸, 10⁻⁹, 10⁻¹⁰, 10⁻¹¹, or 10⁻¹² M. In certain aspects, B7-H4 antibodies used as diagnostic reagents have a sufficient kinetic on-rate to reach equilibrium under standard conditions in at least 12 hours, at least 5 hours, at least 1 hour, or at least 30 minutes.

Some methods of the present technology employ polyclonal preparations of anti-B7-H4 antibodies and polyclonal anti-B7-H4 antibody compositions as diagnostic reagents, and other methods employ monoclonal isolates. In methods employing polyclonal human anti-B7-H4 antibodies prepared in accordance with the methods described above, the preparation typically contains an assortment of B7-H4 antibodies, e.g., antibodies, with different epitope specificities to the target polypeptide. The monoclonal anti-B7-H4 antibodies of the present disclosure are useful for detecting a single antigen in the presence or potential presence of closely related antigens.

The B7-H4 antibodies of the present disclosure can be used as diagnostic reagents for any kind of biological sample. In one aspect, the B7-H4 antibodies disclosed herein are useful as diagnostic reagents for human biological samples. B7-H4 antibodies can be used to detect B7-H4 polypeptides in a variety of standard assay formats. Such formats include immunoprecipitation, Western blotting, ELISA, radioimmunoassay, flow cytometry, IHC and immunometric assays. See Harlow & Lane, Antibodies, A Laboratory Manual (Cold Spring Harbor Publications, New York, 1988); U.S. Pat. Nos. 3,791,932; 3,839,153; 3,850,752; 3,879,262; 4,034,074, 3,791,932; 3,817,837; 3,839,153; 3,850,752; 3,850,578; 3,853,987; 3,867,517; 3,879,262; 3,901,654; 3,935,074; 3,984,533; 3,996,345; 4,034,074; and 4,098,876. Biological samples can be obtained from any tissue (including biopsies), cell or body fluid of a subject.

Prognostic Uses of B7-H4 Antibodies.

The present disclosure also provides for prognostic (or predictive) assays for determining whether a subject is at risk of developing a medical disease or condition associated with increased B7-H4 polypeptide expression or activity (e.g., detection of a precancerous cell) or alternatively, to detect a tumor that may be suitable to treatment with a CAR T cell of this disclosure. Such assays can be used for prognostic or predictive purpose to thereby prophylactically treat an individual prior to the onset of a medical disease or condition characterized by or associated with B7-H4 polypeptide expression.

Another aspect of the present disclosure provides methods for determining B7-H4 expression in a subject to thereby select appropriate therapeutic or prophylactic compounds for that subject.

Alternatively, the prognostic assays can be utilized to identify a subject having or at risk for developing cancer and/or solid tumors. In certain embodiments, the cancer and/or tumor is of the breast, colon, prostate, ovary or more specifically a chrio-carcinoma. Thus, the present disclosure provides a method for identifying a disease or condition associated with increased B7-H4 polypeptide expression levels in which a test sample is obtained from a subject and the B7-H4 polypeptide detected, wherein the presence of increased levels of B7-H4 polypeptides compared to a control sample is predictive for a subject having or at risk of developing a disease or condition associated with increased B7-H4 polypeptide expression levels. In some aspects, the disease or condition associated with increased B7-H4 polypeptide expression levels is selected from the group consisting of cancer and/or solid tumors. In certain embodiments, the cancer and/or tumor is of the breast, colon, prostate, ovary or more specifically a chrio-carcinoma.

In another aspect, the present disclosure provides methods for determining whether a subject can be effectively treated with a compound for a disorder or condition associated with increased B7-H4 polypeptide expression wherein a biological sample is obtained from the subject and the B7-H4 polypeptide is detected using the B7-H4 antibody. The expression level of the B7-H4 polypeptide in the biological sample obtained from the subject is determined and compared with the B7-H4 expression levels found in a biological sample obtained from a subject who is free of the disease. Elevated levels of the B7-H4 polypeptide in the sample obtained from the subject suspected of having the disease or condition compared with the sample obtained from the healthy subject is indicative of the B7-H4-associated disease or condition in the subject being tested.

There are a number of disease states in which the elevated expression level of B7-H4 polypeptides is known to be indicative of whether a subject with the disease is likely to respond to a particular type of therapy or treatment. Thus, the method of detecting a B7-H4 polypeptide in a biological sample can be used as a method of prognosis, e.g., to evaluate the likelihood that the subject will respond to the therapy or treatment. The level of the B7-H4 polypeptide in a suitable tissue or body fluid sample from the subject is determined and compared with a suitable control, e.g., the level in subjects with the same disease but who have responded favorably to the treatment.

In one aspect, the present disclosure provides for methods of monitoring the influence of agents (e.g., drugs, compounds, or small molecules) on the expression of B7-H4 polypeptides. Such assays can be applied in basic drug screening and in clinical trials. For example, the effectiveness of an agent to decrease B7-H4 polypeptide levels can be monitored in clinical trials of subjects exhibiting elevated expression of B7-H4, e.g., patients diagnosed with cancer. An agent that affects the expression of B7-H4 polypeptides can be identified by administering the agent and observing a response. In this way, the expression pattern of the B7-H4 polypeptide can serve as a marker, indicative of the physiological response of the subject to the agent. Accordingly, this response state may be determined before, and at various points during, treatment of the subject with the agent.

Further method aspects of the present disclosure relate to methods for determining if a patient is likely to respond or is not likely to B7-H4 CAR therapy. In specific embodiments, this method comprises contacting a tumor sample isolated from the patient with an effective amount of an B7-H4 antibody and detecting the presence of any antibody bound to the tumor sample. In further embodiments, the presence of antibody bound to the tumor sample indicates that the patient is likely to respond to the B7-H4 CAR therapy and the absence of antibody bound to the tumor sample indicates that the patient is not likely to respond to the B7-H4 therapy. In some embodiments, the method comprises the additional step of administering an effective amount of the B7-H4 CAR therapy to a patient that is determined likely to respond to the B7-H4 CAR therapy. In some embodiments, the patient a B7-H4 expressing tumor and/or cancer. In some embodiments, the tumor and/or cancer is a solid tumor, e.g., breast, colon or chorio-carcinoma.

IV. Kits

As set forth herein, the present disclosure provides diagnostic methods for determining the expression level of B7-H4. In one particular aspect, the present disclosure provides kits for performing these methods as well as instructions for carrying out the methods of the present disclosure such as collecting tissue and/or performing the screen, and/or analyzing the results.

The kit comprises, or alternatively consists essentially of, or yet further consists of, a B7-H4 antibody composition (e.g., monoclonal antibodies) disclosed herein, and instructions for use. The kits are useful for detecting the presence of B7-H4 polypeptides in a biological sample e.g., any body fluid including, but not limited to, e.g., sputum, serum, plasma, lymph, cystic fluid, urine, stool, cerebrospinal fluid, acitic fluid or blood and including biopsy samples of body tissue. The test samples may also be a tumor cell, a normal cell adjacent to a tumor, a normal cell corresponding to the tumor tissue type, a blood cell, a peripheral blood lymphocyte, or combinations thereof. The test sample used in the above-described method will vary based on the assay format, nature of the detection method and the tissues, cells or extracts used as the sample to be assayed. Methods for preparing protein extracts or membrane extracts of cells are known in the art and can be readily adapted in order to obtain a sample which is compatible with the system utilized.

In some aspects, the kit can comprise: one or more B7-H4 antibodies capable of binding a B7-H4 polypeptide in a biological sample (e.g., an antibody or antigen-binding fragment thereof having the same antigen-binding specificity of B7-H4 antibody B7H4 5F6, B7H4 #33-14, or B7H4 #36-1); means for determining the amount of the B7-H4 polypeptide in the sample; and means for comparing the amount of the B7-H4 polypeptide in the sample with a standard. One or more of the B7-H4 antibodies may be labeled. The kit components, (e.g., reagents) can be packaged in a suitable container. The kit can further comprise instructions for using the kit to detect the B7-H4 polypeptides. In certain aspects, the kit comprises a first antibody, e.g., attached to a solid support, which binds to a B7-H4 polypeptide; and, optionally; 2) a second, different antibody which binds to either the B7-H4 polypeptide or the first antibody and is conjugated to a detectable label.

The kit can also comprise, e.g., a buffering agent, a preservative or a protein-stabilizing agent. The kit can further comprise components necessary for detecting the detectable-label, e.g., an enzyme or a substrate. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present disclosure may contain a written product on or in the kit container. The written product describes how to use the reagents contained in the kit.

As amenable, these suggested kit components may be packaged in a manner customary for use by those of skill in the art. For example, these suggested kit components may be provided in solution or as a liquid dispersion or the like.

V. Carriers

The antibodies also can be bound to many different carriers. Thus, this disclosure also provides compositions containing the antibodies and another substance, active or inert. Examples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the disclosure. Those skilled in the art will know of other suitable carriers for binding antibodies, or will be able to ascertain such, using routine experimentation.

### Chimeric Antigen Receptors and Uses Thereof

I. Compositions

The present disclosure provides chimeric antigen receptors (CAR) that bind to B7-H4 comprising, or consisting essentially of, a cell activation moiety comprising an extracellular, transmembrane, and intracellular domain. The extracellular domain comprises a target-specific binding element otherwise referred to as the antigen binding domain. The intracellular domain or cytoplasmic domain comprises, a costimulatory signaling region and a zeta chain portion. The CAR may optionally further comprise a spacer domain of up to 300 amino acids, preferably 10 to 100 amino acids, more preferably 25 to 50 amino acids.

Antigen Binding Domain.

In certain aspects, the present disclosure provides a CAR that comprises, or alternatively consists essentially thereof, or yet consists of an antigen binding domain specific to B7-H4. In some embodiments, the antigen binding domain comprises, or alternatively consists essentially thereof, or yet consists of the antigen binding domain of an anti-B7-H4 antibody. In further embodiments, the heavy chain variable region and light chain variable region of an anti-B7-H4 antibody comprises, or alternatively consists essentially thereof, or yet consists of the antigen binding domain the anti-B7-H4 antibody.

In some embodiments, the heavy chain variable region of the antibody comprises, or consists essentially thereof, or consists of SEQ ID NOs: 12 to 17 or an equivalent thereof and/or comprises one or more CDR regions comprising SEQ ID NOs: 1 to 11 or an equivalent thereof. In some embodiments, the light chain variable region of the antibody comprises, or consists essentially thereof, or consists of SEQ ID NOs: 28 to 33 or an equivalent thereof and/or comprises one or more CDR regions comprising SEQ ID NOs: 18 to 27 or an equivalent thereof.

Transmembrane Domain.

The transmembrane domain may be derived either from a natural or from a synthetic source. Where the source is natural, the domain may be derived from any membrane-bound or transmembrane protein. Transmembrane regions of particular use in this disclosure may be derived from CD8, CD28, CD3, CD45, CD4, CD5, CD5, CD9, CD 16, CD22, CD33, CD37, CD64, CD80, CD86, CD 134, CD137, CD 154, TCR. Alternatively the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

Cytoplasmic Domain.

The cytoplasmic domain or intracellular signaling domain of the CAR is responsible for activation of at least one of the traditional effector functions of an immune cell in which a CAR has been placed. The intracellular signaling domain refers to a portion of a protein which transduces the effector function signal and directs the immune cell to perform its specific function. An entire signaling domain or a truncated portion thereof may be used so long as the truncated portion is sufficient to transduce the effector function signal. Cytoplasmic sequences of the TCR and co-receptors as well as derivatives or variants thereof can function as intracellular signaling domains for use in a CAR. Intracellular signaling domains of particular use in this disclosure may be derived from FcR, TCR, CD3, CDS, CD22, CD79a, CD79b, CD66d. Since signals generated through the TCR are alone insufficient for full activation ofa T cell, a secondary or co-stimulatory signal may also be required. Thus, the intracellular region of a co-stimulatory signaling molecule, including but not limited CD27, CD28, 4-IBB (CD 137), OX40, CD30, CD40, PD-1, ICOS, lymphocyte function-associated antigen-1 (LFA-1), CD2, CD7, LIGHT, NKG2C, B7-H3, or a ligand that specifically binds with CD83, to may also be included in the cytoplasmic domain of the CAR.

In some embodiments, the cell activation moiety of the chimeric antigen receptor is a T-cell signaling domain comprising, or alternatively consisting essentially of, or yet further consisting of, one or more proteins or fragments thereof selected from the group consisting of CD8 protein, CD28 protein, 4-1BB protein, and CD3-zeta protein.

In specific embodiments, the CAR comprises, or alternatively consists essentially thereof, or yet consists of an antigen binding domain of an anti-B7-H4 antibody, a CD8 α hinge domain, a CD8 α transmembrane domain, a costimulatory signaling region, and a CD3 zeta signaling domain. In further embodiments, the costimulatory signaling region comprises either or both a CD28 costimulatory signaling region and a 4-1BB costimulatory signaling region.

In some embodiments, the CAR can further comprise a detectable marker or purification marker.

II. Process for Preparing CARs

Also provided herein is a method of producing B7-H4 CAR expressing cells comprising, or alternatively consisting essentially of, or yet further consisting of the steps: (i) transducing a population of isolated cells with a nucleic acid sequence encoding the CAR as described herein; and (ii) selecting a subpopulation of said isolated cells that have been successfully transduced with said nucleic acid sequence of step (i) thereby producing B7-H4 CAR expressing cells. In one aspect, the isolated cells are selected from a group consisting of T-cells and NK-cells.

Aspects of the present disclosure relate to an isolated cell comprising a B7-H4 CAR and methods of producing such cells. The cell is a prokaryotic or a eukaryotic cell. In one aspect, the cell is a T cell or an NK cell. The eukaryotic cell can be from any preferred species, e.g., an animal cell, a mammalian cell such as a human, a feline or a canine cell.

In specific embodiments, the isolated cell comprises, or alternatively consists essentially of, or yet further consists of an exogenous CAR comprising, or alternatively consisting essentially of, or yet further consisting of, an antigen binding domain of an anti-B7-H4 antibody, a CD8 α hinge domain, a CD8 α transmembrane domain, a CD28 costimulatory signaling region and/or a 4-1BB costimulatory signaling region, and a CD3 zeta signaling domain. In certain embodiments, the isolated cell is a T-cell, e.g., an animal T-cell, a mammalian T-cell, a feline T-cell, a canine T-cell or a human T-cell. In certain embodiments, the isolated cell is an NK-cell, e.g., an animal NK-cell, a mammalian NK-cell, a feline NK-cell, a canine NK-cell or a human NK-cell.

In certain embodiments, methods of producing B7-H4 CAR expressing cells are disclosed comprising, or alternatively consisting essentially of: (i) transducing a population of isolated cells with a nucleic acid sequence encoding a B7-H4 CAR and (ii) selecting a subpopulation of cells that have been successfully transduced with said nucleic acid sequence of step (i). In some embodiments, the isolated cells are T-cells, an animal T-cell, a mammalian T-cell, a feline T-cell, a canine T-cell or a human T-cell, thereby producing B7-H4 CAR T-cells. In certain embodiments, the isolated cell is an NK-cell, e.g., an animal NK-cell, a mammalian NK-cell, a feline NK-cell, a canine NK-cell or a human NK-cell, thereby producing B7-H4 CAR NK-cells.

Sources of Isolated Cells.

Prior to expansion and genetic modification of the cells disclosed herein, cells may be obtained from a subject—for instance, in embodiments involving autologous therapy—or a commercially available culture, that are available from the American Type Culture Collection (ATCC), for example.

Cells can be obtained from a number of sources in a subject, including peripheral blood mononuclear cells, bone marrow, lymph node tissue, cord blood, thymus tissue, tissue from a site of infection, ascites, pleural effusion, spleen tissue, and tumors.

Methods of isolating relevant cells are well known in the art and can be readily adapted to the present application; an exemplary method is described in the examples below. Isolation methods for use in relation to this disclosure include, but are not limited to Life Technologies Dynabeads® system; STEMcell Technologies EasySep™, RoboSep™, RosetteSep™, SepMate™; Miltenyi Biotec MACS™ cell separation kits, and other commercially available cell separation and isolation kits. Particular subpopulations of immune cells may be isolated through the use of beads or other binding agents available in such kits specific to unique cell surface markers. For example, MACS™ CD4+ and CD8+ MicroBeads may be used to isolate CD4+ and CD8+ T-cells

Alternatively, cells may be obtained through commercially available cell cultures, including but not limited to, for T-cells, lines BCL2 (AAA) Jurkat (ATCC® CRL-2902™), BCL2 (S70A) Jurkat (ATCC® CRL-2900™), BCL2 (S87A) Jurkat (ATCC® CRL-2901™), BCL2 Jurkat (ATCC® CRL-2899™), Neo Jurkat (ATCC® CRL-2898™); and, for NK cells, lines NK-92 (ATCC® CRL-2407™), NK-92MI (ATCC® CRL-2408™).

Vectors.

CARs may be prepared using vectors. Aspects of the present disclosure relate to an isolated nucleic acid sequence encoding a B7-H4 CAR and vectors comprising, or alternatively consisting essentially of, or yet further consisting of, an isolated nucleic acid sequence encoding the CAR and its complement and equivalents of each thereof.

In some embodiments, the isolated nucleic acid sequence encodes for a CAR comprising, or alternatively consisting essentially of, or yet further consisting of an antigen binding domain of an anti-B7-H4 antibody, a CD8 α hinge domain, a CD8 α transmembrane domain, a CD28 costimulatory signaling region and/or a 4-1BB costimulatory signaling region, and a CD3 zeta signaling domain. In specific embodiments, the isolated nucleic acid sequence comprises, or alternatively consisting essentially thereof, or yet further consisting of, sequences encoding (a) an antigen binding domain of an anti-B7-H4 antibody followed by (b) a CD8 α hinge domain, (c) a CD8 α transmembrane domain followed by (d) a CD28 costimulatory signaling region and/or a 4-1BB costimulatory signaling region followed by (e) a CD3 zeta signaling domain. The polypeptides encoded by these aspect are disclosed herein.

In some embodiments, the isolated nucleic acid sequence comprises, or alternatively consists essentially thereof, or yet further consists of, a Kozak consensus sequence upstream of the sequence encoding the antigen binding domain of the anti-B7-H4 antibody. In some embodiments, the isolated nucleic acid comprises a polynucleotide conferring antibiotic resistance.

In some embodiments, the isolated nucleic acid sequence is comprised in a vector. In certain embodiments, the vector is a plasmid. In other embodiments, the vector is a viral vector. In specific embodiments, the vector is a lentiviral vector.

The preparation of exemplary vectors and the generation of CAR expressing cells using said vectors is discussed in detail in the examples below. In summary, the expression of natural or synthetic nucleic acids encoding CARs is typically achieved by operably linking a nucleic acid encoding the CAR polypeptide or portions thereof to a promoter, and incorporating the construct into an expression vector. The vectors can be suitable for replication and integration eukaryotes. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York).

In one aspect, the term “vector” intends a recombinant vector that retains the ability to infect and transduce non-dividing and/or slowly-dividing cells and integrate into the target cell's genome. In several aspects, the vector is derived from or based on a wild-type virus. In further aspects, the vector is derived from or based on a wild-type lentivirus. Examples of such, include without limitation, human immunodeficiency virus (HIV), equine infectious anemia virus (EIAV), simian immunodeficiency virus (SIV) and feline immunodeficiency virus (FIV). Alternatively, it is contemplated that other retrovirus can be used as a basis for a vector backbone such murine leukemia virus (MLV). It will be evident that a viral vector according to the disclosure need not be confined to the components of a particular virus. The viral vector may comprise components derived from two or more different viruses, and may also comprise synthetic components. Vector components can be manipulated to obtain desired characteristics, such as target cell specificity.

The recombinant vectors of this disclosure are derived from primates and non-primates. Examples of primate lentiviruses include the human immunodeficiency virus (HIV), the causative agent of human acquired immunodeficiency syndrome (AIDS), and the simian immunodeficiency virus (SIV). The non-primate lentiviral group includes the prototype “slow virus” visna/maedi virus (VMV), as well as the related caprine arthritis-encephalitis virus (CAEV), equine infectious anemia virus (EIAV) and the more recently described feline immunodeficiency virus (FIV) and bovine immunodeficiency virus (BIV). Prior art recombinant lentiviral vectors are known in the art, e.g., see U.S. Pat. Nos. 6,924,123; 7,056,699, 7,07,993; 7,419,829 and 7,442,551, incorporated herein by reference.

U.S. Pat. No. 6,924,123 discloses that certain retroviral sequence facilitate integration into the target cell genome. This patent teaches that each retroviral genome comprises genes called gag, pol and env which code for virion proteins and enzymes. These genes are flanked at both ends by regions called long terminal repeats (LTRs). The LTRs are responsible for proviral integration, and transcription. They also serve as enhancer-promoter sequences. In other words, the LTRs can control the expression of the viral genes. Encapsidation of the retroviral RNAs occurs by virtue of a psi sequence located at the 5′ end of the viral genome. The LTRs themselves are identical sequences that can be divided into three elements, which are called U3, R and U5. U3 is derived from the sequence unique to the 3′ end of the RNA. R is derived from a sequence repeated at both ends of the RNA, and U5 is derived from the sequence unique to the 5′end of the RNA. The sizes of the three elements can vary considerably among different retroviruses. For the viral genome. and the site of poly (A) addition (termination) is at the boundary between R and U5 in the right hand side LTR. U3 contains most of the transcriptional control elements of the provirus, which include the promoter and multiple enhancer sequences responsive to cellular and in some cases, viral transcriptional activator proteins.

With regard to the structural genes gag, pol and env themselves, gag encodes the internal structural protein of the virus. Gag protein is proteolytically processed into the mature proteins MA (matrix), CA (capsid) and NC (nucleocapsid). The pol gene encodes the reverse transcriptase (RT), which contains DNA polymerase, associated RNase H and integrase (IN), which mediate replication of the genome.

For the production of viral vector particles, the vector RNA genome is expressed from a DNA construct encoding it, in a host cell. The components of the particles not encoded by the vector genome are provided in trans by additional nucleic acid sequences (the “packaging system”, which usually includes either or both of the gag/pol and env genes) expressed in the host cell. The set of sequences required for the production of the viral vector particles may be introduced into the host cell by transient transfection, or they may be integrated into the host cell genome, or they may be provided in a mixture of ways. The techniques involved are known to those skilled in the art.

Retroviral vectors for use in this disclosure include, but are not limited to Invitrogen's pLenti series versions 4, 6, and 6.2 “ViraPower” system. Manufactured by Lentigen Corp.; pHIV-7-GFP, lab generated and used by the City of Hope Research Institute; “Lenti-X” lentiviral vector, pLVX, manufactured by Clontech; pLKO.1-puro, manufactured by Sigma-Aldrich; pLemiR, manufactured by Open Biosystems; and pLV, lab generated and used by Charité Medical School, Institute of Virology (CBF), Berlin, Germany.

Regardless of the method used to introduce exogenous nucleic acids into a host cell or otherwise expose a cell to the inhibitor of the present disclosure, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the disclosure.

Packaging Vector and Cell Lines.

CARs can be packaged into a retroviral packaging system by using a packaging vector and cell lines. The packaging plasmid includes, but is not limited to retroviral vector, lentiviral vector, adenoviral vector, and adeno-associated viral vector. The packaging vector contains elements and sequences that facilitate the delivery of genetic materials into cells. For example, the retroviral constructs are packaging plasmids comprising at least one retroviral helper DNA sequence derived from a replication-incompetent retroviral genome encoding in trans all virion proteins required to package a replication incompetent retroviral vector, and for producing virion proteins capable of packaging the replication-incompetent retroviral vector at high titer, without the production of replication-competent helper virus. The retroviral DNA sequence lacks the region encoding the native enhancer and/or promoter of the viral 5′ LTR of the virus, and lacks both the psi function sequence responsible for packaging helper genome and the 3′ LTR, but encodes a foreign polyadenylation site, for example the SV40 polyadenylation site, and a foreign enhancer and/or promoter which directs efficient transcription in a cell type where virus production is desired. The retrovirus is a leukemia virus such as a Moloney Murine Leukemia Virus (MMLV), the Human Immunodeficiency Virus (HIV), or the Gibbon Ape Leukemia virus (GALV). The foreign enhancer and promoter may be the human cytomegalovirus (HCMV) immediate early (IE) enhancer and promoter, the enhancer and promoter (U3 region) of the Moloney Murine Sarcoma Virus (MMSV), the U3 region of Rous Sarcoma Virus (RSV), the U3 region of Spleen Focus Forming Virus (SFFV), or the HCMV IE enhancer joined to the native Moloney Murine Leukemia Virus (MMLV) promoter. The retroviral packaging plasmid may consist of two retroviral helper DNA sequences encoded byplasmid based expression vectors, for example where a first helper sequence contains a cDNA encoding the gag and pol proteins of ecotropic MMLV or GALV and a second helper sequence contains a cDNA encoding the env protein. The Env gene, which determines the host range, may be derived from the genes encoding xenotropic, amphotropic, ecotropic, polytropic (mink focus forming) or 10A1 murine leukemia virus env proteins, or the Gibbon Ape Leukemia Virus (GALV env protein, the Human Immunodeficiency Virus env (gp160) protein, the Vesicular Stomatitus Virus (VSV) G protein, the Human T cell leukemia (HTLV) type I and II env gene products, chimeric envelope gene derived from combinations of one or more of the aforementioned env genes or chimeric envelope genes encoding the cytoplasmic and transmembrane of the aforementioned env gene products and a monoclonal antibody directed against a specific surface molecule on a desired target cell.

In the packaging process, the packaging plasmids and retroviral vectors expressing the B7-H4 are transiently cotransfected into a first population of mammalian cells that are capable of producing virus, such as human embryonic kidney cells, for example 293 cells (ATCC No. CRL1573, ATCC, Rockville, Md.) to produce high titer recombinant retrovirus-containing supernatants. In another method of the invention this transiently transfected first population of cells is then cocultivated with mammalian target cells, for example human lymphocytes, to transduce the target cells with the foreign gene at high efficiencies. In yet another method of the invention the supernatants from the above described transiently transfected first population of cells are incubated with mammalian target cells, for example human lymphocytes or hematopoietic stem cells, to transduce the target cells with the foreign gene at high efficiencies.

In another aspect, the packaging plasmids are stably expressed in a first population of mammalian cells that are capable of producing virus, such as human embryonic kidney cells, for example 293 cells. Retroviral or lentiviral vectors are introduced into cells by either cotransfection with a selectable marker or infection with pseudotyped virus. In both cases, the vectors integrate. Alternatively, vectors can be introduced in an episomally maintained plasmid. High titer recombinant retrovirus-containing supernatants are produced.

Activation and Expansion of T Cells.

Whether prior to or after genetic modification of the T cells to express a desirable CAR, the cells can be activated and expanded using generally known methods such as those described in U.S. Pat. Nos. 6,352,694; 6,534,055; 6,905,680; 6,692,964; 5,858,358; 6,887,466; 6,905,681; 7, 144,575; 7,067,318; 7, 172,869; 7,232,566; 7, 175,843; 5,883,223; 6,905,874; 6,797,514; 6,867,041. Stimulation with the B7-H4 antigen ex vivo can activate and expand the selected CAR expressing cell subpopulation. Alternatively, the cells may be activated in vivo by interaction with B7-H4 antigen.

Methods of activating relevant cells are well known in the art and can be readily adapted to the present application; an exemplary method is described in the examples below. Isolation methods for use in relation to this disclosure include, but are not limited to Life Technologies Dynabeads® system activation and expansion kits; BD Biosciences Phosflow™ activation kits, Miltenyi Biotec MACS™ activation/expansion kits, and other commercially available cell kits specific to activation moieties of the relevant cell. Particular subpopulations of immune cells may be activated or expanded through the use of beads or other agents available in such kits. For example, α-CD3/α-CD28 Dynabeads® may be used to activate and expand a population of isolated T-cells.

III. Methods of Use

Therapeutic Application.

Method aspects of the present disclosure relate to methods for inhibiting the growth of a tumor in a subject in need thereof and/or for treating a cancer patient in need thereof. In some embodiments, the tumors/cancer is a solid tumor, e.g., breast, colon, chrio-carcinoma, ovarian or prostate. In some embodiments, the tumor or cancer expresses or overexpresses B7-H4. In certain embodiments, these methods comprise, or alternatively consist essentially of, or yet further consist of, administering to the subject or patient an effective amount of an isolated cell. In further embodiments, this isolated cell comprises a B7-H4 CAR. In still further embodiments, the isolated cell is a T-cell or an NK cell. In some embodiments, the isolated cell is autologous to the subject or patient being treated. In a further aspect, the tumor expresses B7-H4 antigen and the subject has been selected for the therapy by a diagnostic, such as the one described herein.

The CAR cells as disclosed herein may be administered either alone or in combination with diluents, known anti-cancer therapeutics, and/or with other components such as cytokines or other cell populations that are immunostimulatory. They may be administered as a first line therapy, a second line therapy, a third line therapy, or further therapy. Non-limiting examples of additional therapies include chemotherapeutics or biologics. Appropriate treatment regimens will be determined by the treating physician or veterinarian.

Pharmaceutical compositions of the present invention may be administered in a manner appropriate to the disease to be treated or prevented. The quantity and frequency of administration will be determined by such factors as the condition of the patient, and the type and severity of the patient's disease, although appropriate dosages may be determined by clinical trials.

### Carriers

Additional aspects of the invention relate to compositions comprising a carrier and one or more of the products—e.g., an isolated cell comprising a B7-H4 CAR, an isolated nucleic acid, a vector, an isolated cell of any anti-B7-H4 antibody or CAR cell, an anti-B7-H4—described in the embodiments disclosed herein.

Briefly, pharmaceutical compositions of the present invention including but not limited to any one of the claimed compositions may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure may be formulated for oral, intravenous, topical, enteral, and/or parenteral administration. In certain embodiments, the compositions of the present disclosure are formulated for intravenous administration.

Administration of the cells or compositions can be effected in one dose, continuously or intermittently throughout the course of treatment. Methods of determining the most effective means and dosage of administration are known to those of skill in the art and will vary with the composition used for therapy, the purpose of the therapy and the subject being treated. Single or multiple administrations can be carried out with the dose level and pattern being selected by the treating physician. Suitable dosage formulations and methods of administering the agents are known in the art. In a further aspect, the cells and composition of the invention can be administered in combination with other treatments.

The cells and populations of cell are administered to the host using methods known in the art and described, for example, in PCT/US2011/064191. This administration of the cells or compositions of the invention can be done to generate an animal model of the desired disease, disorder, or condition for experimental and screening assays.

The following examples are illustrative of procedures which can be used in various instances in carrying the disclosure into effect.

### Example 1—Generation of Mouse Anti-Human B7-H4 Monoclonal Antibodies

### 1. Construction of the B7H4-Fc Fusion Protein

Expression vector encoding the human B7-H4 signal and extracellular domains fused to the Fc region of human IgG₁ were constructed as follows: cDNA encoding the signal and extracellular domains of human B7-H4 were generated by PCR amplification from full-length cDNA purchased from Open Biosystem (Lafayette, Colo.). The cDNA extends from the initiation Met in the signal sequence through Gly₂₃₆ of the total protein sequence. Primary PCR of B7-H4 was performed with the 5′ and 3′ primers 5′-TCG ATC AAG CTT GCC GCC ACC ATG GCT TCC CTG GGG CAG ATC-3′ AND 5′-TGT GTG AGT TTT GTC AGC CTT TGA CAG CTG-3′, respectively. The hinge-CH2-CH3 portion of human IgG1 was PCR amplified with 5′ primer 5′-CTA AAC TCA AAG GCT GAC AAA ACT CAC ACA TGC CCA-3′ and 3′ primer 5′-TGA TTA ATG ATC AAT GAA TTC TCA TTT ACC CGG AGA CAG GGA-3′. The gene encoding huB7-H4-Fc was produced by assembling with 5′primer of B7-H4 and 3′ primer of human Fc, respectively. The full sequence of the B7-H4-Fc used was as follows (Bold: B7-H4 (SEQ ID NO: 43); Non-bold: human Fc):

(SEQ ID NO: 44)
IGEDGILSCTFEPDIKLSDIVIQWLKEGVLGLVHEFKEGKDELSEQDEMF
RGRTAVFADQVIVGNASLRLKNVQLTDAGTYKCYIITSKGKGNANLEYKT
GAFSMPEVNVDYNASSETLRCEAPRWFPQPTVVWASQVDQGANFSEVSNT
SFELNSENVTMKVVSVLYNVTINNTYSCMIENDIAKATGDIKVTESEIKR
RSHLQLLNSKADKTHTCPPCPAPELLGGPSVFLFPPKPKDTLMISRTPEV
TCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDELT
KNQVSLTCLVKGFYPSDIAVEWESNGQPENNYKTTPPVLDSDGSFFLYSK
LTVDKSRWQQGNVFSCSVMHEALHNHYTQKSLSLSPGK

The B7H4-Fc fusion gene was then digested with Hind3 and EcoR and inserted into Hind3 and EcoRl sites of pN24 expression vector, resulting in the expression vector pN24/B7-H4-Fc.

### 2. Expression, Purification, and Characterization of B7-H4-Fc Antigen

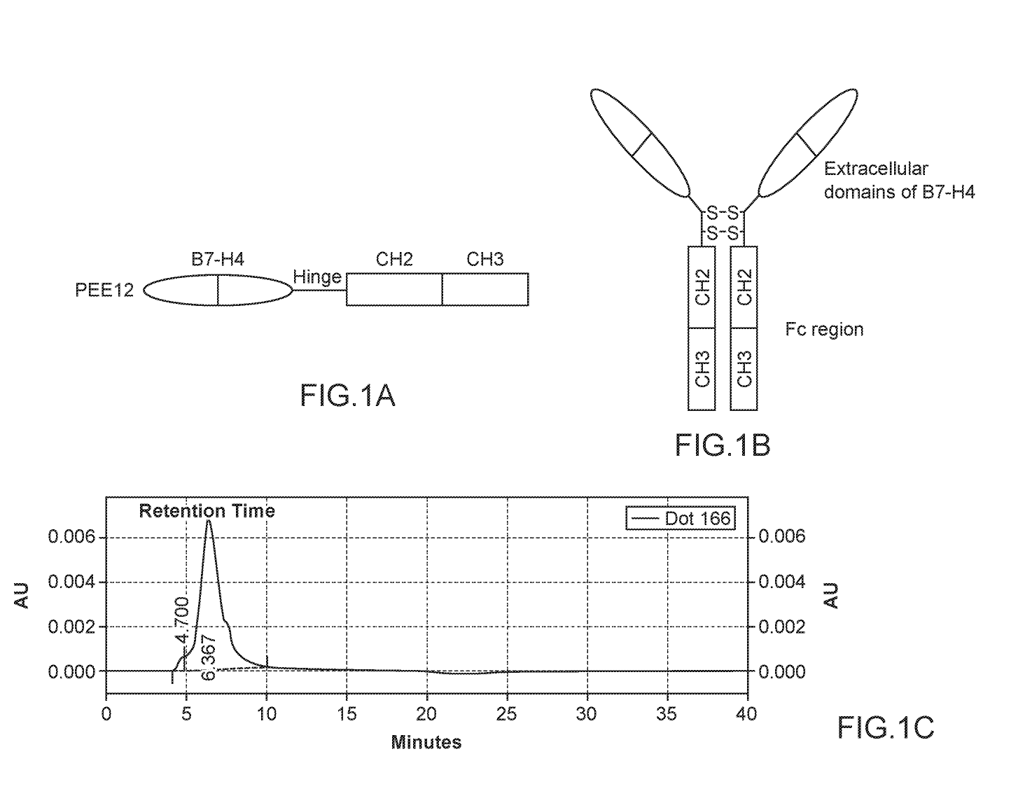

B7-H4-Fc fusion protein was expressed in NSO murine myeloma cells for long-term stable expression according to the manufacturer's protocol (Lonza Biologics, Inc.). The highest producing clone was scaled up for incubation in an aerated 3 L stir-flask bioreactor using 3% heat-inactivated dialyzed fetal calf serum. The fusion protein was then purified from the filtered spent culture medium by sequential Protein A affinity chromatography and ion-exchange chromatography procedures. The fusion protein was analyzed by HPLC and sodium dodecyl sulfate-polyacrylamide gel electrophoresis (SDS-PAGE) under reducing conditions and stained with Coomassie Blue to demonstrate proper assembly and purity. A schematic of the completed vector and molecule is shown in FIGS. 1A-1C along with HPLC data verifying its size.

### 3. Immunization Procedures

Four week old female BALB/c mice purchased from Harlan Laboratories were immunized every two weeks ×4 with 10 ug of KLH-conjugated huB7-H4-Fc emulsified with Complete Freund's Adjuvant (first and second immunization) or incomplete Freund's Adjuvant (third and fourth immunization). Mice were injected intradermally with a total of 25 ug of antigen/adjuvant divided into three separate spots on the back of the mice per immunization. Ten days after the last immunization, blood samples were obtained and tittered by ELISA procedures on antigen coated plates. Mice showing the highest titers then received a fifth immunization boost of B7-H4-Fc without adjuvant or KLH conjugation intravenously in which 10 ug were injected via the lateral tail vein in a 100 ul solution of sterile Phosphate Buffered Saline.

### 4. Hybridoma Production

Four days later, boosted mice were sacrificed and the spleens removed for the hybridoma procedure. After dispersing the splenocytes in a solution of RPMI-1640 medium containing Pen/Strep antibiotics, the splenocytes were fused with murine NSO cells using PEG (Hybri MAX, mol wt 1450, Cat. No: p7181, Sigma). HAT selection was then used to enable only fused cells to grow. Supernatant from wells with growing hybridoma cells were then screened initially by ELISA against B7-H4-Fc antigen coated plates and secondarily by flow cytometry on B7-H4 positive and negative human tumor cell lines (SK-BR-3 and HT-29, respectively). To eliminate positive hybridomas to the Fc region of B7-H4-Fc, supernatants were also screened against IL-2-Fc coated plates and those clones showing positivity to both antigens, were eliminated from further study. Hybridomas showing a positive and high mean fluorescent index (MFI) were selected for subcloning by limiting dilution methods. Subclones were then retested by flow cytometry, frozen in liquid nitrogen, and expanded in 2 L vessels before antibody was purified by tandori Protein A or G and ion exchange chromatography methods. Purified antibodies were then vialed and stored at −20° C. until used.

### 5. Flow Cytometry Data

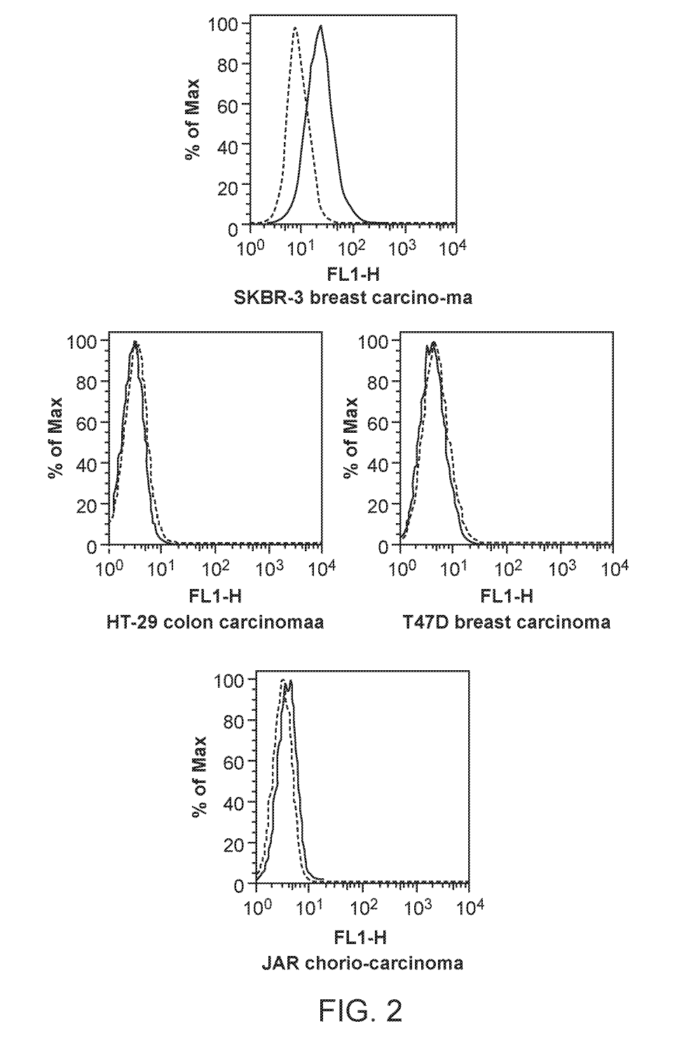

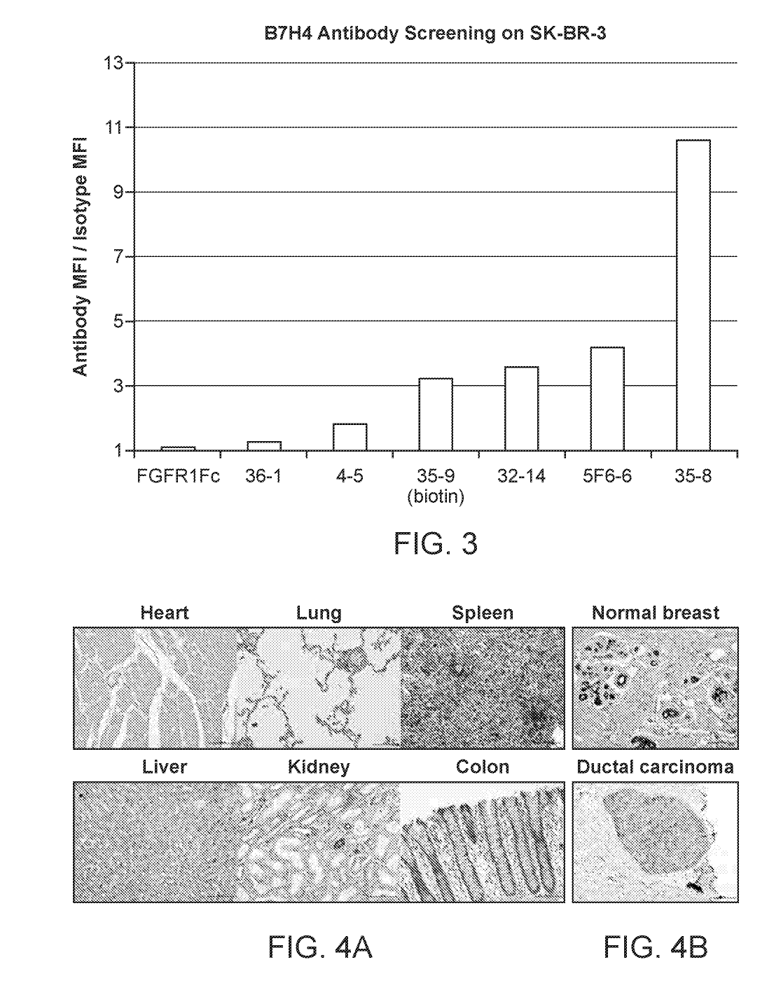

To determine the best binding antibodies, flow cytometry was performed on B7-H4 positive (SK-BR-3) and negative (HT-29, JAR, and T47D) cell lines using aliquots of purified antibodies. As shown in FIG. 2, positive cell lines had increased binding characteristics compared to negative antibody isotype controls. A comparison of positive subclones showed that hybridomas 35-8 and 5F6-6 produced the highest MFI to the B7-H4 expressing SK-BR-3 cell line (FIG. 3) and were therefore selected as candidates for CAR T-cell construction as described below.

### 6. Immunohistochemistry Data

Using these monoclonal antibodies, tissue microarrays (FDA808c, Biomax, Inc.) of human normal tissues were screened to determine antibody binding in 24 organs, with 3 donors per organ. While most tissues were negative for staining, there was inconsistent cytoplasmic staining in epithelial cells of the gastrointestinal tract, and in the proximal and distal convoluted tubules of the kidneys (FIGS. 4A-4B). Strong, consistent membranous staining was only found in the apical portion of breast ductal cells and in some of the tubules in the kidney (FIGS. 4A-4B). Staining in normal breast tissue, however, paled in comparison to staining in breast cancer tissue as shown below, where strong membranous and cytoplasmic staining was noted in five out of five different cancer cases.

From the antibodies generated against human B7-H4-Fc, two monoclonal antibodies have been shown to produce high binding profiles by flow cytometry against B7-H4 positive but not negative tumor cells lines. To prevent the possibility of a human anti-mouse response against B7-H4 CAR T-cell, humanized antibodies can be generated for their construction prior to their use in patients.

### Example 2—Generation of B7-H4 CAR T-Cells

### 1. Construction and Synthesis of Single Chain Anti-Human B7-H4 Antibody Genes

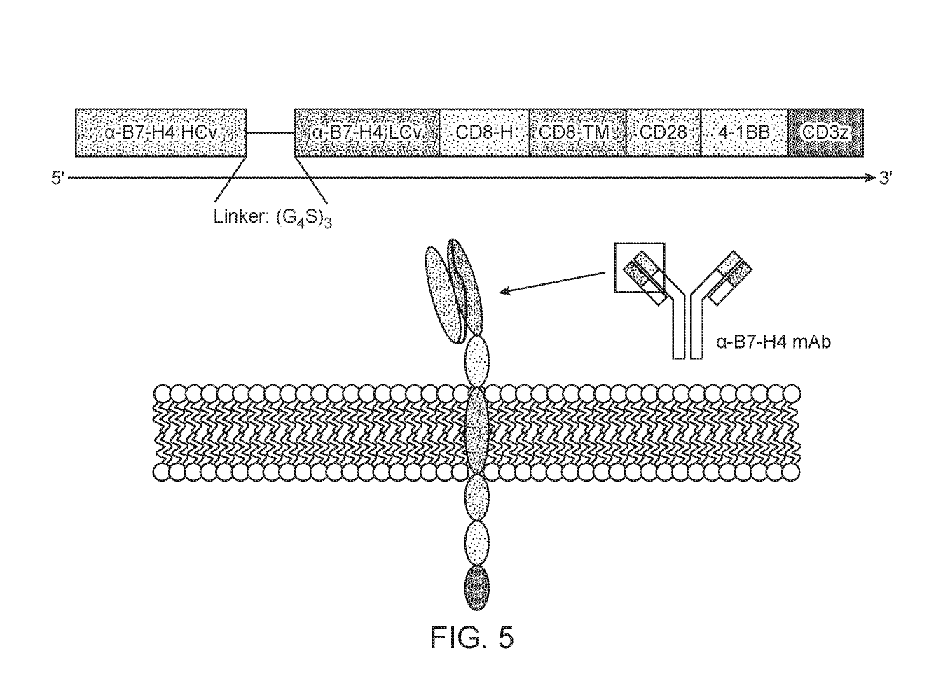

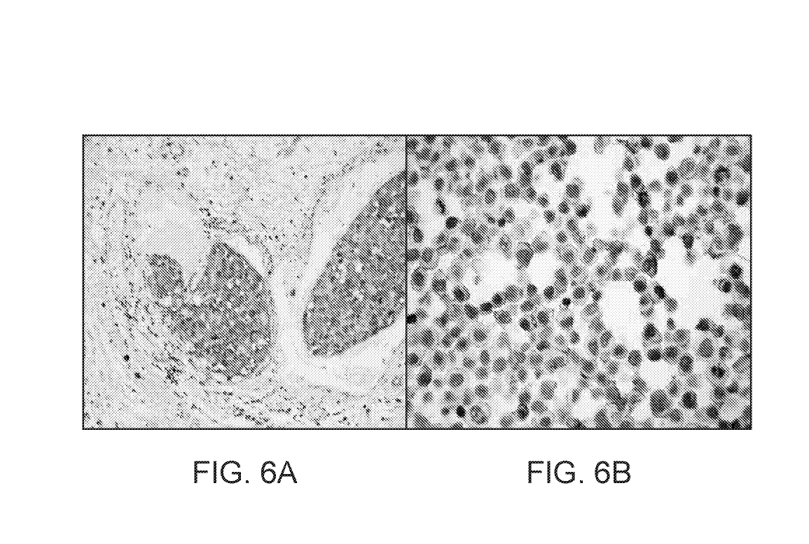

The DNA sequences for 35-8 and 5F6-6 high binding anti-B7-H4 antibodies generated are obtained from MCLAB (South San Francisco, Calif.). Both antibodies are tested to determine which one produces the most effective CAR T-cells in assays described below. For these studies, second or third (FIG. 5) generation CAR vectors are constructed consisting of the following tandem genes: a kozak consensus sequence; the CD8 signal peptide; the anti-B7-H4 heavy chain variable region; a (Glycine4Serine)3 flexible polypeptide linker: the respective anti-B7-H4 light chain variable region; CD8 hinge and transmembrane domains; and the CD28, 4-1BB, and CD3ζ intracellular co-stimulatory signaling domains. Hinge, transmembrane, and signaling domain DNA sequences are ascertained from a patent by Carl June (see US 20130287748 A1). Anti-B7-H4 CAR genes are synthesized by Genewiz, Inc. (South Plainfield, N.J.) within a pUC57 vector backbone containing the bla gene, which confers ampicillin resistance to the vector host.

2. Subcloning of CAR Genes into Lentiviral Plasmids

NovaBlue Singles™ chemically-competent E. coli cells are transformed with anti-B7-H4 plasmid cDNA. Following growth of the transformed E. coli cells, the CAR plasmids are purified and digested with the appropriate restriction enzymes to be inserted into an HIV-1-based lentiviral vector containing HIV-1 long terminal repeats (LTRs), packaging signal (Ψ), EF1α promoter, internal ribosome entry site (IRES), and woodchuck hepatitis virus post-transcriptional regulatory element (WPRE) via overnight T4 DNA ligase reaction (New England Biosciences; Ipswich, Mass.). NovaBlue Singles™ chemically-competent E. coli cells are then transformed with the resulting anti-B7-H4 containing lentiviral plasmid.

### 3. Production of Lentiviral Particles

Prior to transfection, HEK293T cells are seeded at 4.0×10⁶ cells/100 mm tissue-culture-treated plate in 10 mL complete-Tet-DMEM and incubated overnight at 37° C. in a humidified 5% CO₂ incubator. Once 80-90% confluent, HEK293T cells are co-transfected with CAR-gene lentiviral plasmids and lentiviral packaging plasmids containing genes necessary to form lentiviral envelope & capsid components, in addition to a proprietary reaction buffer and polymer to facilitate the formation of plasmid-containing nanoparticles that bind HEK293T cells. After incubating transfected-HEK293T cell cultures for 4 hours at 37° C., the transfection medium is replaced with 10 mL fresh complete Tet DMEM. HEK293T cells are then incubated for an additional 48 hours, after which cell supernatants are be harvested and tested for lentiviral particles via sandwich ELISA against p24, the main lentiviral capsid protein. Lentivirus-containing supernatants are aliquoted and stored at −80° C. until use for transduction of target CD4⁺ and CD8⁺ T cells.

4. Purification, Activation, and Enrichment of Human CD4⁺ and CD8⁺ Peripheral Blood T-Cells

Peripheral blood mononuclear cells (PBMCs) enriched by density gradient centrifugation with Ficoll-Paque Plus (GE Healthcare; Little Chalfont, Buckinghamshire, UK) are recovered and washed by centrifugation with PBS containing 0.5% bovine serum albumin (BSA) and 2 mM EDTA. MACS CD4⁺ and CD8⁺ MicroBeads (Miltenyi Biotec; San Diego, Calif.) kits are used to isolate these human T-cell subsets using magnetically activated LS columns to positive select for CD4⁺ and CD8⁺ T-cells. Magnetically-bound T-cells are then removed from the magnetic MACS separator, flushed from the LS column, and washed in fresh complete medium. The purity of CD4+ and CD8⁺ T-cell populations are assessed by flow cytometry using Life Technologies Acoustic Attune® Cytometer, and are enriched by Fluorescence-Activated Cell Sorting performed at USC's flow cytometry core facilities if needed. CD4⁺ and CD8⁺ T-cells are maintained at a density of 1.0×10⁶ cells/mL in complete medium supplemented with 100 IU/mL IL-2 in a suitable cell culture vessel, to which α-CD3/α-CD28 Human T-cell Dynabeads (Life Technologies; Carlsbad, Calif.) are added to activate cultured T cells. T-cells are incubated at 37° C. in a 5% CO₂ incubator for 2 days prior to transduction with CAR-lentiviral particles.

### 5. Lentiviral Transduction of CD4+ CD8+ T-Cells

Activated T-cells are collected and dead cells are removed by Ficoll-Hypaque density gradient centrifugation or the use of MACS Dead Cell Removal Kit (Miltenyi Biotec; San Diego, Calif.). In a 6-well plate, activated T-cells are plated at a concentration of 1.0×10⁶ cells/mL complete medium. To various wells, B7-H4 CAR-containing lentiviral particles are added to cell suspensions at varying multiplicity of infections (MOIs), such as 1, 5, 10, and 50. Polybrene, a cationic polymer that aids transduction by facilitating interaction between lentiviral particles and the target cell surface, is added at a final concentration of 4 g/mL. Plates are centrifuged at 800×g for 1 hr at 32° C. Following centrifugation, lentivirus-containing medium is aspirated and cell pellets are resuspended in fresh complete medium with 100 IU/mL IL-2. Cells are placed in a 5% CO₂ humidified incubator at 37° C. overnight. Three days post-transduction, cells are pelleted and resuspended in fresh complete medium with IL-2 and 400 μg/mL Geneticin (G418 sulfate) (Life Technologies; Carlsbad, Calif.). B7-H4 CAR modified T-cells are assessed by flow cytometry and southern blot analysis to demonstrate successful transduction procedures. Prior to in vitro and in vivo assays, B7-H4 CAR T-cells are enriched by FACS and mixed 1:1 for the in vivo studies.

### 6. In Vitro Assessment of CAR Efficacy by Calcein-Release Cytotoxicity Assays

B7-H4 antigen positive and negative human cell lines are collected, washed, and resuspended in complete medium at a concentration of 1.0×10⁶ cells/mL. Calcein-acetoxymethyl (AM) are added to target cell samples at 15 μM, which are then incubated at 37° C. in a 5% CO₂ humidified incubator for 30 minutes. Dyed positive and negative target cells are washed twice and resuspended in complete medium by centrifugation and added to a 96-well plate at 1.0×10⁴ cells/well. B7-H4 CAR T-cells are added to the plate in complete medium at effector-to-target cell ratios of 50:1, 5:1, and 1:1. Dyed-target cells suspended in complete medium and complete medium with 2% triton X-100 serve as spontaneous and maximal release controls, respectively. The plates are centrifuged at 365×g and 20° C. for 2 minutes before being placed back in the incubator 3 hours. The plates are then centrifuged 10 minutes and cell supernatants are aliquoted to respective wells on a black polystyrene 96-well plate and assessed for fluorescence on a Bio-Tek® Synergy™ HT microplate reader at excitation and emissions of 485/20 nm and 528/20 nm, respectively.

### 7. Quantification of Human Cytokines by Luminex Bioassay.

Supernatants of B7-H4 CAR modified T-cells and B7-H4 positive and negative tumor cell lines are measured for cytokine secretion as a measure of CAR T-cell activation using standard procedures performed routinely in the laboratory. Data are compared to medium alone and to cultures using non-activated human T-cells to identify background activity. The concentration of IL-2, IFN-g, IL-12, and other pertinent cytokines are measured over time during the incubation process.

### 8. In Vivo Assessment of CAR T-Cell Efficacy in Two Xenograft B7-H4 Positive Cancer Models

B7-H4 CAR T-cells are further evaluated in vivo using two different human tumor cell line xenograft tumor models. For both, solid tumors are established subcutaneously in 6-8 week old female nude mice by injection of 5×106 B7-H4 positive or negative solid tumor cell lines. When the tumors reach 0.5 cm in diameter, groups of mice (n=5) are treated intravenously with 1 or 3×10⁷ human T-cells as negative controls or B7-H4 CAR T-cells constructed from the candidate B7-H4 antibodies based upon the in vitro study results. Tumor volumes are then measured by caliper 3×/week and volume growth curves are generated to demonstrate the effectiveness of experimental treatments over controls.

In general, B7-H4 is expressed on tumors to suppress the immune response. Its expression on normal tissues is very limited making it a viable target for CAR T-cells.

### Example 3—Anti-B7-H4 CAR T-Cells

### Construction of the CAR Lentiviral Constructs

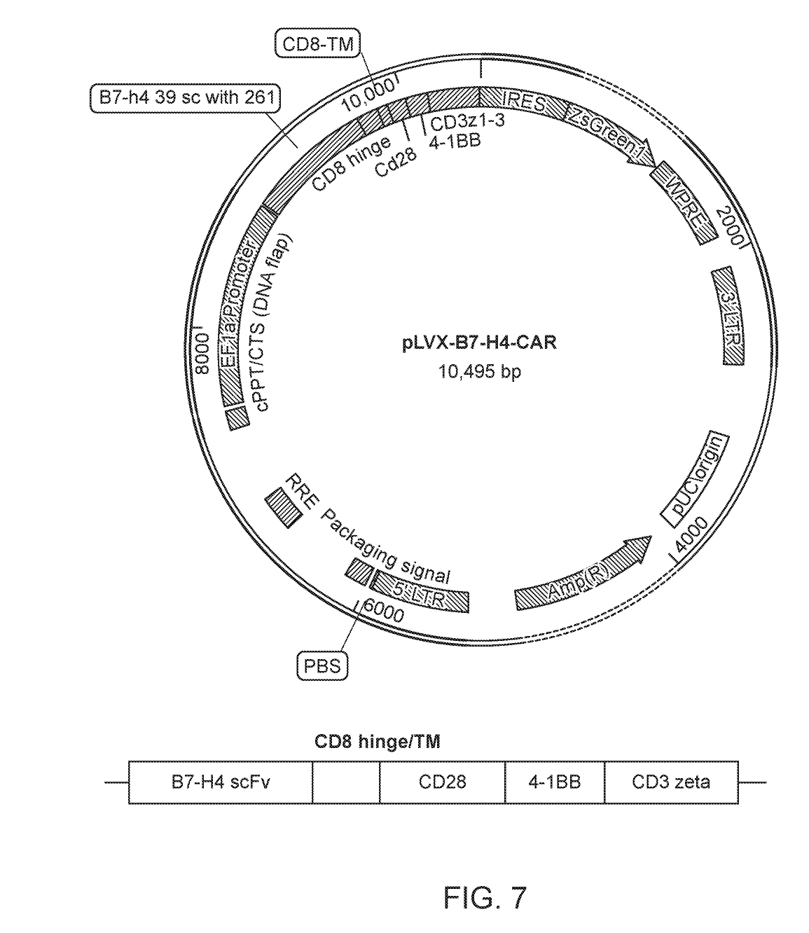

The CAR consists of an extracellular antigen binding moiety or scFV which binds B7-H4. The scFV is connected via a CD8 hinge region to the cytoplasmic signaling domain, comprised of the CD8 transmembrane region, and the signaling domains from CD28, 4-1 BB and CD3z (FIG. 7). The scFV sequence including the signaling domains, were synthetically synthesized by Genewiz Gene Synthesis services (Piscataway, N.J.). The plasmids are purified and digested with the appropriate restriction enzymes to be inserted into an HIV-1-based bicistronic lentiviral vector (pLVX-IRES-ZsGreen, Clontech, Signal Hill, Calif.) containing HIV-1 5′ and 3′ long terminal repeats (LTRs), packaging signal (PY), EF1α promoter, internal ribosome entry site (IRES), woodchuck hepatitis virus post-transcriptional regulatory element (WPRE) and simian virus 40 origin (SV40) via overnight T4 DNA ligase reaction (New England Biosciences; Ipswich, Mass.). NovaBlue Singles™ chemically-competent E. coli cells will then be transformed with the resulting CAR-containing lentiviral plasmid.

### Production of Lentiviral Particles

Prior to transfection, HEK 293T cells are seeded at 4.0×106 cells in a 150 cm2 tissue-culture-treated flask in 20 mL DMEM supplemented with 10% dialyzed FCS and incubated overnight at 37° C. in a humidified 5% CO2 incubator. Once 80-90% confluent, HEK 293T cells are incubated in 20 mL DMEM supplemented with 1-% dialyzed FCS without penicillin/streptamycin for two hours in at 37° C. in a humidified 5% CO2 incubator. HEK293T cells are co-transfected with the pLVX-B7-H4-CAR plasmid and lentiviral packaging plasmids containing genes necessary to form the lentiviral envelope & capsid components. A proprietary reaction buffer and polymer to facilitate the formation of plasmid-containing nanoparticles that bind HEK 293T cells are also added. After incubating the transfected-HEK 293T cell cultures for 24 hours at 37° C., the transfection medium is replaced with 20 mL fresh complete DMEM. Lentivirus supernatants is then collected every 24 hours for three days and the supernatants centrifuged at 1,250 rpm for 5 mins at 4° C., followed by filter sterilization and centrifugation in an ultracentrifuge at 20,000 g for 2 hrs at 4° C. The concentrated lentivirus is re-suspended in PBS containing 7% trehalose and 1% BSA. The lentivirus is then aliquoted and stored at −80° C. until use for transduction of target CD4+ and CD8+ T cells. The cell supernatants harvested after 24 hours are tested for lentiviral particles via sandwich ELISA against p24, the main lentiviral capsid protein. Transfection efficiency as determined by the expression of the protein marker ZsGreen was estimated between 20%-50%, by visualization under a fluorescent microscope.

Purification, Activation, and Enrichment of Human CD4⁺ and CD8⁺ Peripheral Blood T-Cells

Peripheral blood mononuclear cells (PBMCs) enriched by density gradient centrifugation with Ficoll-Paque Plus (GE Healthcare; Little Chalfont, Buckinghamshire, UK) are recovered and washed by centrifugation with PBS containing 0.5% bovine serum albumin (BSA) and 2 mM EDTA. T-cell enrichment kits (Stem Cell Technologies) are used to isolate these human T-cell subsets magnetically using negative selection for CD4+ and CD8+ T-cells. The purity of CD4+ and CD8+ T-cell populations are assessed by flow cytometry using Life Technologies Acoustic Attune® Cytometer, and are enriched by Fluorescence-Activated Cell Sorting. CD4+ and CD8+ T-cells mixed 1:1 are maintained at a density of 1.0×106 cells/mL in complete 50% Click's medium/50% RPMI-1640 medium supplemented with 100 IU/mL IL-2 in a suitable cell culture vessel, to which αCD3/αCD28 Human T-cell activator beads (Stem Cell Technologies) are added to activate the cultured T cells. T-cells are then incubated at 37° C. in a 5% C02 incubator for 2 days prior to transduction with CAR lentiviral particles.

Lentiviral Transduction of CD4⁺ CD8⁺ T-Cells

Activated T-cells are collected and dead cells are removed by Ficoll-Hypaque density gradient centrifugation or the use of MACS Dead Cell Removal Kit (Miltenyi Biotec; San Diego, Calif.). In a 6-well plate, activated T-cells are plated at a concentration of 1.0×106 cells/mL in complete medium. Cells are then transduced with the lentiviral particles supplemented with Lentiblast, a transfection aid (Oz Biosciences, San Diego, Calif.) to the cells. Transduced cells are incubated for 24 hours at 37° C. in a humidified 5% CO2 incubator. The cells are spun down and the media changed, followed by addition of the T-cell activator beads (Stem Cell Technologies, San Diego, Calif.).

### Cell Cytotoxicity Assays

Cytotoxicity of the CAR T-cells are determined using the lactate dehydrogenase (LDH) cytotoxicity kit (Thermo Scientific, Carlsbad, Calif.). Activated T-cells are collected and 1×10⁶ cells are transduced with the B7-H4 CAR lentiviral construct as described above. Cells are activated used the T-cell activator beads (Stem Cell Technologies, San Diego, Calif.) for two days prior to cytotoxicity assays. The optimal number of target cells is determined as per manufacturer's protocol. For the assays, the appropriate target cells are plated in triplicate in a 96 well plate for 24 hours at 37° C. in a 50% CO₂ incubator, followed by addition of activated CAR T-cells in ratios of 20:1, 10:1, 5:1 and 1:1, and incubated for 24 hours at 37° C. in a 5% CO₂ incubator. Cells are then lysed at 37° C. for 45 mins and centrifuged at 1,250 rpm for 5 mins. The supernatant is transferred to a fresh 96 well plate, followed by the addition of the reaction mixture for 30 mins. The reaction will be stopped using the stop solution and the plate read at 450 nm with an absorbance correction at 650 nm.

### In Vivo Tumor Regression Assay

Foxn1 null mice are injected with immortalized breast carcinoma cell line MDA-MB-468, which expresses B7-H4. Two×106 tumor cells in 200 ul of phosphate buffered saline (PBS) are injected into the left flank of the mice using a 0.2 mL inoculum. T-cells are activated for 2 days with the αCD3/CD28 activator complex (Stem Cell Technologies, San Diego, Calif.). The activated T-cells are then transduced with B7-H4 CAR lentiviral particles, followed by activation with the αCD3/CD28 activator complex for an additional 2 days. The activated B7-H4 CAR T-cells (2.5×106) are then injected intravenously into the mice on day 7 after tumor inoculation. Tumor sizes are assessed twice a week using Vernier calipers and the volume calculated.

### Cytotoxicity for B7-H4 CAR T-Cells

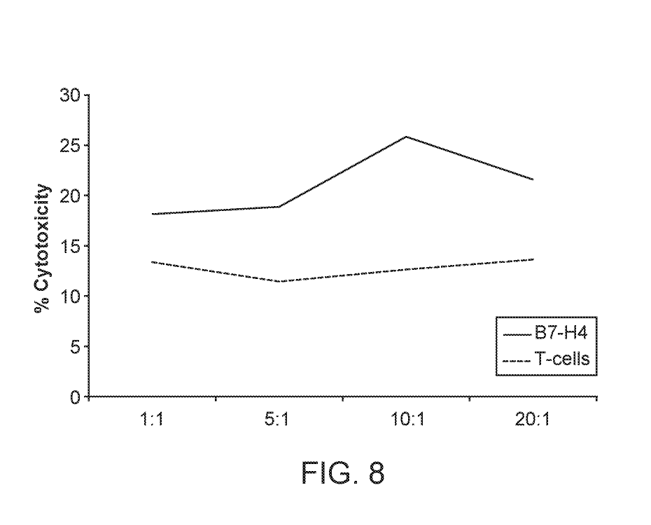

The cytolytic activity of the B7-H4 CAR T-cells was examined using SKBR3, a breast carcinoma cell line. SKBR3 expresses B7-H4, as determined by FACS analysis (FIG. 8). B7-H4 CAR T-cells were added to the SKBR3 in ratios of 20:1, 10:1, 5:1 and 1:1 of effector to target cells. At a ratio of 10,000:1, B7-H4 CAR T-cells show increased lysis of the target SKBR3 cells with a lysis rate of 25%. In comparison, untransduced T-cells did not lyse SKBR3 cells at any of the ratios tested.

### EQUIVALENTS

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

B7-H4 SEQUENCES
CDRH1
(SEQ ID NO: 1)
GXTF
(SEQ ID NO: 2)
GFTFSSFG
(SEQ ID NO: 3)
GFTFSSYG
(SEQ ID NO: 4)
GYTFTDY
CDRH2
(SEQ ID NO: 5)
ISSXXXT
(SEQ ID NO: 6)
ISSGSSTL
(SEQ ID NO: 7)
ISSSNSTI
(SEQ ID NO: 8)
INPNNGGT
CDRH3
(SEQ ID NO: 9)
ARPXYY
(SEQ ID NO: 10)
ARPLYYYGSVMDY
(SEQ ID NO: 11)
ARPYYYGSSYDY
HC1
(SEQ ID NO: 12)
GAGGTGCAGCTGGAGGAGTCTGGGGGAGGCTTAGTGCAGCCTGGAGGGTCCCGG
AAACTCTCCTGTGCAGCCTCTGGATTCACTTTCAGTAGCTTTGGAATGCACTGGG
TTCGTCAGGCTCCAGAGAAGGGGCTGGAGTGGGTCGCATACATTAGTAGTGGCA
GTAGTACCCTCCACTATGCAGACACAGTGAAGGGCCGATTCACCATCTCCAGAG
ACAATCCCAAGAACACCCTGTTCCTGCAAATGAAACTACCCTCACTATGCTATGG
ACTACTGGGGTCAAGGAACCTCAGTCACCGTCTCCTC
(SEQ ID NO: 13)
EVQLEESGGGLVQPGGSRKLSCAASGFTFSSFGMHWVRQAPEKGLEWVAYISSGSST
LHYADTVKGRFTISRDNPKNTLFLQMKLPSLCYGLLGSRNLSHRLL
HC2
(SEQ ID NO: 14)
GATGTGCAGCTGGTGGAGTCTGGGGGAGGTTTAGTGCAGCCTGGAGGGTCCCGG
AAACTCTCCTGTGCAGCCTCTGGATTCACTTTCAGTAGCTATGGAATTCACTGGG
TTCGTCAGGTTCCAGAGAAGGGGCTGGAGTGGGTCGCATTTATTAGTAGTAGCAA
TTCTACCATCTACTATGCAGACACAGTGAAGGGCCGATTCACCATCTCCAGAGAC
AATGCCGAGAACACCCTGTTCCTGCAAATGACCAGTCTAAGGTCTGAGGACACG
GCCATGTATTACTGTGCAAGACCCCTTTACTACTATGGTAGCGTTATGGACTACT
GGGGTCAAGGAACCTCTGTCACCGTCTCCTCA
(SEQ ID NO: 15)
DVQLVESGGGLVQPGGSRKLSCAASGFTFSSYGIHWVRQVPEKGLEWVAFISSSNSTI
YYADTVKGRFTISRDNAENTLFLQMTSLRSEDTAMYYCARPLYYYGSVMDYWGQG
TSVTVSS
HC3
(SEQ ID NO: 16)
GAGGTCCAGCTGCAACAATCTGGACCTGAGCTGGTGAAGCCTGGGGCTTCAGTG
AAGATATCCTGTAAGGCTTCTGGATACACGTTCACTGACTACTACATGAACTGGA
TGAAGCAGAGCCATGGAAAGAGTCTTGAGTGGATTGGAGATATTAATCCTAACA
ATGGTGGTACTAGCTACAACCAGAAGTTCAAGGGCAAGGCCACATTGACTGTAG
ACAAGTCCTCCAGCACAGCCTACATGGAACTCCGCAGCCTGACATCTGAGGACT
CTGCAGTCTATTACTGTGCAAGACCTTATTACTACGGTAGTAGCTACGACTACTG
GGGCCAAGGCACCACTCTCACAGTCTCCTCA
(SEQ ID NO: 17)
EVQLQQSGPELVKPGASVKISCKASGYTFTDYYMNWMKQSHGKSLEWIGDINPNNG
GTSYNQKFKGKATLTVDKSSSTAYMELRSLTSEDSAVYYCARPYYYGSSYDYWGQ
GTTLIVS
CDRL1
(SEQ ID NO: 18)
QSIVHXNGTY
(SEQ ID NO: 19)
QSIVHRNGNTY
(SEQ ID NO: 20)
QSIVHSNGNTY
(SEQ ID NO: 21)
ENIGSY
CDRL2
(SEQ ID NO: 22)
KVS
(SEQ ID NO: 23)
AAT
CDRL3
(SEQ ID NO: 24)
FQGSXVPXT
(SEQ ID NO: 25)
FQGSYVPPT
(SEQ ID NO: 26)
FQGSHVPLT
(SEQ ID NO: 27)
QHYYSTLVT
LC1
(SEQ ID NO: 28)
GACATTGTGATCACCCAAACTCCACTCTCCCTGCCTGTCAGTCTTGGAGATCAAG
CCTCCATCTCTTGCAGATCTAGTCAGAGCATTGTACATAGGAATGGAAACACCTA
TTTAGAATGGTACTTGCAGCAACCAGGCCAGTCTCCAAAGCTCCTGATCTACAAA
GTTTCCAACCGATTTTCTGGGGTCCCAGACAGGTTCAGTGGCAGTGGATCAGGGA
CAGATTTCACACTCAAGATCAGCAGAGTGGAGGCTGAAGATCTGGGAGTTTATT
ACTGCTTTCAAGGTTCATATGTTCCTCCGACGTTCGGTGGAGGCACCAAGCTGGA
AATCAAA
(SEQ ID NO: 29)
DIVITQTPLSLPVSLGDQASISCRSSQSIVHRNGNTYLEWYLQQPGQSPKLLIYKVSNR
FSGVPDRFSGSGSGTDFTLKISRVEAEDLGVYYCFQGSYVPPTFGGGTKLEIK
LC2
(SEQ ID NO: 30)
GATGTTTTGATGACCCAAACTCCACTCTCCCTGCCTGTCAGTCTTGGAGATCAAG
CCTCCATCTCTTGCAGATCTAGTCAGAGCATTGTACATAGTAATGGAAACACCTA
TTTAGAATGGTACCTGCAGAAACCAGGCCAGTCTCCAAAGCTCCTGATCTACAAA
GTTTCCAACCGATTTTCTGGGGTCCCAGACAGGTTCAGTGGCAGTGGATCAGGGA
CAGATTTCACACTCAAGATAAGTAGAGTGGAGGCTGAGGATCTGGGAGTTTATT
ACTGCTTTCAAGGTTCACATGTTCCTCTCACGTTCGGTGCAGGGACCAAGCTGGA
ACTGAAA
(SEQ ID NO: 31)
DVLMTQTPLSLPVSLGDQASISCRSSQSIVHSNGNTYLEWYLQKPGQSPKLLIYKVSN
RFSGVPDRFSGSGSGTDFTLKISRVEAEDLGVYYCFQGSHVPLTFGAGTKLELK
LC3
(SEQ ID NO: 32)
GACATCCAGATGACTCAGTCTCCAGCTTCCCTGTCTGCATCTGTGGGAGAAACTG
TCACCATCACATGTCGAGCAAGTGAAAATATTGGCAGTTATTTAGCATGGTATCA
GCAGAAACAGGGAAAATCTCCTCAGCTCCTGGTCTATGCTGCAACACTCTTAGCA
GATGGTGTGCCATCAAGGTTCAGTGGCAGTGGATCAGGCACACAGTTTTCTCTCA
AGATCAACAGCCTGCAGTCTGAAGATGTTGCGAGATATTACTGTCAACATTATTA
TAGTACTCTGGTCACGTTCGGTGCTGGGACCAAGCTGGAACTGAAA
(SEQ ID NO: 33)
DIQMTQSPASLSASVGETVTITCRASENIGSYLAWYQQKQGKSPQLLVYAATLLADG
VPSRFSGSGSGTQFSLKINSLQSEDVARYYCQHYYSTLVTFGAGTKLELK
Ig constant regions
Human IgD constant region, Uniprot: P01880
SEQ ID NO: 34
APTKAPDVFPIISGCRHPKDNSPVVLACLITGYHPTSVTVTWYMGTQSQPQRTFPEIQ
RRDSYYMTSSQLSTPLQQWRQGEYKCVVQHTASKSKKEIFRWPESPKAQASSVPTA
QPQAEGSLAKATTAPATTRNTGRGGEEKKKEKEKEEQEERETKTPECPSHTQPLGVY
LLTPAVQDLWLRDKATFTCFVVGSDLKDAHLTWEVAGKVPTGGVEEGLLERHSNG
SQSQHSRLTLPRSLWNAGTSVFCTLNHPSLPPQRLMALREPAAQAPVKLSLNLLASS
DPPEAASWLLCEVSGFSPPNILLMWLEDQREVNTSGFAPARPPPQPGSTTFWAWSVL
RVPAPPSPQPATYTCVVSHEDSRTLLNASRSLEVSYVTDHGPMK
Human IgG1 constant region, Uniprot: P01857
SEQ ID NO: 35
ASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSCDKTHTCPPCPAP
ELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPRE
PQVYTLPPSRDELTKNQVSLTCLVKGFYPSDIAVEWESNGQPENNYKTTPPVLDSDG
SFFLYSKLTVDKSRWQQGNVFSCSVMHEALHNHYTQKSLSLSPGK
Human IgG2 constant region, Uniprot: P01859
SEQ ID NO: 36
ASTKGPSVFPLAPCSRSTSESTAALGCLVKDYFPEPVTVSWNSGALTSGVHTFPAVLQ
SSGLYSLSSVVTVPSSNFGTQTYTCNVDHKPSNTKVDKTVERKCCVECPPCPAPPVA
GPSVFLFPPKPKDTLMISRTPEVTCVVVDVSHEDPEVQFNWYVDGVENHNAKTKPR
EEQFNSTFRVVSVLTVVHQDWLNGKEYKCKVSNKGLPAPIEKTISKTKGQPREPQVY
TLPPSREEMTKNQVSLTCLVKGFYPSDISVEWESNGQPENNYKTTPPMLDSDGSFFL
YSKLTVDKSRWQQGNVFSCSVMHEALHNHYTQKSLSLSPGK
Human IgG3 constant region, Uniprot: P01860
SEQ ID NO: 37
ASTKGPSVFPLAPCSRSTSGGTAALGCLVKDYFPEPVTVSWNSGALTSGVHTFPAVL
QSSGLYSLSSVVTVPSSSLGTQTYTCNVNHKPSNTKVDKRVELKTPLGDTTHTCPRC
PEPKSCDTPPPCPRCPEPKSCDTPPPCPRCPEPKSCDTPPPCPRCPAPELLGGPSVFLFPP
KPKDTLMISRTPEVTCVVVDVSHEDPEVQFKWYVDGVEVHNAKTKPREEQYNSTFR
VVSVLTVLHQDWLNGKEYKCKVSNKALPAPIEKTISKTKGQPREPQVYTLPPSREEM
TKNQVSLTCLVKGFYPSDIAVEWESSGQPENNYNTTPPMLDSDGSFFLYSKLTVDKS
RWQQGNIFSCSVMHEALHNRFTQKSLSLSPGK
Human IgM constant region, Uniprot: P01871
SEQ ID NO: 38
GSASAPTLFPLVSCENSPSDTSSVAVGCLAQDFLPDSITLSWKYKNNSDISSTRGFPSV
LRGGKYAATSQVLLPSKDVMQGTDEHVVCKVQHPNGNKEKNVPLPVIAELPPKVSV
FVPPRDGFFGNPRKSKLICQATGFSPRQIQVSWLREGKQVGSGVTTDQVQAEAKESG
PTTYKVTSTLTIKESDWLGQSMFTCRVDHRGLTFQQNASSMCVPDQDTAIRVFAIPPS
FASIFLTKSTKLTCLVTDLTTYDSVTISWTRQNGEAVKTHTNISESHPNATFSAVGEAS
ICEDDWNSGERFTCTVTHTDLPSPLKQTISRPKGVALHRPDVYLLPPAREQLNLRESA
TITCLVTGFSPADVFVQWMQRGQPLSPEKYVTSAPMPEPQAPGRYFAHSILTVSEEE
WNTGETYTCVAHEALPNRVTERTVDKSTGKPTLYNVSLVMSDTAGTCY
Human IgG4 constant region, Uniprot: P01861
SEQ ID NO: 39
ASTKGPSVFPLAPCSRSTSESTAALGCLVKDYFPEPVTVSWNSGALTSGVHTFPAVLQ
SSGLYSLSSVVTVPSSSLGTKTYTCNVDHKPSNTKVDKRVESKYGPPCPSCPAPEFLG
GPSVFLFPPKPKDTLMISRTPEVTCVVVDVSQEDPEVQFNWYVDGVEVHNAKTKPR
EEQFNSTYRVVSVLTVLHQDWLNGKEYKCKVSNKGLPSSIEKTISKAKGQPREPQVY
TLPPSQEEMTKNQVSLTCLVKGFYPSDIAVEWESNGQPENNYKTTPPVLDSDGSFFL
YSRLTVDKSRWQEGNVFSCSVMHEALHNHYTQKSLSLSLGK
Human IgA1 constant region, Uniprot: P01876
SEQ ID NO: 40
ASPTSPKVFPLSLCSTQPDGNVVIACLVQGFFPQEPLSVTWSESGQGVTARNFPPSQD
ASGDLYTTSSQLTLPATQCLAGKSVTCHVKHYTNPSQDVTVPCPVPSTPPTPSPSTPP
TPSPSCCHPRLSLHRPALEDLLLGSEANLTCTLTGLRDASGVTFTWTPSSGKSAVQGP
PERDLCGCYSVSSVLPGCAEPWNHGKTFTCTAAYPESKTPLTATLSKSGNTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGKPTHVNVSVV
MAEVDGTCY
Human IgA2 constant region, Uniprot: P01877
SEQ ID NO: 41
ASPTSPKVFPLSLDSTPQDGNVVVACLVQGFFPQEPLSVTWSESGQNVTARNFPPSQD
ASGDLYTTSSQLTLPATQCPDGKSVTCHVKHYTNPSQDVTVPCPVPPPPPCCHPRLSL
HRPALEDLLLGSEANLTCTLTGLRDASGATFTWTPSSGKSAVQGPPERDLCGCYSVS
SVLPGCAQPWNHGETFTCTAAHPELKTPLTANITKSGNTFRPEVHLLPPPSEELALNE
LVTLTCLARGFSPKDVLVRWLQGSQELPREKYLTWASRQEPSQGTTTFAVTSILRVA
AEDWKKGDTFSCMVGHEALPLAFTQKTIDRMAGKPTHVNVSVVMAEVDGTCY
Human Ig kappa constant region, Uniprot: P01834
SEQ ID NO: 42
TVAAPSVFIFPPSDEQLKSGTASVVCLLNNFYPREAKVQWKVDNALQSGNSQESVTE
QDSKDSTYSLSSTLTLSKADYEKHKVYACEVTHQGLSSPVTKSFNRGEC
B7-H4
(SEQ ID NO: 43)
IGEDGILSCTFEPDIKLSDIVIQWLKEGVLGLVHEFKEGKDELSEQDEMFRGRTAVFA
DQVIVGNASLRLKNVQLTDAGTYKCYIITSKGKGNANLEYKTGAFSMPEVNVDYNA
SSETLRCEAPRWFPQPTVVWASQVDQGANFSEVSNTSFELNSENVTMKVVSVLYNV
TINNTYSCMIENDIAKATGDIKVTESEIKRRSHLQLLNSKA
(SEQ ID NO: 44)
IGEDGILSCTFEPDIKLSDIVIQWLKEGVLGLVHEFKEGKDELSEQDEMFRGRTAVFA
DQVIVGNASLRLKNVQLTDAGTYKCYIITSKGKGNANLEYKTGAFSMPEVNVDYNA
SSETLRCEAPRWFPQPTVVWASQVDQGANFSEVSNTSFELNSENVTMKVVSVLYNV
TINNTYSCMIENDIAKATGDIKVTESEIKRRSHLQLLNSKADKTHTCPPCPAPELLGGP
SVFLFPPKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLDSDGSFFLYS
KLTVDKSRWQQGNVFSCSVMHEALHNHYTQKSLSLSPGK
CAR Components
Human CD8 alpha hinge domain, SEQ. ID NO: 45:
PAKPTTTPAPRPPTPAPTIASQPLSLRPEACRPAAGGAVHTRGLDFACDIY
Mouse CD8 alpha hinge domain, SEQ. ID NO: 46:
KVNSTTTKPVLRTPSPVHPTGTSQPQRPEDCRPRGSVKGTGLDFACDIY
Cat CD8 alpha hinge domain, SEQ. ID NO: 47:
PVKPTTTPAPRPPTQAPITTSQRVSLRPGTCQPSAGSTVEASGLDLSCDIY
Human CD8 alpha transmembrane domain, SEQ. ID NO: 48:
IYIWAPLAGTCGVLLLSLVIT
Mouse CD8 alpha transmembrane domain, SEQ. ID NO: 49:
IWAPLAGICVALLLSLIITLI
Rat CD8 alpha transmembrane domain, SEQ. ID NO: 50:
IWAPLAGICAVLLLSLVITLI
The 4-1BB costimulatory signaling region, SEQ. ID NO: 51:
KRGRKKLLYIFKQPFMRPVQTTQEEDGCSCRFPEEEEGGCEL
The CD3 zeta signaling domain, SEQ. ID NO: 52:
RVKFSRSADAPAYQQGQNQLYNELNLGRREEYDVLDKRRGRDREMGGKPRRKNPQ
EGLYNELQKDKMAEAYSEIGMKGERRRGKGHDGLYQGLSTATKDTYDALHMQALP
PR

1. An isolated antibody comprising a heavy chain (HC) immunoglobulin variable domain sequence and a light chain (LC) immunoglobulin variable domain sequence, wherein the antibody binds to an epitope of human B7-H4 comprising the amino acid sequence IGEDGILSCTFEPDIKLSDIVIQWLKEGVLGLVHEFKEGKDELSEQDEMFRGRTAVFADQ VIVGNASLRLKNVQLTDAGTYKCYIITSKGKGNANLEYKTGAFSMPEVNVDYNASSETL RCEAPRWFPQPTVVWASQVDQGANF SEVSNTSFELNSENVTMKVVSVLYNVTINNTYS CMIENDIAKATGDIKVTESEIKRRSHLQLLNSKA (SEQ ID NO: 43), or an equivalent thereof.

2. The antibody of claim 1, wherein
(a) the HC comprises a CDRH3 sequence ARPLYYYGSVMDY (SEQ ID NO: 10) or ARPYYYGSSYDY (SEQ ID NO: 11) or an equivalent thereof; or
(b) the LC comprises a CDRL3 sequence FQGSYVPPT (SEQ ID NO: 25), FQGSHVPLT (SEQ ID NO: 26), QHYYSTLVT (SEQ ID NO: 27), or an equivalent of each thereof; or
(c) the HC comprises a CDRH3 sequence ARPLYYYGSVMDY (SEQ ID NO: 10) or ARPYYYGSSYDY (SEQ ID NO: 11), and wherein the LC comprises a CDRL3 sequence FQGSYVPPT (SEQ ID NO: 25), FQGSHVPLT (SEQ ID NO: 26), QHYYSTLVT (SEQ ID NO:
27), or an equivalent of each thereof.

3. The antibody of claim 1, wherein the HC further comprises a CDRH2 sequence ISSGSSTL (SEQ ID NO: 6), ISSSNSTI (SEQ ID NO: 7), or INPNNGGT (SEQ ID NO: 8), or an equivalent of each thereof.

4. The antibody of claim 1, wherein the HC further comprises a CDRH1 sequence GFTFSSFG (SEQ ID NO: 2), GFTFSSYG (SEQ ID NO: 3), or GYTFTDY (SEQ ID NO: 4), or an equivalent of each thereof.

5. The antibody of claim 1, wherein the LC further comprises a CDRL2 sequence KVS (SEQ ID NO: 22) or AAT (SEQ ID NO: 23), or an equivalent of each thereof.

6. The antibody of claim 1, wherein the LC further comprises a CDRL1 sequence QSIVHRNGNTY (SEQ ID NO: 19), QSIVHSNGNTY (SEQ ID NO: 20), or ENIGSY (SEQ ID NO: 21), or an equivalent of each thereof.

7. The antibody of claim 1, wherein the HC comprises
(a) a HC CDRH1 comprising the amino acid sequence GFTFSSFG (SEQ ID NO: 2), GFTFSSYG (SEQ ID NO: 3), or GYTFTDY (SEQ ID NO: 4), or an equivalent of each thereof; and/or
(b) a HC CDRH2 comprising the amino acid sequence ISSGSSTL (SEQ ID NO: 6), ISSSNSTI (SEQ ID NO: 7), or INPNNGGT (SEQ ID NO: 8), or an equivalent of each thereof; and/or
(c) a HC CDRH3 comprising the amino acid sequence ARPLYYYGSVMDY (SEQ ID NO: 10) or ARPYYYGSSYDY (SEQ ID NO: 11), or an equivalent of each thereof; and/or the LC comprises
(a) a LC CDR1 comprising the amino acid sequence QSIVHRNGNTY (SEQ ID NO: 19), QSIVHSNGNTY (SEQ ID NO: 20), or ENIGSY (SEQ ID NO: 21), or an equivalent of each thereof and/or
(b) a LC CDR2 comprising the amino acid sequence KVS (SEQ ID NO: 22) or AAT (SEQ ID NO: 23), or an equivalent of each thereof; and/or
(c) a LC CDR3 comprising the amino acid sequence FQGSYVPPT (SEQ ID NO: 25), FQGSHVPLT (SEQ ID NO: 26), QHYYSTLVT (SEQ ID NO: 27), or an equivalent of each thereof.

8. The antibody of claim 1, wherein the HC comprises
(a) a HC CDRH1 comprising the amino acid sequence GFTFSSFG (SEQ ID NO: 2), GFTFSSYG (SEQ ID NO: 3), or GYTFTDY (SEQ ID NO: 4), or an equivalent of each thereof; and/or
(b) a HC CDRH2 comprising the amino acid sequence ISSGSSTL (SEQ ID NO: 6), ISSSNSTI (SEQ ID NO: 7), or INPNNGGT (SEQ ID NO: 8), or an equivalent of each thereof; and/or
(c) a HC CDRH3 comprising the amino acid sequence ARPLYYYGSVMDY (SEQ ID NO: 10) or ARPYYYGSSYDY (SEQ ID NO: 11), or an equivalent of each thereof; and/or the LC comprises
(a) a LC CDRL1 comprising the amino acid QSIVHRNGNTY (SEQ ID NO: 19), QSIVHSNGNTY (SEQ ID NO: 20), or ENIGSY (SEQ ID NO: 21), or an equivalent of each thereof; and/or
(b) a LC CDRL2 comprising the amino acid sequence KVS (SEQ ID NO: 22) or AAT (SEQ ID NO: 23), or an equivalent of each thereof; and/or
(c) a LC CDRL3 comprising the amino acid sequence FQGSYVPPT (SEQ ID NO: 25), FQGSHVPLT (SEQ ID NO: 26), QHYYSTLVT (SEQ ID NO: 27), or an equivalent of each thereof.

9. The antibody of claim 1, wherein the HC immunoglobulin variable domain sequence comprises the amino acid sequence of SEQ ID NOs:
13, 15, or 17, or an equivalent of each thereof.

10. The antibody of claim 1, wherein the LC immunoglobulin variable domain sequence comprises the amino acid sequence of SEQ ID NOs: 29, 31, or 33, or an equivalent of each thereof.

11. The antibody of claim 1, wherein the HC immunoglobulin variable domain sequence comprises the amino acid sequence of SEQ ID NOs: 13, 15, or 17, or an equivalent of each thereof, and wherein the LC immunoglobulin variable domain sequence comprises the amino acid sequence of SEQ ID NOs: 29, 31, or 33, or an equivalent of each thereof.

12. The antibody of claim 1, wherein the antibody is selected from the group of: a monoclonal antibody, a chimeric antibody or a humanized antibody.

13. An antigen binding fragment of the antibody of claim 1, wherein the antigen binding fragment is selected from the group consisting of Fab, F(ab′)2, Fab′, scFv, and Fv.

14. The antibody or antigen binding fragment of claim 1, wherein wherein an equivalent comprises an polypeptide having at least 80% amino acid identity to polypeptide or a polypeptide that is encoded by a polynucleotide that hybridizes under conditions of high stringency to the complement of a polynucleotide encoding the polypeptide, wherein conditions of high stringency comprise incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC.

15. An isolated ex vivo complex comprising an antibody of claim 1 or an antigen binding fragment thereof, or an equivalent of each thereof, and optionally a detectable label.

16. An isolated ex vivo cell comprising the complex of claim 15.

17. A method of detecting B7-H4 in a biological sample comprising contacting the sample with the antibody of claim 1 or an antigen binding fragment thereof, or an equivalent of each thereof, and detecting a complex formed by the binding of the antibody or antigen binding fragment to B7-H4.

18. The method of claim 17, wherein the sample comprises a cell sample or a tissue sample.

19. The method of claim 17, wherein the sample is obtained from a subject that is diagnosed as having, suspected as having, or at risk of having cancer.

20. The method of claim 19, wherein the cancer is selected from a solid tumor cancer, optionally, breast, colon, chorio-carcinoma, prostate or ovarian cancer.

21. The method of claim 17, wherein the detection comprises one or more of immunohistochemistry (IHC), Western blotting, Flow cytometry or ELISA.

22. A method of detecting a pathological cell in a sample isolated from a subject, comprising
(a) detecting the level of B7-H4 in a biological sample from the subject by detecting a complex formed by the antibody or antigen binding fragment of claim 1 binding to B7-H4 in the sample; and
(b) comparing the levels of B7-H4 observed in step (a) with the levels of B7-H4 observed in a control biological sample;
wherein the pathological cell is detected when the level of B7-H4 is elevated compared to that observed in the control biological sample and the pathological cell is not detected when the level of B7-H4 is not elevated as compared to the observed in the control biological sample.

23. The method of claim 22, wherein the biological sample of the subject comprises one or more of a sample isolated from a solid tumor, optionally, breast, colon, chorio-carcinoma, prostate or ovarian cancer.

24. The method of claim 22, wherein the detection comprises one or more of immunohistochemistry (IHC), Western Blotting, Flow cytometry or ELISA.

25. The method of claim 22, further comprising isolating the biological sample from the subject.

26. The method of claim 25, wherein the subject is a mammal.

27. The method of claim 26, wherein the mammal is selected from the group of: a murine, feline, canine, ovine, bovine, simian, and a human.

28. A B7-H4-specific antibody or antigen binding fragment thereof, wherein the antibody or antigen binding fragment has the same epitope specificity as the antibody of claim 1.

29. A kit for detecting B7-H4 comprising an antibody of claim 1 or an antigen binding fragment thereof, or an equivalent of each thereof, and instructions for use.

30. A method of detecting B7-H4 in a tumor sample comprising:
(a) contacting the sample with an antibody or an antigen binding fragment of the antibody, wherein the antibody comprises a heavy chain (HC) immunoglobulin variable domain sequence and a light chain (LC) immunoglobulin variable domain sequence, wherein the antibody binds to an epitope of human B7-H4 comprising the amino acid sequence,
wherein the HC comprises
(i) a HC CDRH1 comprising the amino acid sequence GFTFSSFG (SEQ ID NO: 2), GFTFSSYG (SEQ ID NO: 3), or GYTFTDY (SEQ ID NO: 4), or an equivalent of each thereof;
(ii) a HC CDRH2 comprising the amino acid sequence ISSGSSTL (SEQ ID NO: 6), ISSSNSTI (SEQ ID NO: 7), or INPNNGGT (SEQ ID NO: 8), or an equivalent of each thereof; and
(iii) a HC CDRH3 comprising the amino acid sequence ARPLYYYGSVMDY (SEQ ID NO: 10) or ARPYYYGSSYDY (SEQ ID NO: 11), or an equivalent of each thereof; and
the LC comprises
(i) a LC CDRL1 comprising the amino acid QSIVHRNGNTY (SEQ ID NO: 19), QSIVHSNGNTY (SEQ ID NO: 20), or ENIGSY (SEQ ID NO: 21), or an equivalent of each thereof; and
(ii) a LC CDRL2 comprising the amino acid sequence KVS (SEQ ID NO: 22) or AAT (SEQ ID NO: 23), or an equivalent of each thereof; and
(iii) a LC CDRL3 comprising the amino acid sequence FQGSYVPPT (SEQ ID NO: 25), FQGSHVPLT (SEQ ID NO: 26), QHYYSTLVT (SEQ ID NO: 27), or an equivalent of each thereof.
(b) detecting a complex formed by the binding of the antibody or antigen binding fragment to B7-H4.

31. A chimeric antigen receptor (CAR) comprising: (a) an antigen binding domain of an anti-B7-H4 antibody; (b) a CD8 α hinge domain; (c) a CD8 α transmembrane domain; (d) a CD28 costimulatory signaling region and/or a 4-1BB costimulatory signaling region; and (e) a CD3 zeta signaling domain.

32. The CAR of claim 31, comprises an anti-B7-H4 heavy chain variable region and an anti-B7-H4 light chain variable region that comprises the antigen binding domain of the anti-B7-H4 antibody.

33. The CAR of claim 32, further comprising a linker polypeptide located between the anti-B7-H4 heavy chain variable region and the anti-B7-H4 light chain variable region.

34. The CAR of claim 32, wherein the anti-B7-H4 heavy chain variable region comprises a CDR region comprising any one of SEQ ID NOs: 1 to 11 or an equivalent thereof.

35. The CAR of claim 32, wherein the anti-B7-H4 heavy chain variable region comprises any one of SEQ ID NOs: 12 to 17 or an equivalent thereof.

36. The CAR of claim 32, wherein the anti-B7-H4 light chain variable region a CDR region comprising any one of SEQ ID NOs: 18 to 27 or an equivalent thereof.

37. The CAR of claim 32, wherein the anti-B7-H4 light chain variable region a CDR region comprising any one of SEQ ID NOs: 28 to 33 or an equivalent thereof.

38. The CAR of claim 32, wherein the anti-B7-H4 heavy chain variable region and light chain variable regions are joined by a glycine-serine linker.

39. The CAR of claim 31, further comprising a detectable marker or a purification marker.

40. The CAR of claim 34, wherein an equivalent comprises an polypeptide having at least 80% amino acid identity to polypeptide or a polypeptide that is encoded by a polynucleotide that hybridizes under conditions of high stringency to the complement of a polynucleotide encoding the polypeptide, wherein conditions of high stringency comprise incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC.

41. An isolated nucleic acid sequence encoding the CAR of claim 31 or its complement or an equivalent of each thereof.

42. The isolated nucleic acid of claim 41, further comprising a Kozak consensus sequence located upstream of the antigen binding domain of the anti-B7-H4 antibody or B7-H4 ligand.

43. The isolated nucleic sequence of claim 41, further comprising an antibiotic resistance polynucleotide.

44. The isolated nucleic acid of claim 41, wherein an equivalent thereof comprises an polynucleotide having at least 80% nucleic acid identity to the nucleic acid or one that hybridizes under conditions of high stringency to the complement of the nucleic acid, or the nucleic acid, wherein conditions of high stringency comprise incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC.

45. A vector comprising the isolated nucleic acid sequence of claim 41.

46. The vector of claim 45, wherein the vector is a plasmid.

47. The vector of claim 45, wherein the vector is a lentiviral vector.

48. An isolated cell comprising the CAR of claim 31.

49. The isolated cell of claim 48, wherein the cell is a T-cell.

50. The isolated cell of claim 48, wherein the cell is an NK-cell.

51. An isolated nucleic acid encoding the isolated antibody of claim 1 or its complement.

52. A composition comprising a carrier and one or more of: an isolated cell comprising the CAR of claim 31.

53. A method of producing B7-H4 CAR expressing cells comprising:
(i) transducing a population of isolated cells with a nucleic acid sequence encoding the CAR of claim 31; and
(ii) selecting a subpopulation of said isolated cells that have been successfully transduced with said nucleic acid sequence of step (i) thereby producing B7-H4 CAR expressing cells.

54. The method of claim 53, wherein the isolated cells are selected from a group consisting of T-cells and NK-cells.

55. A method of inhibiting the growth of a tumor in a subject in need thereof, comprising administering to the subject an effective amount of the isolated cell of claim 48.

56. The method of claim 55, wherein the isolated cells are autologous to the subject being treated.

57. The method of claim 55 or 56, wherein the tumor is a solid tumor, optionally, breast, colon or a chorio-carcinoma.

58. The method of claim 55, wherein the tumor cells express or overexpress B7-H4.

59. A method of treating a cancer patient in need thereof, comprising administering to the subject an effective amount of the isolated cell of claim 48.

60. The method of claim 59, wherein the isolated cells are autologous to the subject being treated.

61. The method of claim 59 or 60, wherein the tumor is a solid tumor, optionally breast, colon or a chorio-carcinoma.

62. The method of claim 59, wherein the cancer cells express or overexpress B7-H4.

63. The method of claim 59, wherein the subject is a human patient.

64. A method for determining if a patient is likely to respond or is not likely to B7-H4 CAR therapy, comprising contacting a tumor sample isolated from the patient with an effective amount of an anti-B7-H4 antibody and detecting the presence of any antibody bound to the tumor sample, wherein the presence of antibody bound to the tumor sample indicates that the patient is likely to respond to the B7-H4 CAR therapy and the absence of antibody bound to the tumor sample indicates that the patient is not likely to respond to the B7-H4 therapy.

65. The method of claim 64, further comprising administering an effective amount of the B7-H4 CAR therapy to the patient that is determined likely to respond to the B7-H4 CAR therapy.


SEQUENCE LISTING (67 sequences)

SEQ ID NO: 1 - protein, 4 aa, Artificial Sequence
  Description of Artificial Sequence Synthetic peptide
  MOD_RES (2)..(2): Any amino acid
GXTF

SEQ ID NO: 2 - protein, 8 aa, Artificial Sequence
  Description of Artificial Sequence Synthetic peptide
GFTFSSFG

SEQ ID NO: 3 - protein, 8 aa, Artificial Sequence
  Description of Artificial Sequence Synthetic peptide
GFTFSSYG

SEQ ID NO: 4 - protein, 7 aa, Artificial Sequence
  Description of Artificial Sequence Synthetic peptide
GYTFTDY

SEQ ID NO: 5 - protein, 7 aa, Artificial Sequence
  Description of Artificial Sequence Synthetic peptide
  MOD_RES (4)..(6): Any amino acid
ISSXXXT

SEQ ID NO: 6 - protein, 8 aa, Artificial Sequence
  Description of Artificial Sequence Synthetic peptide
ISSGSSTL

SEQ ID NO: 7 - protein, 8 aa, Artificial Sequence
  Description of Artificial Sequence Synthetic peptide
ISSSNSTI

SEQ ID NO: 8 - protein, 8 aa, Artificial Sequence
  Description of Artificial Sequence Synthetic peptide
INPNNGGT

SEQ ID NO: 9 - protein, 6 aa, Artificial Sequence
  Description of Artificial Sequence Synthetic peptide
  MOD_RES (4)..(4): Any amino acid
ARPXYY

SEQ ID NO: 10 - protein, 13 aa, Artificial Sequence
  Description of Artificial Sequence Synthetic peptide
ARPLYYYGSVMDY

SEQ ID NO: 11 - protein, 12 aa, Artificial Sequence
  Description of Artificial Sequence Synthetic peptide
ARPYYYGSSYDY

SEQ ID NO: 12 - DNA, 309 bp, Artificial Sequence
  Description of Artificial Sequence Synthetic polynucleotide
gaggtgcagctggaggagtctgggggaggcttagtgcagcctggagggtcccggaaactctcctgtgcagcctctggattcactttcagtagctttggaatgcactgggttcgtcaggctccagagaaggggctggagtgggtcgcatacattagtagtggcagtagtaccctccactatgcagacacagtgaagggccgattcaccatctccagagacaatcccaagaacaccctgttcctgcaaatgaaactaccctcactatgctatggactactggggtcaaggaacctcagtcaccgtctcctc

SEQ ID NO: 13 - protein, 103 aa, Artificial Sequence
  Description of Artificial Sequence Synthetic polypeptide
EVQLEESGGGLVQPGGSRKLSCAASGFTFSSFGMHWVRQAPEKGLEWVAYISSGSSTLHYADTVKGRFTISRDNPKNTLFLQMKLPSLCYGLLGSRNLSHRLL

SEQ ID NO: 14 - DNA, 360 bp, Artificial Sequence
  Description of Artificial Sequence Synthetic polynucleotide
gatgtgcagctggtggagtctgggggaggtttagtgcagcctggagggtcccggaaactctcctgtgcagcctctggattcactttcagtagctatggaattcactgggttcgtcaggttccagagaaggggctggagtgggtcgcatttattagtagtagcaattctaccatctactatgcagacacagtgaagggccgattcaccatctccagagacaatgccgagaacaccctgttcctgcaaatgaccagtctaaggtctgaggacacggccatgtattactgtgcaagacccctttactactatggtagcgttatggactactggggtcaaggaacctctgtcaccgtctcctca

SEQ ID NO: 15 - protein, 120 aa, Artificial Sequence
  Description of Artificial Sequence Synthetic polypeptide
DVQLVESGGGLVQPGGSRKLSCAASGFTFSSYGIHWVRQVPEKGLEWVAFISSSNSTIYYADTVKGRFTISRDNAENTLFLQMTSLRSEDTAMYYCARPLYYYGSVMDYWGQGTSVTVSS

SEQ ID NO: 16 - DNA, 357 bp, Artificial Sequence
  Description of Artificial Sequence Synthetic polynucleotide
gaggtccagctgcaacaatctggacctgagctggtgaagcctggggcttcagtgaagatatcctgtaaggcttctggatacacgttcactgactactacatgaactggatgaagcagagccatggaaagagtcttgagtggattggagatattaatcctaacaatggtggtactagctacaaccagaagttcaagggcaaggccacattgactgtagacaagtcctccagcacagcctacatggaactccgcagcctgacatctgaggactctgcagtctattactgtgcaagaccttattactacggtagtagctacgactactggggccaaggcaccactctcacagtctcctca

SEQ ID NO: 17 - protein, 118 aa, Artificial Sequence
  Description of Artificial Sequence Synthetic polypeptide
EVQLQQSGPELVKPGASVKISCKASGYTFTDYYMNWMKQSHGKSLEWIGDINPNNGGTSYNQKFKGKATLTVDKSSSTAYMELRSLTSEDSAVYYCARPYYYGSSYDYWGQGTTLTVS

SEQ ID NO: 18 - protein, 10 aa, Artificial Sequence
  Description of Artificial Sequence Synthetic peptide
  MOD_RES (6)..(6): Any amino acid
QSIVHXNGTY

SEQ ID NO: 19 - protein, 11 aa, Artificial Sequence
  Description of Artificial Sequence Synthetic peptide
QSIVHRNGNTY

SEQ ID NO: 20 - protein, 11 aa, Artificial Sequence
  Description of Artificial Sequence Synthetic peptide
QSIVHSNGNTY

SEQ ID NO: 21 - protein, 6 aa, Artificial Sequence
  Description of Artificial Sequence Synthetic peptide
ENIGSY

SEQ ID NO: 22 - protein, 3 aa, Artificial Sequence
  Description of Artificial Sequence Synthetic peptide
KVS

SEQ ID NO: 23 - protein, 3 aa, Artificial Sequence
  Description of Artificial Sequence Synthetic peptide
AAT

SEQ ID NO: 24 - protein, 9 aa, Artificial Sequence
  Description of Artificial Sequence Synthetic peptide
  MOD_RES (5)..(5): Any amino acid
  MOD_RES (8)..(8): Any amino acid
FQGSXVPXT

SEQ ID NO: 25 - protein, 9 aa, Artificial Sequence
  Description of Artificial Sequence Synthetic peptide
FQGSYVPPT

SEQ ID NO: 26 - protein, 9 aa, Artificial Sequence
  Description of Artificial Sequence Synthetic peptide
FQGSHVPLT

SEQ ID NO: 27 - protein, 9 aa, Artificial Sequence
  Description of Artificial Sequence Synthetic peptide
QHYYSTLVT

SEQ ID NO: 28 - DNA, 336 bp, Artificial Sequence
  Description of Artificial Sequence Synthetic polynucleotide
gacattgtgatcacccaaactccactctccctgcctgtcagtcttggagatcaagcctccatctcttgcagatctagtcagagcattgtacataggaatggaaacacctatttagaatggtacttgcagcaaccaggccagtctccaaagctcctgatctacaaagtttccaaccgattttctggggtcccagacaggttcagtggcagtggatcagggacagatttcacactcaagatcagcagagtggaggctgaagatctgggagtttattactgctttcaaggttcatatgttcctccgacgttcggtggaggcaccaagctggaaatcaaa

SEQ ID NO: 29 - protein, 112 aa, Artificial Sequence
  Description of Artificial Sequence Synthetic polypeptide
DIVITQTPLSLPVSLGDQASISCRSSQSIVHRNGNTYLEWYLQQPGQSPKLLIYKVSNRFSGVPDRFSGSGSGTDFTLKISRVEAEDLGVYYCFQGSYVPPTFGGGTKLEIK

SEQ ID NO: 30 - DNA, 336 bp, Artificial Sequence
  Description of Artificial Sequence Synthetic polynucleotide
gatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagataagtagagtggaggctgaggatctgggagtttattactgctttcaaggttcacatgttcctctcacgttcggtgcagggaccaagctggaactgaaa

SEQ ID NO: 31 - protein, 112 aa, Artificial Sequence
  Description of Artificial Sequence Synthetic polypeptide
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPLTFGAGTKLELK

SEQ ID NO: 32 - DNA, 321 bp, Artificial Sequence
  Description of Artificial Sequence Synthetic polynucleotide
gacatccagatgactcagtctccagcttccctgtctgcatctgtgggagaaactgtcaccatcacatgtcgagcaagtgaaaatattggcagttatttagcatggtatcagcagaaacagggaaaatctcctcagctcctggtctatgctgcaacactcttagcagatggtgtgccatcaaggttcagtggcagtggatcaggcacacagttttctctcaagatcaacagcctgcagtctgaagatgttgcgagatattactgtcaacattattatagtactctggtcacgttcggtgctgggaccaagctggaactgaaa

SEQ ID NO: 33 - protein, 107 aa, Artificial Sequence
  Description of Artificial Sequence Synthetic polypeptide
DIQMTQSPASLSASVGETVTITCRASENIGSYLAWYQQKQGKSPQLLVYAATLLADGVPSRFSGSGSGTQFSLKINSLQSEDVARYYCQHYYSTLVTFGAGTKLELK

SEQ ID NO: 34 - protein, 384 aa, Homo sapiens
APTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDHGPMK

SEQ ID NO: 35 - protein, 330 aa, Homo sapiens [sequence omitted]

SEQ ID NO: 36 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 37 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 38 - protein, 452 aa, Homo sapiens
GSASAPTLFPLVSCENSPSDTSSVAVGCLAQDFLPDSITLSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVAHEALPNRVTERTVDKSTGKPTLYNVSLVMSDTAGTCY

SEQ ID NO: 39 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 40 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 41 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRMAGKPTHVNVSVVMAEVDGTCY

SEQ ID NO: 42 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 211 aa, Homo sapiens
IGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKA

SEQ ID NO: 44 - protein, 438 aa, Artificial Sequence
  Description of Artificial Sequence Synthetic polypeptide
IGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 51 aa, Homo sapiens
PAKPTTTPAPRPPTPAPTIASQPLSLRPEACRPAAGGAVHTRGLDFACDIY

SEQ ID NO: 46 - protein, 49 aa, Mus musculus
KVNSTTTKPVLRTPSPVHPTGTSQPQRPEDCRPRGSVKGTGLDFACDIY

SEQ ID NO: 47 - protein, 51 aa, Felis catus
PVKPTTTPAPRPPTQAPITTSQRVSLRPGTCQPSAGSTVEASGLDLSCDIY

SEQ ID NO: 48 - protein, 21 aa, Homo sapiens
IYIWAPLAGTCGVLLLSLVIT

SEQ ID NO: 49 - protein, 21 aa, Mus musculus
IWAPLAGICVALLLSLIITLI

SEQ ID NO: 50 - protein, 21 aa, Rattus norvegicus
IWAPLAGICAVLLLSLVITLI

SEQ ID NO: 51 - protein, 42 aa, Unknown
  Description of Unknown 4-1BB costimulatory signaling region
KRGRKKLLYIFKQPFMRPVQTTQEEDGCSCRFPEEEEGGCEL

SEQ ID NO: 52 - protein, 112 aa, Unknown
  Description of Unknown CD3 zeta signaling domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 53 - DNA, 48 bp, Unknown
  Description of Unknown IgG1 heavy chain hinge sequence
ctcgagcccaaatcttgtgacaaaactcacacatgcccaccgtgcccg

SEQ ID NO: 54 - DNA, 81 bp, Unknown
  Description of Unknown CD28 transmembrane region
ttttgggtgctggtggtggttggtggagtcctggcttgctatagcttgctagtaacagtggcctttattattttctgggtg

SEQ ID NO: 55 - DNA, 126 bp, Unknown
  Description of Unknown 4-1BB co-stimulatory signaling region
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 56 - DNA, 123 bp, Unknown
  Description of Unknown CD28 co-stimulatory signaling region
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 57 - DNA, 339 bp, Unknown
  Description of Unknown CD3 zeta signaling region
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 58 - protein, 220 aa, Unknown
  Description of Unknown CD28 polypeptide
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 59 - DNA, 105 bp, Unknown
  Description of Unknown ICOS costimulatory signaling region
acaaaaaagaagtattcatccagtgtgcacgaccctaacggtgaatacatgttcatgagagcagtgaacacagccaaaaaatccagactcacagatgtgacccta

SEQ ID NO: 60 - DNA, 108 bp, Unknown
  Description of Unknown OX40 costimulatory signaling region
agggaccagaggctgccccccgatgcccacaagccccctgggggaggcagtttccggacccccatccaagaggagcaggccgacgcccactccaccctggccaagatc

SEQ ID NO: 61 - protein, 15 aa, Artificial Sequence
  Description of Artificial Sequence Synthetic peptide
GGGGSGGGGSGGGGS

SEQ ID NO: 62 - protein, 5 aa, Artificial Sequence
  Description of Artificial Sequence Synthetic peptide
GGGGS

SEQ ID NO: 63 - DNA, 42 bp, Artificial Sequence
  Description of Artificial Sequence Synthetic primer
tcgatcaagcttgccgccaccatggcttccctggggcagatc

SEQ ID NO: 64 - DNA, 30 bp, Artificial Sequence
  Description of Artificial Sequence Synthetic primer
tgtgtgagttttgtcagcctttgacagctg

SEQ ID NO: 65 - DNA, 36 bp, Artificial Sequence
  Description of Artificial Sequence Synthetic primer
ctaaactcaaaggctgacaaaactcacacatgccca

SEQ ID NO: 66 - DNA, 42 bp, Artificial Sequence
  Description of Artificial Sequence Synthetic primer
tgattaatgatcaatgaattctcatttacccggagacaggga

SEQ ID NO: 67 - protein, 11 aa, Artificial Sequence
  Description of Artificial Sequence Synthetic peptide
RPYYYGSSYDY